SEQUENCE LISTING (22 sequences)

SEQ ID NO: 1 - DNA, 1493 bp, Streptomyces zinciresistens
  source 1..1493: mol_type = other DNA
cctaggatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaa

SEQ ID NO: 2 - DNA, 1012 bp, Suberites domuncula
  source 1..1012: mol_type = other DNA
ggcgcgccaccatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataagtttaaac

SEQ ID NO: 3 - DNA, 816 bp, Cylindrotheca fulsiformis
  source 1..816: mol_type = other DNA
ctcgaggccaccatgaagctgaccgccatctttccgcttcttttcactgctgtcgggtactgtgctgcccaaagcattgctgacttggcagcagccaatctcagcactgaggattccaagtcagctcagctcatctcagcagactcctcggatgacgcatctgattcctctgtcgagtctgtcgacgccgcctcctctgacgtctctggttcctctgtcgaatctgtcgacgtctctggttcctctctggaatccgttgacgtctctggttcctctctggagtccgtcgacgactccagtgaggactccgaagaggaagaacttcgtatcttgtcttccaagaaatccggatcctactactcctacggcaccaagaagtcgggatcctactcgggatactccacaaagaaatccgcatcgcgtcgcatcttgtcctccaagaaatcgggatcctactcgggatattccacaaagaaatccggatcgcgtcgcatcttgtcctccaagaaatcgggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtccgggggtctcagaggctccatgtaaggtacc

SEQ ID NO: 4 - DNA, 1059 bp, Saccharomyces cerevisiae
  source 1..1059: mol_type = other DNA
ggtaccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtcaggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataactcgag

SEQ ID NO: 5 - DNA, 1215 bp, Micrococcus sp. HL-2003
  source 1..1215: mol_type = other DNA
atggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaa

SEQ ID NO: 6 - DNA, 3414 bp, Saccharomyces cerevisiae
  source 1..3414: mol_type = other DNA
aagcttgccaccatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtactgttccaatacaacttttgttttgtttgaaagagcaaaatcagaaaaaaattaactcacatagatgggcatttgaaggctttgcagaattactgcgtcccaatatcgttacattgttagatgctggtactatgccaggtaaagattctatttaccagttatggagagagttcaggaatccaaatgttggtggcgcatgtggtgaaataagaactgatttgggtaagagatttgtaaagttgttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctagtctttaggattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaggctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaaggtacc

SEQ ID NO: 7 - DNA, 1712 bp, Saccharomyces cerevisiae
  source 1..1712: mol_type = other DNA
gaattcaagcttatgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttagctaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaactcgaggatcc

SEQ ID NO: 8 - DNA, 939 bp, Saccharomyces cerevisiae
  source 1..939: mol_type = other DNA
atgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaa

SEQ ID NO: 9 - DNA, 2328 bp, Pyropia yezoensis
  source 1..2328: mol_type = other DNA
ggatccggtaccatggagactgttcgtccaggttgttttcgtgctgttgtttctgttccactgggtcgtctgacttactctttcctggtggacggtcagccgaagtacaacccggactgtccgactctggttaacgaagctggcgtgcgtgtgaacgtacgccacggtggttctgacggtgattgcgacggtgaagctgacgaagaatggggtaaaggtcgtccaagccgtgctcgtcgcgtgctgagccaggtttctggcctggacctgtactctcagcactccaccagcgacatcgtttctatgctggtgttccgtttcttctacctggctatgattccagctggtgcctactacttcttctggctgagcgttcgtggtggtaatcgccaggctccagtttgctgggtggtgttcgttatgagcgaaatcctgtctttcgttagcgcgctgatttccctgtttggcatgtggaagccgatcaaacgtcgttggcgctctctggacgctctgcgtccagctctgccagttgcagattggccaactgtagacgtgatcatttgccactacaaagaggacaccgaacagctgcgtcagactattcgtgcagcgatgaaactggattacccagcccatctgctgcacattctgattgccgacgatggcttcttcccaacctccaagatggtagagcgttctgatattggtctggctctgtaccagacctgtgttgaggaagcgggttatgatccgctgctggaagaaaccatgaatgaacgtggtctggttgaacactatactgttaaggcaggtgacgacctgccgcgtcatgattgcgcggttgaagctcaccagttcgagtttggtccgtatggtgcggacatgtacggtccaggtgccctgccacgtctgtccctggttgctcgtgtgaagccggctgacgctcacaacaaggcaggcaacatcaacaacgtactgtccaactctaacgcagaaggtaagattgttctgttcctggatgctgacatgaagccggtagaatcttacctgctgcgtgttctgccactgatgctggaagagcagcgttccgatagcctgcagagccaactgatccaggcagaggacccagagctgggtgctggtacttctaagagctggcaaatcaaccgtgacattggcttcgttggctgcccacaacgcttcgcgaacgtatccggtgaccatccggattactgtgctcaccgtaacgcgatctactacgacggtatctgcactggtcgtgatggtttcggtatgaccgacttcgttggcactaacgcgtgttggcgtcgcgaagttctgaacgagatcggtggcttcgtttatggtagcgtgactgaagacaccctgactagcaacgaagttcatcgccgtggctacattagccgctacgctgacgaagacctgtgctggggtgaagcaccagttaccgttgcagcagctctgctgcagcgtcaacgttgggctaaaggtgccatcatgaacggtatgcgtatcttcaaaggtgctgctaaagaacgcaaagaaatgctgctgagccgtgagaaaccgtctgaactgtctgagttctacgcatatcgtcgtcagcagcgtaaaccaaacaacactttcgtttctaccatgttctggctggattctactctgtacccactgctgggttggggtgctttcggttacgtgttctgtgctatcttccacctgatcaccgctcaggcaccgatctctccgacctctactcagagcctggctggtgcgtttgtgacctactatctgattcgttacggtgcattcttctctgccttctatgaagtgaacatgaccgacgttctgcgctctcagcagtgttggttctcttactccttcgcccacactgtgggtgtatgggatgctctgtttggtggtgccaagtttggctgggttgctaacactggccaacgtcatcgccgtagctggctggaatggttcaacatcctgactctgggtgctctgctgtccggcattgtgtggcgtctgttcgcgttcattgttatcgaagaagcgtgttctccgtacgagaacttcggtgctgttgcattcggcggttacgtagcgtggatgatggctccagttgctctggttagcctgaacgaacgcctgtcttccgctgacgaatccgagcgtgaaggtaaaccgatgccagtgccgactccgatcatcgctgctgccctgactatcctgggtgttgtgttcctgtctggttgggcaaacgctcgttgtggtatcgaagctcgcggttaaaagcttctcgag

SEQ ID NO: 10 - DNA, 1367 bp, Oryza japonica
  source 1..1367: mol_type = other DNA
ctcgagaagcttttagctggtagtggagatcttagctttcacatccaggttctctttatcatcgatcggtttggtagtctggttgcgaacacgcttgatcttcggagactccaggcctttgtgagcgaaaccgtacagacgcagaacctggttgtcagcaaagttgaacgcacgctccatagaacgcagacaacgttcaaccggatagtcgccgtaagaaccgcacggtttgcagcccacgaagtgagtcacaaatggccagcgttcgtcacccagacctgggtggtggttttccatcatttcttcgtacttatctaccagacctgcccagaagccgtgcaggtagtagctgttttcgataaacactttgttcatccacttctccttctggctcagcagcaggtagatcagggcagactggtcatccgcctcgaacgctggacgacccttcaggttcgcggtcagaatcttgcctgcttcatcacggataaaacctttcggacccatcggggcccacgcgtccagcagatccagggaccactggcagttgcggaacaggaaggaaccggtgttcagagcaatccaggaatgcttttcaaacagcagatcctggtaaccgtgaataatcaggttgcgatcctggtaacgagacagcggcagttcgaaagccatgtcagtgaacagtgcgtcgctgtccatccaccagatccattcaacttccgggtgagacagcatcaggcgacgcagcagtggcagtttcgcccagtagcctgccagttcggtgtccaggtgtgccaggttatgtacgatctcgataccatgcagacgacagtaatcaattttgttcttagtagttttcagcagatagtgatcacccagtgggttgtcgcatggacctggctgggagccagtcaccagcaggatacgcggtttgccaccagccacagtggacggaaagcctggattctgacgcagccaacgacgacgctgaccatcccaatcagaaatctttggacccagtgcatagcgttccactgcagatgcgtacgctgcagcagcctcagtagcgttcagcgcggtcgcgttgaagctgcctgccagatcgcctgcagccgctgcatcgtcgtcggcaccaccgtcagagcggatctcacgcagaatgcgatcgatatcttcaaccgcctttgcacctgccagagcgtctgcatcggttggctggtttggcaggctcaggttcagaccaatggtgccacgcagtaccagaatagtaaccagaccacacagaacagtgatcttaacgttgttgaaggtgcgatgaatcttacgagaacgatgatggtgaccgtgggagctcagcggagatgcac

SEQ ID NO: 11 - DNA, 1365 bp, Medicago trunculata
  source 1..1365: mol_type = other DNA
ctcgagaagcttttacggctgcggatagtcaaacggcagagaagaaacaccgttgtcacccagatccttatgaacgaagccgaatttgcgcataacctggttatctgcaaagttcagggctttcttcatgccgttccagcaagcgtccgcagtgtacatttcgttatacttgccagaacacggctgacagccagtgaagtgagtcacgaatggacgacgccagctgcctttaccatagcctgcatctttcaggtattcttcacggataacaccatacgcttcggatactttctcagcgtagcgacgacgcagtttcggttccaccttctcgatgtcctcgtatttcttgctgatgttggagaaagtttccacgatctcttcccagtaaccttcgaagtagtattcgccttccaggtagattttatcaccccacttctctttctcgatggcgatcaggtatgccagaccggtttgatcatcagattccgggaagaacttgtctttgaaagtgctacgcagggtcttaccccattcttcgtagtctggagactgtggacccatgccagcccatgcctccatgaagtccaggctccattgacagttacgaatcaggaacacacctgcgttcagaccggtccagctgcgtttctcgtggatcaggtgtggccagccgtgtaccaccaggttatggtccttgtaacgtttcagtggcagcttgaaatccatgtcagtgaacagcgcatcgctatccacccaccaaatccattcagcttctgggtgagccatcatagcggctttcactaccggatactttgcccaatacgcaaacattttcggatgcagcagtgcattgttgtagaagatgtcgtagccgtggatgcgggagtaatctactttgttcttgaagaagcgcagcagcagatggtcaccaatcggattcttacacggagacggttgggagccagtcaccatcagcacgcgttcttttgcacctgcagagaaagacgggtggtgtttcagccattcttcacgcttctcatcccagtcttccagtttcacggacatggtatattccatttccggatcgtcgtagaaggtctggtccggtgggtcgaaacgcaggtccggagtagagtaagatacaacattgttcttatggctcttcagagtcaggttgtcgttcgggatcggattagtgaaagaggagaagccccaaaccagcagcagtgcgctgaatgcaccacccaggaacaggaaaccatcagacagaaagatgctggaagatttgttacgcgggatcggtttcgccatgatgctgctgaagtgagaatgagacagttcggtagtaaccatggtaccggatcc

SEQ ID NO: 12 - DNA, 1332 bp, Cyamopsis tetragonoloba
  source 1..1332: mol_type = other DNA
ggatccggtaccatggcgaagtttggtagccgtaataagtctccgaagtggatctctaacggctgctgcttcctgctgggtgcctttactgctctgctgctgctgtggggtctgtgctcctttattatcccgattccgaacaccgatccgaagctgaactctgtagcgacctctctgcgttctctgaacttcccgaagaacccagcagctaccctgccaccgaacctgcagcatgatccaccggatactactttctacgacgatccggaaacctcttatactatggataaaccgatgaagaactgggacgagaaacgtaaggaatggctgctgcatcacccgtccttcggcgcagctgctcgtgacaagatcctgctggtaaccggttcccagccgaaacgctgtcacaacccgattggtgatcacctgctgctgcgtttctttaagaacaaagtggattactgccgtctgcataattacgatatcatctataacaacgcactgctgcacccgaaaatgaactcctattgggctaagtacccagtgatccgtgctgcaatgatggctcatccggaagttgaatgggtgtggtgggttgactctgacgctgtattcactgacatggagtttaaactgccactgaaacgctataagaaccacaatctggtggtgcatggttgggaaggtctggtacgtctgaaccactcctggactggtctgaacgcaggtgtattcctgatccgtaattgccaatggagcctggagttcatggatgtatgggttagcatgggtccacaaactccagaatacgagaaatggggtgagcgtctgcgcgaaaccttcaaagataaagttctgccagactctgatgaccagactgcactggcatacctgatcgcaaccgataacaaagacacctggcgtgagaaaatcttcctggaaagcgagtactacttcgaaggctattggctggaaatcgtaaagacttacgagaacatctccgaacgttacgatgaagttgaacgcaaagtagaaggtctgcgtcgtcgccacgctgagaaggttagcgagaaatacggtgcaatgcgtgaagaatacctgaaagataacaaacgtcgtccgttcatcacccatttcactggttgccagccgtgcaacggtcaccataacccagcatacaatgcgaacgactgttggaatggtatggaacgtgcactgaactttgcagataaccagatcctgcgtacctacggttaccatcgtcagaatctgctggacaagtccgtttctccactgccgttcggttatccagcagcttaaaagcttctcgag

SEQ ID NO: 13 - DNA, 20 bp, synthetic construct
  misc_feature 1..20: note = Synthetic construct
  source 1..20: mol_type = other DNA
taatacgactcactataggg

SEQ ID NO: 14 - DNA, 1386 bp, synthetic construct
  misc_feature 1..1386: note = Synthetic construct
  source 1..1386: mol_type = other DNA
ctcgaggaattctcactggaacagcgcgtcactcgacaggccattcttctccagaatctcccgcaggcgcttcagcgcctcgacctggatctgacgaacccgctcgcgggtcaggccgatttcctggccgacctcttccagcgtgctgctttcgtgaccgcgcaagccgaagcggcgaatcaccacctcacgctgcttgtcggtgagttccgtcagctgctttcgctgagatcgtcatcctgcagcagctcgcacggatcggtggggcgatcgtcggtgagcgtatccagcagggtcttgtccgagtccggaccaagagagacgtctaccgaagtcacccgttcgttcaggccgagcatgcgcttgacctcggcgaccggcttctccagcaggttggcgatttcttcgggtgaaggttcgtggtcgagcttgtgggtcagttcccgcgccgcacgcaggtagacgttgagctccttgaccacatggatcggcaagcgaatggtccgggtctggttcatgatggcccgctcgatggtctggcggatccaccaggtggcgtaggtcgagaaccggaatccgcgctccggatcgaacttctccacggcgcggatcaggcctaggttgccttcctcgatcaggtcgagcagggacagtccgcgattgacatagcgccgggcgatcttcaccaccaaccgcaggttgctctcgatcatccgcttccgaccagcgggatcgcccttctgcgccagacgagcgaagtggacttcctcttcgggcgtcaacaggggcgagaaaccgatttcgttgagatacagctgcgttgcgtccaacgcgcgcgtgtagtcgatgtgcttgtgttgtttggaagagaaggaagtggtggcttttggagttgcccggggagaaggctgctcgtcggcagacgactcgtccagcatgatgccgggctccaggaggagcacttcatcatcgtggtcaaactccggcccttcttttttgagtgccatgtcgttatcccttgcatgagttcgactcaagcccgggcgattcctttcccgctggacacgcccggacccgctcacctacatgatgtgggcgggcgaactcccggtcagcgacgtggcaaatattgcagtggatcgacaggcttaccctggcggcgaatctcgaagtgcagcttcacccgatcggttcctgtggagcccatctcggcaatcgattgccctaccttgacctgttgcccttcccgcaccagcagcctgcggttgtgaccgtaggcactcacgtaggtctcgttgtgtttgatgatgaccaactcgccgtagccccgcaaaccactaccggcgtatacaacggtcccaccagacgcagccaggtaccaagctt

SEQ ID NO: 15 - DNA, 890 bp, synthetic construct
  misc_feature 1..890: note = Synthetic construct
  source 1..890: mol_type = other DNA
ggattcaaagaggagaaatactagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaagctt

SEQ ID NO: 16 - DNA, 13637 bp, synthetic construct
  misc_feature 1..13637: note = Synthetic construct
  source 1..13637: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgaggccaccatgaagctgaccgccatctttccgcttcttttcactgctgtcgggtactgtgctgcccaaagcattgctgacttggcagcagccaatctcagcactgaggattccaagtcagctcagctcatctcagcagactcctcggatgacgcatctgattcctctgtcgagtctgtcgacgccgcctcctctgacgtctctggttcctctgtcgaatctgtcgacgtctctggttcctctctggaatccgttgacgtctctggttcctctctggagtccgtcgacgactccagtgaggactccgaagaggaagaacttcgtatcttgtcttccaagaaatccggatcctactactcctacggcaccaagaagtcgggatcctactcgggatactccacaaagaaatccgcatcgcgtcgcatcttgtcctccaagaaatcgggatcctactcgggatattccacaaagaaatccggatcgcgtcgcatcttgtcctccaagaaatcgggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtccgggggtctcagaggctccatgtaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 17 - DNA, 15564 bp, synthetic construct
  misc_feature 1..15564: note = Synthetic construct
  source 1..15564: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggccaccatggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaacccgggtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagccgcggccaccatgacagatcgttgtccaggtagggatgctccacacttagcagtcattggagcaggtccagctggcttagcagcagcattagctgctgctgctagaggtgttcgtgtaaccttgttggatgctgaaccagaagcaggaggccaattctatagacagccagcagcagctttacgtgctagaaggccacaagcattacaccatcagtggcgtacctttgccagattgagacacggattagccaggcacattgcagcaggtagagttagacatgctagagaacaccatgtttggtttgctgagagagctcctgatggtggattcaccgttcatgctttgactggtccaggtagaggagatccagcagaagtgagagcagatgcagtcttgttggcaactggtggtcacgagactgtgttgccattcccaggttggaccttgccaggtgttgtcacagctggaggtgcccaagccatgttgaaggcaggtttagttacatctggcaacaccgcagtcgtagctggtactggtccattgttgttgccagtagctacaggtttagctgctgctggtgttgacgtaagagcattagtcgaaagtgctgatcctggtgccttaccaagacaggcacgtgctttggcagctcaacctggcaagttggctgaaggtgctttgtatgctggtcaattgttgaggcacagagtgcgtgtcttgactagacacactgtcgttgaagcacatggtacagagaggttggaagcagttactgttgcagccttggatgcaggtggacgtactagacctggcactgctagaagaatagcatgtgcaactttagctgtgggtcatggtatgttgccacatacagacttggcagacgccttaggctgccgtttagcaggtccagcagttcatgcagatgatgaacaaagaactgatgttcctggtgtgtgggcagcaggagagtgtactggcgtaggtggtgcagctttgtctttggctgagggtcatatcgctggcagaagtgcagcagccagattgttaggagcacctccaggtcccgacgcatggccagaggcagctagaacaagagcaaggttgagagctttctccgctgtattggatgctgtttacactcctcctcctggttggggtgagagagtcaccgacgcaaccgttgtatgcaggtgtgaagaagttacagcaggtgcaatccgtgcttctgtgagggaattgggagctggtgacgtacgtactgtaaagttgttgactagagctggcatgggatggtgtcagggaagaatgtgtgctcctgctgtcgctggattggcaggttgtgctttcactcctagtcgtagaccattcgctaggccagtgcctttgggagtgttggccagagctggtgaagatgcaggtggcgatggaggcagagctgaggatcaaggtgaaggagatggacgtgctgctggagcaggaggttgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgcttgtcacagtggtagtactgggtctactggggtttgcttctgcagcccagcccaagtttgaatttgtagaagaatggcagctgtggaagtccactcactctaagatgtacgagtcacagttaatggaactcgaaagacatctgacgtggctctccaataagaaatatatcgagcaacacaatgtcaactcacacattttcggttttactctggcaatgaaccagtttggagatctgagtgaattggagtatgctaactatcttggccagtatcgcattgaggataaaaaatctggcaactactcaaagacttttcagcgtgatcctctacaggactaccctgaagctgtagactggagaaccaaaggagctgtcacggctgtcaaggaccagggagactgtggtgctagctatgctttcagtgctatgggtgctttggagggtgctaatgctttagccaagggaaatgcagtatctctcagtgaacagaacatcattgattgctcgattccttacggtaaccacggttgtcatggaggcaatatgtatgatgcttttttgtatgtcatcgctaacgagggggtcgatcaggacagtgcatatccatttgtaggaaagcaatccagctgcaactataatagtaaatacaaaggtacatcaatgtcggggatggtgtcaatcaaaagtggtagtgagtctgacttacaagcagctgtttcaaacgttggccctgtatctgttgctattgatggtgctaacagtgccttcaggttttactacagtggtgtctatgactcatcacgatgctctagtagtagtcttaaccacgcaatggtagtcactggatacggatcatacaatgggaaaaaatactggctggccaagaatagctggggaactaactggggtaacagtggctatgtgatgatggctcgcaacaagtacaaccagtgtggaattgctaccgatgcatcttatcccaccctataagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgaggccaccatgaagctgaccgccatctttccgcttcttttcactgctgtcgggtactgtgctgcccaaagcattgctgacttggcagcagccaatctcagcactgaggattccaagtcagctcagctcatctcagcagactcctcggatgacgcatctgattcctctgtcgagtctgtcgacgccgcctcctctgacgtctctggttcctctgtcgaatctgtcgacgtctctggttcctctctggaatccgttgacgtctctggttcctctctggagtccgtcgacgactccagtgaggactccgaagaggaagaacttcgtatcttgtcttccaagaaatccggatcctactactcctacggcaccaagaagtcgggatcctactcgggatactccacaaagaaatccgcatcgcgtcgcatcttgtcctccaagaaatcgggatcctactcgggatattccacaaagaaatccggatcgcgtcgcatcttgtcctccaagaaatcgggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtcctccaaaaaatccggatcctactcgggatccaagggctccaagcgtcgcaacttgtcctccaagaaatccggatcctactcgggatccaagggttccaagcgtcgcatcttgtccgggggtctcagaggctccatgtaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 18 - DNA, 14634 bp, synthetic construct
  misc_feature 1..14634: note = Synthetic construct
  source 1..14634: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtactgttccaatacaacttttgttttgtttgaaagagcaaaatcagaaaaaaattaactcacatagatgggcatttgaaggctttgcagaattactgcgtcccaatatcgttacattgttagatgctggtactatgccaggtaaagattctatttaccagttatggagagagttcaggaatccaaatgttggtggcgcatgtggtgaaataagaactgatttgggtaagagatttgtaaagttgttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctagtctttaggattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaggctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttagctaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgagatgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 19 - DNA, 16558 bp, synthetic construct
  misc_feature 1..16558: note = Synthetic construct
  source 1..16558: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaacccgggtaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcgcgccaccatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtactgttccaatacaacttttgttttgtttgaaagagcaaaatcagaaaaaaattaactcacatagatgggcatttgaaggctttgcagaattactgcgtcccaatatcgttacattgttagatgctggtactatgccaggtaaagattctatttaccagttatggagagagttcaggaatccaaatgttggtggcgcatgtggtgaaataagaactgatttgggtaagagatttgtaaagttgttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctagtctttaggattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaggctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtcactcctttacatcattcttctattcacacaattcttactactgccaaccgatgcctttgataggtctgctaacacaaatattgctgtttattggggtcaaaactcagcaggaacgcaagaatccttagctacttactgtgaatcttctgatgctgatattttcctattatctttcttgaaccaatttccaacccttggtttgaactttgccaacgcatgctctgatactttttctgatggcttacttcactgcacccagattgctgaagatattgaaacttgccagtccctaggaaagaaagttctattatcattaggtggtgcatctggtagctacctcttttcagatgattctcaagcggaaacttttgcacaaactttatgggatactttcggtgaaggtacaggtgccagtgagagaccatttgactcagcagtcgttgatggttttgattttgatattgaaaacaacaacgaagtaggctatagtgcgttagctaccaagttaagaactttgtttgccgaaggtacaaagcaatattacctttctgccgcaccacaatgtccatacccggatgcttctgttggtgacttgttggaaaatgcagacattgattttgcgttcatccaattttacaataattactgcagtgtgagtggtcaattcaattgggatacttggttaacctatgctcaaactgtatccccaaataaaaatatcaaactgttcttaggtttacctggttctgcttctgctgctggctctggttatatttctgacacttctttattggaatcaactattgcagatattgcctcttcaagttcttttggtggtattgcgttatgggatgcatctcaagccttttccaacgagctaaatggtgaaccatatgttgagattttgaagaatttgctaacaagtgctagccagaccgccactactacagttgccacctcaaaaacctcagcagcctcaacttcatctgcttcaacttcatctgcttcaacttctcagaaaaagaccacacaatctacgacatctacacaaagtaaaagcaaagttactttatctccaactgcaagcagcgctatcaaaacatcaattactcaaactacaaaaacattgacgagtagcaccaagacaaaatctagtctaggtaccaccacaacagagagcactttaaattcagttgctatcacaagtatgaaaactactctatcttcccaaataaccagtgctgccttggtgacccctcaaacaactactactagcatagtttcttcggccccaattcaaacagctatcactagtactctttcgccagcaacgaagagttcttctgtcgtttccctacagacagctactactagtacgctttccccaacaacgaccagtacaagctcaggtagtacaagctcaggtagtacaagctcagacagtacagctcgtacattggctaaagaattgaatgctcaatatgcggctggtaaattgaacggtaaatctacctgtactgaaggtgaaattgcatgctctgctgatgggaagttcgccgtttgtgatcatagcgcttgggtttacatggaatgtgcttctggaaccacatgttatgcttatgactccggcgactcagtctatacccaatgtaatttctcttatttggaaagcaattacttttaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgagatgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 20 - DNA, 11513 bp, synthetic construct
  misc_feature 1..11513: note = Synthetic construct
  source 1..11513: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttaccatggagactgttcgtccaggttgttttcgtgctgttgtttctgttccactgggtcgtctgacttactctttcctggtggacggtcagccgaagtacaacccggactgtccgactctggttaacgaagctggcgtgcgtgtgaacgtacgccacggtggttctgacggtgattgcgacggtgaagctgacgaagaatggggtaaaggtcgtccaagccgtgctcgtcgcgtgctgagccaggtttctggcctggacctgtactctcagcactccaccagcgacatcgtttctatgctggtgttccgtttcttctacctggctatgattccagctggtgcctactacttcttctggctgagcgttcgtggtggtaatcgccaggctccagtttgctgggtggtgttcgttatgagcgaaatcctgtctttcgttagcgcgctgatttccctgtttggcatgtggaagccgatcaaacgtcgttggcgctctctggacgctctgcgtccagctctgccagttgcagattggccaactgtagacgtgatcatttgccactacaaagaggacaccgaacagctgcgtcagactattcgtgcagcgatgaaactggattacccagcccatctgctgcacattctgattgccgacgatggcttcttcccaacctccaagatggtagagcgttctgatattggtctggctctgtaccagacctgtgttgaggaagcgggttatgatccgctgctggaagaaaccatgaatgaacgtggtctggttgaacactatactgttaaggcaggtgacgacctgccgcgtcatgattgcgcggttgaagctcaccagttcgagtttggtccgtatggtgcggacatgtacggtccaggtgccctgccacgtctgtccctggttgctcgtgtgaagccggctgacgctcacaacaaggcaggcaacatcaacaacgtactgtccaactctaacgcagaaggtaagattgttctgttcctggatgctgacatgaagccggtagaatcttacctgctgcgtgttctgccactgatgctggaagagcagcgttccgatagcctgcagagccaactgatccaggcagaggacccagagctgggtgctggtacttctaagagctggcaaatcaaccgtgacattggcttcgttggctgcccacaacgcttcgcgaacgtatccggtgaccatccggattactgtgctcaccgtaacgcgatctactacgacggtatctgcactggtcgtgatggtttcggtatgaccgacttcgttggcactaacgcgtgttggcgtcgcgaagttctgaacgagatcggtggcttcgtttatggtagcgtgactgaagacaccctgactagcaacgaagttcatcgccgtggctacattagccgctacgctgacgaagacctgtgctggggtgaagcaccagttaccgttgcagcagctctgctgcagcgtcaacgttgggctaaaggtgccatcatgaacggtatgcgtatcttcaaaggtgctgctaaagaacgcaaagaaatgctgctgagccgtgagaaaccgtctgaactgtctgagttctacgcatatcgtcgtcagcagcgtaaaccaaacaacactttcgtttctaccatgttctggctggattctactctgtacccactgctgggttggggtgctttcggttacgtgttctgtgctatcttccacctgatcaccgctcaggcaccgatctctccgacctctactcagagcctggctggtgcgtttgtgacctactatctgattcgttacggtgcattcttctctgccttctatgaagtgaacatgaccgacgttctgcgctctcagcagtgttggttctcttactccttcgcccacactgtgggtgtatgggatgctctgtttggtggtgccaagtttggctgggttgctaacactggccaacgtcatcgccgtagctggctggaatggttcaacatcctgactctgggtgctctgctgtccggcattgtgtggcgtctgttcgcgttcattgttatcgaagaagcgtgttctccgtacgagaacttcggtgctgttgcattcggcggttacgtagcgtggatgatggctccagttgctctggttagcctgaacgaacgcctgtcttccgctgacgaatccgagcgtgaaggtaaaccgatgccagtgccgactccgatcatcgctgctgccctgactatcctgggtgttgtgttcctgtctggttgggcaaacgctcgttgtggtatcgaagctcgcggttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatggcgaagtttggtagccgtaataagtctccgaagtggatctctaacggctgctgcttcctgctgggtgcctttactgctctgctgctgctgtggggtctgtgctcctttattatcccgattccgaacaccgatccgaagctgaactctgtagcgacctctctgcgttctctgaacttcccgaagaacccagcagctaccctgccaccgaacctgcagcatgatccaccggatactactttctacgacgatccggaaacctcttatactatggataaaccgatgaagaactgggacgagaaacgtaaggaatggctgctgcatcacccgtccttcggcgcagctgctcgtgacaagatcctgctggtaaccggttcccagccgaaacgctgtcacaacccgattggtgatcacctgctgctgcgtttctttaagaacaaagtggattactgccgtctgcataattacgatatcatctataacaacgcactgctgcacccgaaaatgaactcctattgggctaagtacccagtgatccgtgctgcaatgatggctcatccggaagttgaatgggtgtggtgggttgactctgacgctgtattcactgacatggagtttaaactgccactgaaacgctataagaaccacaatctggtggtgcatggttgggaaggtctggtacgtctgaaccactcctggactggtctgaacgcaggtgtattcctgatccgtaattgccaatggagcctggagttcatggatgtatgggttagcatgggtccacaaactccagaatacgagaaatggggtgagcgtctgcgcgaaaccttcaaagataaagttctgccagactctgatgaccagactgcactggcatacctgatcgcaaccgataacaaagacacctggcgtgagaaaatcttcctggaaagcgagtactacttcgaaggctattggctggaaatcgtaaagacttacgagaacatctccgaacgttacgatgaagttgaacgcaaagtagaaggtctgcgtcgtcgccacgctgagaaggttagcgagaaatacggtgcaatgcgtgaagaatacctgaaagataacaaacgtcgtccgttcatcacccatttcactggttgccagccgtgcaacggtcaccataacccagcatacaatgcgaacgactgttggaatggtatggaacgtgcactgaactttgcagataaccagatcctgcgtacctacggttaccatcgtcagaatctgctggacaagtccgtttctccactgccgttcggttatccagcagcttaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 21 - DNA, 13440 bp, synthetic construct
  misc_feature 1..13440: note = Synthetic construct
  source 1..13440: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaacccgggtaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcgcgccaccatggagactgttcgtccaggttgttttcgtgctgttgtttctgttccactgggtcgtctgacttactctttcctggtggacggtcagccgaagtacaacccggactgtccgactctggttaacgaagctggcgtgcgtgtgaacgtacgccacggtggttctgacggtgattgcgacggtgaagctgacgaagaatggggtaaaggtcgtccaagccgtgctcgtcgcgtgctgagccaggtttctggcctggacctgtactctcagcactccaccagcgacatcgtttctatgctggtgttccgtttcttctacctggctatgattccagctggtgcctactacttcttctggctgagcgttcgtggtggtaatcgccaggctccagtttgctgggtggtgttcgttatgagcgaaatcctgtctttcgttagcgcgctgatttccctgtttggcatgtggaagccgatcaaacgtcgttggcgctctctggacgctctgcgtccagctctgccagttgcagattggccaactgtagacgtgatcatttgccactacaaagaggacaccgaacagctgcgtcagactattcgtgcagcgatgaaactggattacccagcccatctgctgcacattctgattgccgacgatggcttcttcccaacctccaagatggtagagcgttctgatattggtctggctctgtaccagacctgtgttgaggaagcgggttatgatccgctgctggaagaaaccatgaatgaacgtggtctggttgaacactatactgttaaggcaggtgacgacctgccgcgtcatgattgcgcggttgaagctcaccagttcgagtttggtccgtatggtgcggacatgtacggtccaggtgccctgccacgtctgtccctggttgctcgtgtgaagccggctgacgctcacaacaaggcaggcaacatcaacaacgtactgtccaactctaacgcagaaggtaagattgttctgttcctggatgctgacatgaagccggtagaatcttacctgctgcgtgttctgccactgatgctggaagagcagcgttccgatagcctgcagagccaactgatccaggcagaggacccagagctgggtgctggtacttctaagagctggcaaatcaaccgtgacattggcttcgttggctgcccacaacgcttcgcgaacgtatccggtgaccatccggattactgtgctcaccgtaacgcgatctactacgacggtatctgcactggtcgtgatggtttcggtatgaccgacttcgttggcactaacgcgtgttggcgtcgcgaagttctgaacgagatcggtggcttcgtttatggtagcgtgactgaagacaccctgactagcaacgaagttcatcgccgtggctacattagccgctacgctgacgaagacctgtgctggggtgaagcaccagttaccgttgcagcagctctgctgcagcgtcaacgttgggctaaaggtgccatcatgaacggtatgcgtatcttcaaaggtgctgctaaagaacgcaaagaaatgctgctgagccgtgagaaaccgtctgaactgtctgagttctacgcatatcgtcgtcagcagcgtaaaccaaacaacactttcgtttctaccatgttctggctggattctactctgtacccactgctgggttggggtgctttcggttacgtgttctgtgctatcttccacctgatcaccgctcaggcaccgatctctccgacctctactcagagcctggctggtgcgtttgtgacctactatctgattcgttacggtgcattcttctctgccttctatgaagtgaacatgaccgacgttctgcgctctcagcagtgttggttctcttactccttcgcccacactgtgggtgtatgggatgctctgtttggtggtgccaagtttggctgggttgctaacactggccaacgtcatcgccgtagctggctggaatggttcaacatcctgactctgggtgctctgctgtccggcattgtgtggcgtctgttcgcgttcattgttatcgaagaagcgtgttctccgtacgagaacttcggtgctgttgcattcggcggttacgtagcgtggatgatggctccagttgctctggttagcctgaacgaacgcctgtcttccgctgacgaatccgagcgtgaaggtaaaccgatgccagtgccgactccgatcatcgctgctgccctgactatcctgggtgttgtgttcctgtctggttgggcaaacgctcgttgtggtatcgaagctcgcggttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatggcgaagtttggtagccgtaataagtctccgaagtggatctctaacggctgctgcttcctgctgggtgcctttactgctctgctgctgctgtggggtctgtgctcctttattatcccgattccgaacaccgatccgaagctgaactctgtagcgacctctctgcgttctctgaacttcccgaagaacccagcagctaccctgccaccgaacctgcagcatgatccaccggatactactttctacgacgatccggaaacctcttatactatggataaaccgatgaagaactgggacgagaaacgtaaggaatggctgctgcatcacccgtccttcggcgcagctgctcgtgacaagatcctgctggtaaccggttcccagccgaaacgctgtcacaacccgattggtgatcacctgctgctgcgtttctttaagaacaaagtggattactgccgtctgcataattacgatatcatctataacaacgcactgctgcacccgaaaatgaactcctattgggctaagtacccagtgatccgtgctgcaatgatggctcatccggaagttgaatgggtgtggtgggttgactctgacgctgtattcactgacatggagtttaaactgccactgaaacgctataagaaccacaatctggtggtgcatggttgggaaggtctggtacgtctgaaccactcctggactggtctgaacgcaggtgtattcctgatccgtaattgccaatggagcctggagttcatggatgtatgggttagcatgggtccacaaactccagaatacgagaaatggggtgagcgtctgcgcgaaaccttcaaagataaagttctgccagactctgatgaccagactgcactggcatacctgatcgcaaccgataacaaagacacctggcgtgagaaaatcttcctggaaagcgagtactacttcgaaggctattggctggaaatcgtaaagacttacgagaacatctccgaacgttacgatgaagttgaacgcaaagtagaaggtctgcgtcgtcgccacgctgagaaggttagcgagaaatacggtgcaatgcgtgaagaatacctgaaagataacaaacgtcgtccgttcatcacccatttcactggttgccagccgtgcaacggtcaccataacccagcatacaatgcgaacgactgttggaatggtatggaacgtgcactgaactttgcagataaccagatcctgcgtacctacggttaccatcgtcagaatctgctggacaagtccgtttctccactgccgttcggttatccagcagcttaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 22 - DNA, 10123 bp, synthetic construct
  misc_feature 1..10123: note = Synthetic construct
  source 1..10123: mol_type = other DNA
ggggaattgtgagcggataacaattcccctctagaaataattttgtttaactttaagaaggagatataccatggtgtttatcgtcaatcttttctcctgcaccttatctgaaaccacggttagctcaataaaatctgaagctacggttagctcaacatttactgccgtcacggccctgcaattggtggctgagggtaagttgcagtcggcgaagggtttcggtggtggtacgattcactacccaaccctcgcggccgaagcaccctggtggacgccgggccaaggccatggttacgaggcgatcacctacggctggctggtcggcgaactgctgcgccgcgccgatgggcgtgggcctggtctgttaggcgctattgccgtggttcctggttacgtttcttacgagaactctatcaagtggtggggaccgcgtctggcttcttggggctttgtcgttgcacggccgttgggcctggactttcatgtgggcctggcggatgaagagttttatcgtgttgcccatatagcgcgcagcaaagccaatgcagcactagataacattgctgatgacaccgtcggcagtatagatcctaagcggttgggcgctattggctggtcaggtggcggcggcgcgcttaaactggcaacggagcgcagcacagtacgagccattttgaccagtactaataaacctgaatggcgacgcttcgataaattcttatgtgcctgcgaggatgaccggattgctgagactaagaaatatgccaacgcgttttataaaaatgccgacatgctcgaagagttgacccgtgaacacagtatcgggccggataaaacattattgacacaaactcggtttggcttggggtgcttggatcaaccgcaagcaggggttaaaattcattttgaagagtaccttgatcaaacccatggatttatcaatttgacgccagtttcacataaggcgagagcaaatctgattcagatgcctaatgccacattcggccttggcccgcgtgcttttgggcatcctggtgcaggtggatcggtaggttttgccgaccccgaacacgatgtagcgtttggtttcgtgactaatacattggggccttatgtagttgagtttaaaagccgtcatccctcattttatgcatataaagatggattggtgctgactggaaatgacgtcgactatgtgactgattactatgcaacaaagcatgctgtacatttagatgatccacgtgcacagaagttggtcggaatattggccggttgtctgtaaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggagactgttcgtccaggttgttttcgtgctgttgtttctgttccactgggtcgtctgacttactctttcctggtggacggtcagccgaagtacaacccggactgtccgactctggttaacgaagctggcgtgcgtgtgaacgtacgccacggtggttctgacggtgattgcgacggtgaagctgacgaagaatggggtaaaggtcgtccaagccgtgctcgtcgcgtgctgagccaggtttctggcctggacctgtactctcagcactccaccagcgacatcgtttctatgctggtgttccgtttcttctacctggctatgattccagctggtgcctactacttcttctggctgagcgttcgtggtggtaatcgccaggctccagtttgctgggtggtgttcgttatgagcgaaatcctgtctttcgttagcgcgctgatttccctgtttggcatgtggaagccgatcaaacgtcgttggcgctctctggacgctctgcgtccagctctgccagttgcagattggccaactgtagacgtgatcatttgccactacaaagaggacaccgaacagctgcgtcagactattcgtgcagcgatgaaactggattacccagcccatctgctgcacattctgattgccgacgatggcttcttcccaacctccaagatggtagagcgttctgatattggtctggctctgtaccagacctgtgttgaggaagcgggttatgatccgctgctggaagaaaccatgaatgaacgtggtctggttgaacactatactgttaaggcaggtgacgacctgccgcgtcatgattgcgcggttgaagctcaccagttcgagtttggtccgtatggtgcggacatgtacggtccaggtgccctgccacgtctgtccctggttgctcgtgtgaagccggctgacgctcacaacaaggcaggcaacatcaacaacgtactgtccaactctaacgcagaaggtaagattgttctgttcctggatgctgacatgaagccggtagaatcttacctgctgcgtgttctgccactgatgctggaagagcagcgttccgatagcctgcagagccaactgatccaggcagaggacccagagctgggtgctggtacttctaagagctggcaaatcaaccgtgacattggcttcgttggctgcccacaacgcttcgcgaacgtatccggtgaccatccggattactgtgctcaccgtaacgcgatctactacgacggtatctgcactggtcgtgatggtttcggtatgaccgacttcgttggcactaacgcgtgttggcgtcgcgaagttctgaacgagatcggtggcttcgtttatggtagcgtgactgaagacaccctgactagcaacgaagttcatcgccgtggctacattagccgctacgctgacgaagacctgtgctggggtgaagcaccagttaccgttgcagcagctctgctgcagcgtcaacgttgggctaaaggtgccatcatgaacggtatgcgtatcttcaaaggtgctgctaaagaacgcaaagaaatgctgctgagccgtgagaaaccgtctgaactgtctgagttctacgcatatcgtcgtcagcagcgtaaaccaaacaacactttcgtttctaccatgttctggctggattctactctgtacccactgctgggttggggtgctttcggttacgtgttctgtgctatcttccacctgatcaccgctcaggcaccgatctctccgacctctactcagagcctggctggtgcgtttgtgacctactatctgattcgttacggtgcattcttctctgccttctatgaagtgaacatgaccgacgttctgcgctctcagcagtgttggttctcttactccttcgcccacactgtgggtgtatgggatgctctgtttggtggtgccaagtttggctgggttgctaacactggccaacgtcatcgccgtagctggctggaatggttcaacatcctgactctgggtgctctgctgtccggcattgtgtggcgtctgttcgcgttcattgttatcgaagaagcgtgttctccgtacgagaacttcggtgctgttgcattcggcggttacgtagcgtggatgatggctccagttgctctggttagcctgaacgaacgcctgtcttccgctgacgaatccgagcgtgaaggtaaaccgatgccagtgccgactccgatcatcgctgctgccctgactatcctgggtgttgtgttcctgtctggttgggcaaacgctcgttgtggtatcgaagctcgcggttaaggtaccagaaataattttgtttaactttaagaaggagactcgagatggcgaagtttggtagccgtaataagtctccgaagtggatctctaacggctgctgcttcctgctgggtgcctttactgctctgctgctgctgtggggtctgtgctcctttattatcccgattccgaacaccgatccgaagctgaactctgtagcgacctctctgcgttctctgaacttcccgaagaacccagcagctaccctgccaccgaacctgcagcatgatccaccggatactactttctacgacgatccggaaacctcttatactatggataaaccgatgaagaactgggacgagaaacgtaaggaatggctgctgcatcacccgtccttcggcgcagctgctcgtgacaagatcctgctggtaaccggttcccagccgaaacgctgtcacaacccgattggtgatcacctgctgctgcgtttctttaagaacaaagtggattactgccgtctgcataattacgatatcatctataacaacgcactgctgcacccgaaaatgaactcctattgggctaagtacccagtgatccgtgctgcaatgatggctcatccggaagttgaatgggtgtggtgggttgactctgacgctgtattcactgacatggagtttaaactgccactgaaacgctataagaaccacaatctggtggtgcatggttgggaaggtctggtacgtctgaaccactcctggactggtctgaacgcaggtgtattcctgatccgtaattgccaatggagcctggagttcatggatgtatgggttagcatgggtccacaaactccagaatacgagaaatggggtgagcgtctgcgcgaaaccttcaaagataaagttctgccagactctgatgaccagactgcactggcatacctgatcgcaaccgataacaaagacacctggcgtgagaaaatcttcctggaaagcgagtactacttcgaaggctattggctggaaatcgtaaagacttacgagaacatctccgaacgttacgatgaagttgaacgcaaagtagaaggtctgcgtcgtcgccacgctgagaaggttagcgagaaatacggtgcaatgcgtgaagaatacctgaaagataacaaacgtcgtccgttcatcacccatttcactggttgccagccgtgcaacggtcaccataacccagcatacaatgcgaacgactgttggaatggtatggaacgtgcactgaactttgcagataaccagatcctgcgtacctacggttaccatcgtcagaatctgctggacaagtccgtttctccactgccgttcggttatccagcagcttaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

(19) United States
(12) Patent Application Publication
(10) Pub. No.: US 2026/0009039 A1
(43) Pub. Date: Jan. 8, 2026

(54) ANTI-MICROBIAL AND UV-PROTECTIVE EXTRACTS AND METHODS OF MAKING AND USING THEREOF

(71) Applicant: BIOCAPITAL HOLDINGS, LLC, Houston, TX (US)

(72) Inventors: Raul CUERO RENGIFO, CYPRESS, TX (US); Juliana LONDONO MURILLO, Manizales (CO)

(21) Appl. No.: 19/312,553

(22) Filed: Aug. 28, 2025

Related U.S. Application Data
(63) Continuation of application No. 18/317,548, filed on May 15, 2023, now Pat. No. 12,460,212.
Application No. 18/317,548 is a continuation of application No. 16/971,347, filed on Aug. 20, 2020, now Pat. No. 11,692,192.
(60) Provisional application No. 62/634,251, filed on Feb. 23, 2018.
(60) Provisional application No. 62/754,291, filed on Nov. 1, 2018.

(51) Int. Cl. C12N 15/52; A01N 63/50; A61K 8/9728; A61K 8/99; C05F 11/00; C07K 14/405; C08G 18/32; C08G 18/76; C08G 101/00; C09D 5/16; C09D 5/32; C12N 9/02; C12N 9/04; C12N 9/20; C12N 9/64; C12N 15/62; C12N 15/70; C12N 15/81

(52) U.S. Cl. CPC C12N 15/52; A01N 63/50; A61K 8/9728; A61K 8/99; C05F 11/00; C08G 18/3218; C08G 18/7671; C09D 5/1637; C09D 5/32; C12N 9/0006; C12N 9/0091; C12N 9/20; C12N 15/62; C12N 15/81; C12Y 101/01001; C07K 14/405; C07K 2319/00; C08G 2101/00; C12N 9/641; C12N 15/70

(57) ABSTRACT

Described herein are anti-microbial and UV-protective biological devices and extracts produced therefrom. The biological devices include microbial cells transformed with a DNA construct containing genes for producing proteins such as, for example, zinc-related protein/oxidase, silicatein, silaffin, and alcohol dehydrogenase. In some instances, the biological devices also include a gene for lipase. Methods for producing and using the devices are also described herein. Finally, compositions and methods for using the devices and extracts to kill microbial species or prevent microbial growth and to reduce or prevent UV-induced damage or exposure to materials, items, plants, and human and animal subjects are described herein. Also disclosed are biological devices producing polyactive carbohydrates and carbo sugars, as well as compositions and articles incorporating both extracts from these devices and the anti-microbial and UV-protective extracts.

### CROSS REFERENCE TO RELATED APPLICATIONS

This application is a continuation of U.S. nonprovisional application Ser. No. 18/317,548 filed May 15, 2023, which is a continuation of U.S. nonprovisional application Ser. No. 16/971,347, filed Aug. 20, 2020, which is a US national phase application of international application no. PCT/US2019/019075, filed on Feb. 22, 2019, which claims priority upon U.S. provisional application Ser. No. 62/634,251, filed Feb. 23, 2018, and 62/754,291, filed on Nov. 1, 2018. These applications are hereby incorporated by reference in their entireties for all their teachings.

### CROSS REFERENCE TO SEQUENCE LISTING

This application contains a sequence listing filed in ST.26 format entitled “930201-1101_Sequence_Listing.xml” created on May 15, 2023, and having a file size of 139,802 bytes. The content of the sequence listing is incorporated herein in its entirety.

### BACKGROUND

Exposure to UV radiation causes harmful effects in a wide variety of things, both living and non-living. For example, exposure of human skin to UV radiation can cause severe sunburn and skin cancer and exposure of beneficial microorganisms to UV radiation can kill them. UV radiation can also cause materials to degrade prematurely and thus suffer mechanical failure or otherwise become unable to serve their intended purpose.

The harmful effects of UV radiation can generally be prevented or lessened through the simple step of using a compound or composition to absorb all or a portion of the UV radiation before it reaches the item it may harm. For example, chemicals in sunscreen absorb a portion of the UV radiation that would normally reach the skin and, as a result, help protect the skin from sunburn and skin cancer.

Although numerous substances capable of absorbing UV radiation are known, not all of them are suitable for all possible uses. Further, some substances may be expensive to produce or may have harmful side effects, such as toxicity or undesired chemical reactions with a protected material. Other substances simply do not last long enough in the environment in which they are used, or persist long after their period of usefulness.

Microbial contamination of materials and surfaces can also cause a range of problems. Harmful microbes may cause infections in humans or animals. Other undesirable microorganisms can cause materials to degrade prematurely, can cause crop failure (e.g., due to fungal species) or food spoilage, can emit unpleasant odors, and the like. Antibiotics and antiseptic products have traditionally been used to control harmful microbial species; however, microorganisms often acquire antibiotic resistance after a few generations of exposure to these products and compounds. Additionally, many antibiotics have unpleasant side effects and many antiseptic products are particularly harsh to surfaces, such as, for example, human skin, or are toxic to wildlife or aquatic species. Furthermore, antibiotics or antiseptic products applied to the surface layer of an object may eventually wear or wash off, thus losing their effectiveness.

Accordingly, there is a demand for new substances able to absorb UV radiation. Furthermore, there is a demand for new antimicrobial substances. It is particularly desirable if both types of substance are biocompatible and biodegradable and further, if the same substances have simultaneously both anti-microbial and UV-protective properties. Additionally, it would be desirable if these substances could be incorporated to articles intended for consumer use such as, for example, food and beverage containers, thereby imparting their properties to these articles. The present invention addresses these needs.

### SUMMARY

Described herein are anti-microbial and UV-protective biological devices and extracts produced therefrom. The biological devices include microbial cells transformed with a DNA construct containing genes for producing proteins such as, for example, zinc-related protein/oxidase, silicatein, silaffin, and alcohol dehydrogenase. In some instances, the biological devices also include a gene for lipase. Methods for producing and using the devices are also described herein. Finally, compositions and methods for using the devices and extracts to kill microbial species or prevent microbial growth and to reduce or prevent UV-induced damage or exposure to materials, items, plants, and human and animal subjects are described herein. Also disclosed are biological devices producing polyactive carbohydrates and carbo sugars, as well as compositions and articles incorporating both extracts from these devices and the anti-microbial and UV-protective extracts.

The advantages to the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIGS. 1A and 1B show, respectively, a linear and circular schematic of a constructed plasmid showing the direction, placement, and size of genetic parts used of an exemplary DNA device for producing anti-microbial and UV-protective extracts as described herein.

FIGS. 2A and 2B show, respectively, a linear and circular schematic of a constructed plasmid showing the direction, placement, and size of genetic parts used of an alternative exemplary DNA device for producing anti-microbial and UV-protective extracts as described herein.

FIGS. 3A and 3B show, respectively, a linear and circular schematic of a constructed plasmid showing the direction, placement, and size of genetic parts used of an exemplary DNA device for producing a polyactive carbohydrate as described herein.

FIGS. 4A and 4B show, respectively, a linear and circular schematic of a constructed plasmid showing the direction, placement, and size of genetic parts used of an alternative exemplary DNA device for producing a polyactive carbohydrate as described herein.

FIG. 5A shows a schematic of a constructed pYES2 plasmid showing the direction, placement, and size of genetic parts used in one aspect of the DNA device.

5B shows a schematic of a constructed pYES2 plasmid showing the direction, placement, and size of genetic parts used in one aspect of the DNA device.

FIGS. 5C and 5D show, respectively, a linear and circular schematic of a constructed pYES2 plasmid showing the direction, placement, and size of genetic parts used of an exemplary DNA device for producing carbo sugars described herein.

FIGS. 6A and 6B show, respectively, a linear and circular schematic of a constructed plasmid showing the direction, placement, and size of genetic parts used of an alternative exemplary DNA device for producing carbo sugars as described herein.

FIGS. 7A and 7B show, respectively, a linear and circular schematic of a constructed plasmid showing the direction, placement, and size of genetic parts used of an alternative exemplary DNA device for producing carbo sugars as described herein.

FIG. 8 shows a sample cup prepared from the biofoams disclosed herein.

FIG. 9 shows a glucosamine calibration curve used to determine the concentration of polyactive carbohydrate in culture extracts.

FIGS. 10A, 10B, and 10C show, respectively: plastic glued to plastic, wood glued to wood, metal glued to metal using adhesives produced using the extracts and according to the methods disclosed herein.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “a plasmid” includes mixtures of two or more such plasmids, and the like.

“Optional” or “optionally” means that the subsequently described event or circumstance can or cannot occur, and that the description includes instances where the event or circumstance occurs and instances where it does not. For example, the phrase “optionally includes a reporter protein” means that the reporter protein may or may not be present.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Admixing” or “admixture” refers to a combination of two or more components together wherein there is no chemical reaction or physical interaction. The terms “admixing” and “admixture” can also include the chemical reaction or physical interaction between any of the components described herein upon mixing to produce the composition. The components can be admixed alone, in water, in another solvent, or in a combination of solvents.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed that while specific reference to each various individual and collective combination and permutation of these compounds may not be explicitly disclosed, each is specifically contemplated and described herein. For example, if a yeast is disclosed and discussed and a number of different compatible yeast plasmids are discussed, each and every combination and permutation of yeast and yeast plasmid that is possible is specifically contemplated unless specifically indicated to the contrary. For example, if a class of molecules A, B, and C are disclosed as well as a class of molecules D, E, and F, and an example of a combination molecule, A-D, is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the subgroup of A-E, B-F, and C-E is specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed, it is understood that each of these additional steps can be performed with any specific embodiment or combination of embodiments of the disclosed methods, and that each such combination is specifically contemplated and should be considered disclosed.

References in the specification and concluding claims to parts by weight, of a particular element or component in a composition or article, denote the weight relationship between the element or component and any other elements or components in the composition or article for which a part by weight is expressed. Thus, in a compound containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Described herein is a process for producing anti-microbial and/or UV-protective extracts that includes (a) making a DNA construct containing genes for a zinc related protein/oxidase, silicatein, silaffin, alcohol dehydrogenase II, and, optionally, lipase; (b) introducing the DNA construct into host microbial cells via transformation or transfection; and (c) culturing the microbial cells to produce anti-microbial and UV-protective extracts. Also described herein are processes for producing carbo sugars that include (a) making a DNA construct containing genes for cellulose synthase, galactomannan galactosyltransferase, and, optionally, lipase; (b) introducing the DNA construct into host microbial cells via transformation or transfection; and (c) culturing the microbial cells to produce carbo sugars. Further, described herein are processes for producing polyactive carbohydrates that include (a) making a DNA construct containing genes for chitin synthase, chitosanase, chitin deacetylase, and, optionally, lipase; (b) introducing the DNA construct into host microbial cells via transformation or transfection; and (c) culturing the microbial cells to produce polyactive carbohydrates.

### I. DNA Constructs

DNA constructs are provided herein for the production of anti-microbial and UV-protective proteins, polyactive carbohydrates, carbo sugars, extracts, and other components. It is understood that one way to define the variants and derivatives of the genetic components and DNA constructs described herein is in terms of homology/identity to specific known sequences. Those of skill in the art readily understand how to determine the homology of two nucleic acids. For example, the homology can be calculated after aligning the two sequences so that the homology is at its highest level. Another way of calculating homology can be performed according to published algorithms (see Zuker, M., Science, 244:48-52, 1989; Jaeger et al., Proc. Natl. Acad. Sci. USA, 86:7706-7710, 1989; Jaeger et al., Methods Enzymol., 183:281-306, 1989, which are herein incorporated by reference for at least material related to nucleic acid alignment).

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of mutations and homology can be combined together in any combination, such as embodiments that have at least 70%, 75%, 80%, 85%, 90%, 95%, or 99% homology to a particular sequence wherein the variants are conservative or silent mutations. It is understood that any of the sequences described herein can be a variant or derivative having the homology values listed above.

In one aspect, a database such as, for example, GenBank, can be used to determine the sequences of genes and/or regulatory regions of interest, the species from which these elements originate, and related homologous sequences.

In one aspect, provided herein is a DNA construct comprising the following genetic components:
a) a gene that expresses zinc-related protein/oxidase;
b) a gene that expresses silicatein;
c) a gene that expresses silaffin; and
d) a gene that expresses alcohol dehydrogenase II.

In one aspect, provided herein is a DNA construct comprising the following genetic components:
a) a gene that expresses zinc-related protein/oxidase;
b) a gene that expresses silicatein;
c) a gene that expresses silaffin; and
d) a gene that expresses alcohol dehydrogenase II; and
e) a gene that expresses lipase.

In another aspect, provided herein is a DNA construct comprising the following genetic components:
a) a gene that expresses chitin synthase;
b) a gene that expresses chitosanase; and
c) a gene that expresses chitin deacetylase.

In still another aspect, provided herein is a DNA construct comprising the following genetic components:
a) a gene that expresses chitin synthase;
b) a gene that expresses chitosanase;
c) a gene that expresses chitin deacetylase; and
d) a gene that expresses lipase.

In yet another aspect, provided herein is a DNA construct comprising the following genetic components:
a) a gene that expresses cellulose synthase; and
b) a gene that expresses galactomannan galactosyltransferase.

Each component of the DNA construct is described in detail below.

In one aspect, the nucleic acids (e.g., genes that express zinc-related protein/oxidase, silicatein, silaffin, alcohol dehydrogenase II, lipase, chitin synthase, chitosanase, chitin deacetylase, cellulose synthase, and galactomannan galactosyltransferase) used in the DNA constructs described herein can be amplified using polymerase chain reaction (PCR) prior to being ligated into a plasmid or other vector. Typically, PCR-amplification techniques make use of primers, or short, chemically-synthesized oligonucleotides that are complementary to regions on each respective strand flanking the DNA or nucleotide sequence to be amplified. A person having ordinary skill in the art will be able to design or choose primers based on the desired experimental conditions. In general, primers should be designed to provide for both efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the skilled practitioner with primer design. Primers can optionally incorporate restriction enzyme recognition sites at their 5′ ends to assist in later ligation into plasmids or other vectors.

PCR can be carried out using purified DNA, unpurified DNA that is integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or genes. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In a further aspect, the plasmid can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of that coding sequence.

As used herein, “expression” refers to transcription and/or accumulation of an mRNA derived from a gene or DNA fragment. Expression may also be used to refer to translation of mRNA into a peptide, polypeptide, or protein.

In one aspect, the zinc-related protein/oxidase is a member of or is related to the family of proteins known as FAD/NAD(P)-binding oxidoreductases. In a further aspect, activation of zinc-related protein can occur in times of oxidative stress to cells. In another aspect, the zinc-related protein/oxidase is calmodulin or another zinc-binding protein, or a homolog thereof. In one aspect, the zinc-related protein binds zinc (e.g., zinc metal or ions). In another aspect, the zinc-related protein is involved in the oxidation of zinc metal to zinc ions (e.g., Zn⁺²).

In one aspect, the gene that expresses zinc-related protein is isolated from an animal. In a further aspect, the animal is a fish such as, for example, Atlantic salmon. In an alternative aspect, the gene that expresses zinc-related protein is isolated from a bacterium. In one aspect, the bacterium is a Streptomyces, Polaribacter, Kitasatospora, Actinobacter, Azospirillum, Clostridium, Collimonas, or Micromonospora species. In a still further aspect, the gene that expresses zinc-related protein is isolated from an alga. In one aspect, the alga is Guillardia theta. In a further aspect, the gene that expresses zinc-related protein/oxidase has SEQ ID NO. 1 or at least 70% homology thereto, at least 75% homology thereto, at least 80% homology thereto, at least 85% homology thereto, at least 90% homology thereto, or at least 95% homology thereto. In one aspect, the gene that expresses zinc-related protein/oxidase is isolated from Streptomyces zinciresistens and can be found in GenBank with GI number EGX59011.1.

Other sequences expressing zinc-related protein/oxidase or related or homologous genes can be identified in a database such as, for example, GenBank. In one aspect, sequences useful herein include those with the GI numbers listed in Table 1.

TABLE 1
Zinc-Related Protein/Oxidase Genes

| | Sequence | |
|---|---|---|
| Source Organism | Description | GI Number |
| Streptomyces lincolnensis | genomic DNA | CP016438.1 |
| Streptomyces sp. 4F | genomic DNA | CP013142.1 |
| Streptomyces collinus | genomic DNA | CP006259.1 |
| Streptomyces avermitilis | genomic DNA | BA000030.4 |
| Streptomyces sp. 3124.6 | genomic DNA | LT670819.1 |
| Streptomyces sp. 1H-SSA4 | genomic DNA | CP022161.1 |
| Streptomyces parvulus | genomic DNA | CP015866.1 |
| Streptomyces ambofaciens | genomic DNA | CP012949.1 |
| Streptomyces ambofaciens | genomic DNA | CP012382.1 |
| Streptomyces scabiei | genomic DNA | FN554889.1 |
| Streptomyces davawensis | genomic DNA | HE971709.1 |
| Polaribacter sp. SA4-12 | genomic DNA | CP019334.1 |
| Streptomyces sp. 11-1-2 | genomic DNA | CP022545.1 |
| Streptomyces sp. CdTB01 | genomic DNA | CP013743.1 |
| Kitasatospora setae | genomic DNA | AP010968.1 |
| Streptomyces pluripotens | genomic DNA | CP022433.1 |
| Streptomyces pluripotens | genomic DNA | CP021080.1 |
| Streptomyces pactum | genomic DNA | CP019724.1 |
| Polaribacter sp. Hel1 33 78 | genomic DNA | LT629794.1 |
| Streptomyces sp. TLI_053 | genomic DNA | LT629775.1 |
| Streptomyces pactum | genomic DNA | CP016795.1 |
| Streptomyces puniscabiei | genomic DNA | CP017248.1 |
| Streptomyces griseochromogenes | genomic DNA | CP016279.1 |
| Streptomyces incarnatus | genomic DNA | CP011497.1 |
| Kitasatospora aureofaciens | genomic DNA | CP020567.1 |
| Streptomyces sp. S10(2016) | genomic DNA | CP015098.1 |
| Streptomyces reticuli | genomic DNA | LN997842.1 |
| Actinobacteria bacterium | genomic DNA | CP015603.1 |
| IMCC25003 | | |
| Polaribacter sp. KT25b | genomic DNA | LT629752.1 |
| Streptomyces hygroscopicus | genomic DNA | CP013219.1 |
| subsp. limoneus | | |
| Streptomyces sp. Mg1 | genomic DNA | CP011664.1 |
| Azospirillum brasilense | genomic DNA | CP007796.1 |
| Streptomyces hygroscopicus | genomic DNA | CP003720.1 |
| subsp. Jinggangensis | | |
| Streptomyces hygroscopicus | genomic DNA | CP003275.1 |
| subsp. Jinggangensis | | |
| Streptomyces sp. 2323.1 | genomic DNA | LT907981.1 |
| Clostridium cochlearium | genomic DNA | LT906477.1 |
| Collimonas arenae | genomic DNA | CP009962.1 |
| Polaribacter sp. MED152 | genomic DNA | CP004349.1 |
| Streptomyces sp. S8 | genomic DNA | CP015362.1 |
| Micromonospora echinofusca | genomic DNA | LT607733.1 |
| Streptomyces sp. PBH53 | genomic DNA | CP011799.1 |
| Streptomyces fulvissimus | genomic DNA | CP005080.1 |
| Streptomyces katrae | genomic DNA | CP020042.1 |
| Streptomyces silaceus | genomic DNA | CP015588.1 |
| Streptomyces venezuelae | genomic DNA | CP018074.1 |
| Salmo salar | putative | XM_014213459.1 |
| | calmodulin | |
| Streptomyces venezuelae | genomic DNA | FR845719.1 |
| Salmo salar | putative | BT059493.1 |
| | calmodulin | |
| Salmo salar | putative | BT045544.1 |
| | calmodulin | |
| Streptomyces albireticuli | genomic DNA | CP021744.1 |
| Streptomyces sp. 3211 | genomic DNA | CP020039.1 |
| Clostridium sporogenes | genomic DNA | CP011663.1 |
| Clostridium sporogenes | genomic DNA | CP009225.1 |
| Clostridium botulinum | genomic DNA | CP006902.1 |
| Guillardia theta | genomic DNA | XM_005830304.1 |

Silicateins are biosilica-forming enzymes. A typical silicatein uses a monomeric silicon compound as substrate. Silicateins further display proteolytic activity similar to that of cathepsin L, a lysosomal endopeptidase found in humans and other mammals, birds, fish, and invertebrates such as sponges.

In one aspect, the gene that expresses silicatein is isolated from a freshwater or marine sponge such as, for example, Suberites domuncula, Hymeniacidon perlevis, Tethya aurantium, Latrunculia oparinae, Mycale phyllophila, Ephydatia fluviatilis, Geodia cydonium, Spongilla lacustris, Lubomirskia baicalensis, Baikalospongia intermedia, Halochondria okadai, Ephydatia muelleri, or another sponge. In another aspect, the gene that expresses silicatein also has cathepsin L activity. Further in this aspect, the gene is isolated from a coral such as, for example, Orbicella faveolata. In another aspect, the gene is isolated from an echinoderm such as Acanthaster planci or a mollusk such as Crassotrea gigas or Crassotrea virginica. In yet another aspect, the gene is isolated from a fish such as, for example, Ictalurus furcatus. In one aspect, the gene that expresses silicatein is isolated from Suberites domuncula and can be found in GenBank with GI number AJ272013.1.

In another aspect, the gene that expresses silicatein has SEQ ID NO. 2 or at least 70% homology thereto, at least 75% homology thereto, at least 80% homology thereto, at least 85% homology thereto, at least 90% homology thereto, or at least 95% homology thereto.

Other sequences expressing silicatein or related or homologous genes can e identified in a database such as, for example, GenBank. In one aspect, sequences useful herein include those with the GI numbers listed in Table 2.

TABLE 2
Silicatein Genes

| | Sequence | |
|---|---|---|
| Source Organism | Description | GI Number |
| Suberites domuncula | silicatein | AJ272013.1 |
| Suberites domuncula | silicatein | HG964669.1 |
| Hymeniacidon perlevis | silicatein | DQ364228.1 |
| Tethya aurantium | silicatein | FR748155.1 |
| Tethya aurantium | silicatein | FR748154.1 |
| Suberites domuncula | silicatein | AJ877017.1 |
| Tethya aurantia | silicatein | AF032117.1 |
| Suberites domuncula | silicatein | HG964668.1 |
| Latrunculia oparinae | silicatein | EU888269.1 |
| Tethya aurantia | silicatein | AF098670.1 |
| Hymeniacidon perlevis | silicatein | EF174599.1 |
| Latrunculia oparinae | silicatein | EU909156.1 |
| Mycale phyllophila | silicatein | KX197954.1 |
| Ephydatia fluviatilis | silicatein | AB370207.1 |
| Latrunculia oparinae | silicatein | EU909155.1 |
| Geodia cydonium | silicatein | AM500857.1 |
| Spongilla lacustris | silicatein | AM941430.1 |
| Ephydatia fluviatilis | silicatein | AB370210.1 |
| Lubomirskia baicalensis | silicatein | AJ968947.1 |
| Autosaccus sp. GV-2009 | silicatein-like | GQ387054.1 |
| | protein | |
| Spongilla lacustris | silicatein | AM941429.1 |
| Ephydatia sp. n. 1 | silicatein | FM160557.1 |
| PW-2008 | | |
| Ephydatia fluviatilis | silicatein | AB370206.1 |
| Lubomirskia baicalensis | silicatein | AJ872183.1 |
| Ephydatia sp. n. 2 | silicatein | FM160562.1 |
| PW-2008 | | |
| Baikalospongia | silicatein | FM160556.1 |
| intermedia | | |
| Lubomirskia baicalensis | silicatein | AJ968945.1 |
| Spongilla lacustris | silicatein | AM941431.1 |
| Halichondira okadai | silicatein | AB071667.1 |
| Ephydatia muelleri | silicatein | FM160558.1 |
| Ephydatia fluviatilis | silicatein | AB219573.1 |
| Latrunculia oparinae | silicatein | EU909158.1 |
| Ephydatia sp. n. 2 | silicatein | FM160561.1 |
| PW-2008 | | |
| Ephydatia muelleri | silicatein | FM160559.1 |
| Ephydatia fluviatilis | silicatein | AB370208.1 |
| Amphimedon | cathepsin L1-like | XM_003383678.3 |
| queenslandica | | |
| Latrunculia oparinae | silicatein | EU909157.1 |
| Acanthodendrilla sp. | silicatein | FJ013044.1 |
| Vietnam | | |
| Discodermia japonica | silicatein | FR748157.1 |
| Anoplophora | cathepsin L1-like | XM_018720387.1 |
| glabripennis | | |
| Baikalospongia | silicatein | FM160555.1 |
| intermedia | | |
| Lubomirskia baicalensis | silicatein | AJ968946.1 |
| Ephydatia muelleri | silicatein | FM160560.1 |
| Ephydatia fluviatilis | silicatein | AM167901.1 |
| Lubomirskia baicalensis | silicatein | AJ786771.1 |
| Swartschewskia | silicatein | FM160565.1 |
| papyracea | | |
| Ephydatia fluviatilis | silicatein | AM167900.1 |
| Spongilla lacustris | silicatein | GU289406.1 |
| Orbicella faveolata | cathepsin L1-like | XM_020775559.1 |
| Ictalurus furcatus | cathepsin L | GU588322.1 |
| Acanthaster planci | cathepsin L1-like | XM_022226328.1 |
| Amphimedon | cathepsin L1-like | XM_003383677.3 |
| queenslandica | | |
| Crassotrea gigas | cathepsin L1 | XM_011448189.2 |
| Apostichopus japonicas | cathepsin L | KM523550.1 |
| Acanthodendrilla sp. | silicatein | FJ013043.1 |
| Vietnam | | |
| Crassostrea virginica | cathepsin L1-like | XM_022469675.1 |
| Crassostrea virginica | cathepsin L1-like | XM_022469674.1 |
| Crassostrea virginica | cathepsin L1-like | XM_022469673.1 |
| Crassostrea virginica | cathepsin L1-like | XM_022469672.1 |
| Branchiostoma floridae | hypothetical protein | XM_002611705.1 |
| Oreochromis niloticus | digestive cysteine | XM_019348535.1 |
| | proteinase 2-like | |
| Oreochromis niloticus | cathepsin L1 | XM_019348480.1 |
| Oreochromis niloticus | cathepsin L1 | XM_019348479.1 |
| Branchiostoma floridae | hypothetical protein | XM_002611707.1 |
| Astyanax mexicanus | cathepsin L1-like | XM_007241546.3 |
| Crassostrea virginica | cathepsin L1-like | XM_022471536.1 |
| Crassostrea virginica | cathepsin L1-like | XM_022471535.1 |
| Meriones unquiculatus | cathepsin L1-like | XM_021650453.1 |
| Branchiostoma belcheri | cathepsin L1-like | XM_019767779.1 |
| Pygocentrus nattereri | digestive cysteine | XM_017688979.1 |
| | proteinase 2-like | |
| Vigna angularis | senescence-specific | XM_017577302.1 |
| | cysteine protease | |
| | SAG39-like | |
| Ictalurus punctatus | cathepsin L1-like | XM_017475948.1 |
| Ictalurus punctatus | cathepsin L1-like | XM_017475947.1 |
| Baikalospongia | silicatein | HQ668149.1 |
| fungiformis | | |
| Lubomirskia baicalensis | silicatein | GU222667.1 |
| Crassostrea virginica | cathepsin L1-like | XM_022464354.1 |
| Cricetulus griseus | cathepsin L1 | XM_007621048.2 |
| Cricetulus griseus | cathepsin L1 | XM_007607734.2 |
| Biomphalaria glabrata | cathepsin L1-like | XM_013205493.1 |
| Fragaria vesca | senescence-specific | XM_004305390.2 |
| | cysteine protease | |
| | SAG39-like | |
| Crassostrea virginica | cathepsin L1-like | XM_022466099.1 |
| Crassostrea virginica | cathepsin L1-like | XM_022466098.1 |
| Chrysochloris asiatica | cathepsin L1-like | XM_006872586.1 |
| Ephydatia fluviatilis | silicatein | GU289405.1 |
| Acanthamoeba | cysteine protease | EF053509.1 |
| castellanii | | |
| Branchiostoma | cathepsin | AY333297.1 |
| lanceolatum | | |
| Orbicella faveolata | cathepsin L1-like | XM_020776462.1 |
| Pygocentrus nattereri | cathepsin L1-like | XM_017723497.1 |
| Pygocentrus nattereri | cathepsin L1-like | XM_017723496.1 |
| Lubomirskia baicalensis | silicatein | GU222669.1 |
| Ephydatia muelleri | silicatein | GU289408.1 |
| N. norvegicus | cathepsin L | X80990.1 |
| Lubomirskia baikalensis | silicatein | AJ877018.1 |
| Mizuhopecten yessoensis | cathepsin L1-like | XM_021491465.1 |
| Bactrocera dorsalis | cathepsin L | XM_019992690.1 |
| Branchiostoma belcheri | cathepsin L1-like | XM_019767758.1 |
| Branchiostoma belcheri | cathepsin L1-like | XM_019767757.1 |
| Branchiostoma belcheri | cathepsin L1-like | XM_019767756.1 |
| Branchiostoma belcheri | cathepsin L1-like | XM_019767755.1 |
| Anoplophora | cathepsin L1-like | XM_018720390.1 |
| glabripennis | proteinase | |

Silaffins are proteins expressed by diatoms and are involved in cell wall formation in those organisms. Silaffins can precipitate silica in various forms on the nano- and micro-scales. Silaffins may act as organic matrices for the genesis of biosilica and the structure, including pore size, of the resultant biosilica can be affected by the presence of other molecules such as phosphate, nitrogen, polyamines, peptides, and so forth.

In one aspect, the gene that expresses a silaffin is isolated from a diatom such as, for example, Cylindrotheca fusiformis, In another aspect, the gene that expresses a silaffin is isolated from a fungus such as, for example, Kazachstania naganishii, Cyberlindnera jadinii, Verticilium dahliae. In yet another aspect, the gene that expresses a silaffin is isolated from a bacterium such as, for example, Streptomyces hygroscopicus or a Pseudomonas species or another unicellular organism such as Capsaspora owczarzaki. In an alternative aspect, the gene is isolated from an insect such as, for example, Drosophila navojoa or Drosophila busckii. In one aspect, the gene that expresses a silaffin is isolated from a plant such as Citrus sinensis or Citrus clementina. In still another aspect, the gene that expresses a silaffin is isolated from a fish such as, for example, Kryptolebias marmoratus, Austrufundulus limnaeus, or Oryzias latipes or from a mammal such as the bighorn sheep (Ovis canadensis canadensis), leopard (Panthera pardus), Siberian tiger (Panthera tigris altaica), cheetah (Acinonyx jubatus), rat (Rattus norvegicus), alpaca (Vicugna pacos), or domestic cat (Felis catus).

In a further aspect, the gene that expresses silaffin has SEQ ID NO. 3 or at least 70% homology thereto, at least 75% homology thereto, at least 80% homology thereto, at least 85% homology thereto, at least 90% homology thereto, or at least 95% homology thereto.

Other sequences expressing silaffin or related or homologous genes can e identified in a database such as, for example, GenBank. In one aspect, sequences useful herein include those with the GI numbers listed in Table 3.

TABLE 3
Silaffin Genes

| Source Organism | Sequence Description | GI Number |
|---|---|---|
| Cylindrotheca | silaffin | AF191634.1 |
| fusiformis | | |
| Kazachstania | hypothetical protein | XM_022610325.1 |
| naganishii | | |
| Kryptolebias | involucrin-like | XM_017428128.1 |
| marmoratus | | |
| Kazachstania | chromosome 11 sequence | HE978324.1 |
| naganishii | | |
| Cyberlindnera jadinii | hypothetical protein | XM_020217246.1 |
| Streptomyces | genomic DNA | CP018627.1 |
| hygroscopicus | | |
| Ovis canadensis | chromosome 5 sequence | CP011890.1 |
| canadensis | | |
| Verticillium dahlia | chromosome 2 sequence | CP010981.1 |
| Capsaspora owczarzaki | proprotein convertase | XM_011272068.1 |
| | subtilisin/kexin type 1 | |
| Capsaspora owczarzaki | proprotein convertase | XM_004347923.2 |
| | subtilisin/kexin type 1 | |
| Verticilium dahlia | hypothetical protein | XM_009652892.1 |
| Verticilium dahlia | chromosome 1 sequence | CP009075.1 |
| Oryzias latipes | chromosome 1 sequence | CP020621.1 |
| Kryptolebias | myeloid/lymphoid or | XM_017431390.1 |
| marmoratus | mixed-lineage leukemia | |
| Austrofundulus | afadin-like | XM_013999827.1 |
| limnaeus | | |
| Austrofundulus | afadin-like | XM_013999825.1 |
| limnaeus | | |
| Austrufundulus | afadin-like | XM_014026841.1 |
| limnaeus | | |
| Felis catus | CD48 transcript variant | XM_019822316.1 |
| Felis catus | CD48 transcript variant | XM_006943036.3 |
| Felix catus | CD48 transcript variant | XM_019822315.1 |
| Panthera pardus | CD48 transcript variant | XM_019433348.1 |
| Panthera pardus | CD48 transcript variant | XM_019433346.1 |
| Panthera pardus | CD48 transcript variant | XM_019433345.1 |
| Drosophila navojoa | condensing complex subunit 1 | XM_018106778.1 |
| Panthera tigris altaica | CD48 molecule | XM_007092375.2 |
| Citrus sinensis | DEAD-box ATP-dependent | XM_015529892.1 |
| | RNA helicase 21 | |
| Citrus sinensis | DEAD-box ATP-dependent | XM_006476625.2 |
| | RNA helicase 21 | |
| Vicugna pacos | interleukin 4 receptor transcript | XM_006201225.2 |
| | variant | |
| Vicugna pacos | interleukin 4 receptor transcript | XM_015236831.1 |
| | variant | |
| Acinonyx jubatus | CD48 molecule | XM_015076211.1 |
| Austrofundulus | repetitive proline-rich cell wall | XM_014032218.1 |
| limnaeus | protein 2-like | |
| Drosophila busckii | chromosome 2R sequence | CP012524.1 |
| Pseudomonas sp. MT-1 | genomic DNA | AP014655.1 |
| Citrus clementina | hypothetical protein | XM_006439663.1 |
| Rattus norvegicus | secretoglobin family 3A | NM_001013180.1 |

In one aspect, the gene that expresses alcohol dehydrogenase II catalyzes the conversion of ethanol to acetaldehyde. In another aspect, alcohol dehydrogenase II can act with any one of a number of primary unbranched aliphatic alcohols. In some aspects, alcohol dehydrogenase II requires at least two Zn²⁺ ions per subunit to function. In other aspects, one molecule of NAD⁺ is required to convert an alcohol to an aldehyde or ketone using alcohol dehydrogenase II.

In one aspect, the gene that expresses alcohol dehydrogenase II is isolated from a fungus. In a further aspect, the fungus is a yeast such as, for example, Saccharomyces cerevisiae. In a still further aspect, the S. cerevisiae is from strain S288c, N85, Y12, YSR127, AHY0914, YJM451, YJM470, YJM554, YJM555, YJM682, YJM689, YJM972, YJM975, YJM978, YJM996, YJM1083, YJM1133, YJM1190, YJM1208, YJM1250, YJM1307, YJM1356, YJM1381, YJM1383, YJM1385, YJM1386, YJM1388, YJM1389, YJM1419, YJM1433, YJM1456, YJM1460, YJM1526, YJM1592, or YJM1615. In an alternative aspect, the S. cerevisiae is a wild-type strain. In a further aspect, the gene that expresses alcohol dehydrogenase II has SEQ ID NO. 4 or at least 70% homology thereto, at least 75% homology thereto, at least 80% homology thereto, at least 85% homology thereto, at least 90% homology thereto, or at least 95% homology thereto. In one aspect, the gene that expresses alcohol dehydrogenase II is isolated from Saccharomyces cerevisiae and can be found in GENBank with GI number J01314.1.

In a further aspect, the gene that expresses alcohol dehydrogenase II has SEQ ID NO. 4 or at least 70% homology thereto, at least 75% homology thereto, at least 80% homology thereto, at least 85% homology thereto, at least 90% homology thereto, or at least 95% homology thereto.

Other sequences expressing alcohol dehydrogenase II or related or homologous genes can e identified in a database such as, for example, GenBank. In one aspect, sequences useful herein include those with the GI numbers listed in Table 4.

TABLE 4
Alcohol Dehydrogenase Genes

| Source Organism | Sequence Description | GI Number |
|---|---|---|
| Saccharomyces cerevisiae | alcohol dehydrogenase II | J01314.1 |
| Saccharomyces cerevisiae | chromosome XIII sequence | CP005453.2 |
| Saccharomyces cerevisiae | chromosome XIII sequence | CP020135.1 |
| Saccharomyces cerevisiae | chromosome XIII sequence | CP005452.2 |
| Saccharomyces cerevisiae | chromosome XIII sequence | CP005450.2 |
| Saccharomyces cerevisiae | chromosome XIII sequence | BK006946.2 |
| Saccharomyces cerevisiae | alcohol dehydrogenase II | NM_001182812.1 |
| Saccharomyces cerevisiae | chromosome XIII sequence | Z49212.1 |
| Saccharomyces cerevisiae | synthetic construct | EF059086.1 |
| Saccharomyces cerevisiae | alcohol dehydrogenase | M38457.1 |
| Saccharomyces cerevisiae | chromosome XIII sequence | CP005464.2 |
| Saccharomyces cerevisiae | chromosome XIII sequence | CP005483.2 |
| Saccharomyces cerevisiae | chromosome XIII sequence | CP005432.2 |
| Saccharomyces cerevisiae | chromosome XIII sequence | CP005482.2 |
| Saccharomyces cerevisiae | chromosome XIII sequence | LN907796.1 |
| Saccharomyces cerevisiae | chromosome XIII sequence | CP005456.1 |
| Saccharomyces cerevisiae | chromosome XIII sequence | CP005455.1 |
| Saccharomyces cerevisiae | chromosome XIII sequence | CP005440.1 |
| Saccharomyces cerevisiae | chromosome XIII sequence | CP020203.1 |
| Saccharomyces cerevisiae | chromosome XIII sequence | CP005403.2 |
| Saccharomyces cerevisiae | chromosome XIII sequence | CP005472.2 |
| Saccharomyces cerevisiae | chromosome XIII sequence | CP005465.2 |
| Saccharomyces cerevisiae | chromosome XIII sequence | CP005405.2 |
| Saccharomyces cerevisiae | chromosome XIII sequence | CP005414.2 |
| Saccharomyces cerevisiae | chromosome XIII sequence | CP005412.2 |
| Saccharomyces cerevisiae | chromosome XIII sequence | CP005451.2 |
| Saccharomyces cerevisiae | chromosome XIII sequence | CP011559.1 |
| Saccharomyces cerevisiae | chromosome XIII sequence | CP005436.1 |
| Saccharomyces cerevisiae | chromosome XIII sequence | CP005426.1 |
| Saccharomyces cerevisiae | chromosome XIII sequence | CP005406.1 |
| Saccharomyces cerevisiae | glucose-repressible alcohol | KJ137141.1 |
| | dehydrogenase II | |
| Saccharomyces cerevisiae | alcohol dehydrogenase | JX901290.1 |
| Saccharomyces cerevisiae | chromosome XIII sequence | CP008010.1 |
| Saccharomyces cerevisiae | chromosome XIII sequence | CP020169.1 |
| Saccharomyces cerevisiae | chromosome XIII sequence | CP005449.2 |
| Saccharomyces cerevisiae | chromosome XIII sequence | CP005429.2 |
| Saccharomyces cerevisiae | chromosome XIII sequence | CP005419.2 |
| Saccharomyces cerevisiae | chromosome XIII sequence | CP005409.2 |
| Saccharomyces cerevisiae | chromosome XIII sequence | CP005428.2 |
| Saccharomyces cerevisiae | chromosome XIII sequence | CP005418.2 |
| Saccharomyces cerevisiae | chromosome XIII sequence | CP005408.2 |
| Saccharomyces cerevisiae | chromosome XIII sequence | CP005477.2 |
| Saccharomyces cerevisiae | chromosome XIII sequence | CP005417.2 |
| Saccharomyces cerevisiae | chromosome XIII sequence | CP005425.2 |
| Saccharomyces cerevisiae | chromosome XIII sequence | CP008265.1 |
| Saccharomyces cerevisiae | chromosome XIII sequence | CP008367.1 |
| Saccharomyces cerevisiae | chromosome XIII sequence | CP008554.1 |
| Saccharomyces cerevisiae | chromosome XIII sequence | CP008537.1 |
| Saccharomyces cerevisiae | chromosome XIII sequence | CP008520.1 |
| Saccharomyces cerevisiae | chromosome XIII sequence | CP008129.1 |
| Saccharomyces cerevisiae | chromosome XIII sequence | CP007993.1 |
| Saccharomyces cerevisiae | chromosome XIII sequence | CP005444.2 |
| Saccharomyces cerevisiae | chromosome XIII sequence | CP005434.2 |
| Saccharomyces cerevisiae | chromosome XIII sequence | CP005424.2 |
| Saccharomyces cerevisiae | chromosome XIII sequence | CP005404.2 |
| Saccharomyces cerevisiae | chromosome XIII sequence | CP005423.2 |
| Saccharomyces cerevisiae | chromosome XIII sequence | CP005422.2 |
| Saccharomyces cerevisiae | chromosome XIII sequence | CP005402.2 |
| Saccharomyces cerevisiae | chromosome XIII sequence | CP005421.2 |
| Saccharomyces cerevisiae | chromosome XIII sequence | CP005411.2 |
| Saccharomyces cerevisiae | chromosome XIII sequence | CP005420.2 |
| Saccharomyces cerevisiae | chromosome XIII sequence | CP005427.1 |
| Saccharomyces cerevisiae | chromosome XIII sequence | CP005416.1 |
| Saccharomyces cerevisiae | chromosome XIII sequence | CP005475.2 |
| Saccharomyces cerevisiae | chromosome XIII sequence | CP008401.1 |
| Saccharomyces cerevisiae | chromosome XIII sequence | CP008503.1 |
| Saccharomyces cerevisiae | chromosome XIII sequence | CP005398.2 |
| Saccharomyces cerevisiae | chromosome XIII sequence | CP005478.2 |
| Saccharomyces cerevisiae | chromosome XIII sequence | CP005437.2 |
| Saccharomyces cerevisiae | chromosome XIII sequence | CP005407.2 |
| Saccharomyces cerevisiae | chromosome XIII sequence | CP005454.2 |
| Saccharomyces cerevisiae | chromosome XIII sequence | CP005462.2 |
| Saccharomyces cerevisiae | chromosome XIII sequence | CP005461.2 |
| Saccharomyces cerevisiae | chromosome XIII sequence | CP005401.2 |
| Saccharomyces cerevisiae | chromosome XIII sequence | CP005396.1 |
| Saccharomyces cerevisiae | glucose-repressible alcohol | KJ137139.1 |
| | dehydrogenase II | |
| Saccharomyces cerevisiae | chromosome XIII sequence | CP005479.2 |
| Saccharomyces cerevisiae | chromosome XIII sequence | CP005469.2 |
| Saccharomyces cerevisiae | chromosome XIII sequence | CP005399.2 |
| Saccharomyces cerevisiae | chromosome XIII sequence | CP005397.2 |
| Saccharomyces cerevisiae | chromosome XIII sequence | CP005415.2 |
| Saccharomyces cerevisiae | chromosome XIII sequence | CP005395.2 |
| Saccharomyces cerevisiae | chromosome XIII sequence | CP008248.1 |
| Saccharomyces cerevisiae | chromosome XIII sequence | CP008333.1 |
| Saccharomyces cerevisiae | chromosome XIII sequence | CP008316.1 |
| Saccharomyces cerevisiae | chromosome XIII sequence | CP008299.1 |
| Saccharomyces cerevisiae | chromosome XIII sequence | CP008282.1 |
| Saccharomyces cerevisiae | chromosome XIII sequence | CP008231.1 |
| Saccharomyces cerevisiae | chromosome XIII sequence | CP008418.1 |
| Saccharomyces cerevisiae | chromosome XIII sequence | CP008384.1 |
| Saccharomyces cerevisiae | chromosome XIII sequence | CP008350.1 |
| Saccharomyces cerevisiae | chromosome XIII sequence | CP008486.1 |
| Saccharomyces cerevisiae | chromosome XIII sequence | CP008469.1 |
| Saccharomyces cerevisiae | chromosome XIII sequence | CP008435.1 |
| Saccharomyces cerevisiae | chromosome XIII sequence | CP008588.1 |
| Saccharomyces cerevisiae | chromosome XIII sequence | CP008571.1 |
| Saccharomyces cerevisiae | chromosome XIII sequence | CP008656.1 |
| Saccharomyces cerevisiae | chromosome XIII sequence | CP008639.1 |
| Saccharomyces cerevisiae | chromosome XIII sequence | CP008605.1 |
| Saccharomyces cerevisiae | chromosome XIII sequence | CP008214.1 |

A lipase is an esterase that catalyzes the hydrolysis of fats, oils, and lipids. In one aspect, the gene that expresses lipase is isolated from a bacterium. In a further aspect, the bacterium is a Micrococcus species, a Pseudomonas species, a Moraxella species, or an Acinetobacter species. In a further aspect, the gene that expresses lipase has SEQ ID NO. 7 or at least 70% homology thereto, at least 75% homology thereto, at least 80% homology thereto, at least 85% homology thereto, at least 90% homology thereto, or at least 95% homology thereto. In one aspect, the gene that expresses lipase can be positioned anywhere in the DNA construct disclosed herein. In one aspect, the gene that expresses lipase is positioned 5′ (i.e., prior) to the gene that expresses zinc related protein/oxidase.

Other sequences expressing lipase or related or homologous genes can be identified in a database such as, for example, GenBank. In one aspect, sequences useful herein include those with the GI numbers listed in Table 5:

TABLE 5
Lipase Genes

| Source Organism | Sequence Description | GI Number |
|---|---|---|
| Micrococcus sp. HL-2003 | lipase gene | AY268069.1 |
| Pseudomonas sp. | esterase gene | M68491.1 |
| Moraxella L1 | lipase 1 | X53053.1 |
| A. calcoaceticus | carboxylesterase and | X74839.1 |
| | peptidyl prolyl-cis- | |
| | trans-isomerase | |
| Acinetobacter sp. ADP1 | genomic DNA | CR543861.1 |
| A. calcoaceticus | esterase | X71598.1 |
| Pseudomonas trivialis | genomic DNA | CP011507.1 |
| Pseudomonas azotoformans | genomic DNA | CP019856.1 |
| Pseudomonas | genomic DNA | LT629689.1 |
| extremaustralis | | |
| Pseudomonas fluorescens | genomic DNA | CP005975.1 |
| Pseudomonas fluorescens | genomic DNA | CP010896.1 |
| Pseudomonas fluorescens | genomic DNA | AF228666.1 |
| Pseudomonas simiae | genomic DNA | CP007637.1 |
| Pseudomonas fluorescens | genomic DNA | AM181176.4 |
| Pseudomonas Antarctica | genomic DNA | CP015600.1 |
| Pseudomonas fluorescens | genomic DNA | CP015639.1 |
| Pseudomonas fluorescens | genomic DNA | LT907842.1 |
| Pseudomonas sp. NS1 | genomic DNA | CP022960.1 |
| Pseudomonas poae | genomic DNA | LT629706.1 |
| Pseudomonas poae | genomic DNA | CP004045.1 |
| Pseudomonas rhodesiae | genomic DNA | LT629801.1 |
| Pseudomonas trivialis | genomic DNA | LT629760.1 |
| Pseudomonas azotoformans | genomic DNA | LT629702.1 |
| Pseudomonas Antarctica | genomic DNA | LT629704.1 |
| Pseudomonas fluorescens | genomic DNA | CP012400.1 |
| Pseudomonas azotoformans | genomic DNA | CP014546.1 |
| Pseudomonas yamanorum | genomic DNA | LT629793.1 |
| Pseudomonas prosekii | genomic DNA | LT629762.1 |
| Pseudomonas koreensis | genomic DNA | CP014947.1 |
| Pseudomonas libanensis | genomic DNA | LT629699.1 |
| Pseudomonas sp. GR 6-02 | genomic DNA | CP011567.1 |
| Pseudomonas fluorescens | genomic DNA | CP014868.1 |
| Pseudomonas fluorescens | genomic DNA | CP011117.1 |
| Pseudomonas fluorescens | genomic DNA | S69066.1 |
| Pseudomonas cedrina | genomic DNA | LT629753.1 |
| Pseudomonas sp. bs2935 | genomic DNA | LT629744.1 |
| Pseudomonas fluorescens | genomic DNA | CP017296.1 |
| Pseudomonas sp. WCS374 | genomic DNA | CP007638.1 |
| Pseudomonas fluorescens | genomic DNA | CP003041.1 |
| Pseudomonas corrugate | genomic DNA | LT629798.1 |
| Pseudomonas corrugate | genomic DNA | CP014262.1 |
| Pseudomonas mediterranea | genomic DNA | LT629790.1 |
| Pseudomonas tolaasii | genomic DNA | CP020369.1 |
| Pseudomonas fluorescens | genomic DNA | CP015638.1 |
| Pseudomonas fluorescens | genomic DNA | CP015637.1 |
| Pseudomonas sp. TKP | genomic DNA | CP006852.1 |
| Synthetic construct | carboxylesterase | HM212419.1 |
| Synthetic construct | carboxylesterase | FJ213454.1 |
| Pseudomonas sp. | genomic DNA | CP023969.1 |
| FDAARGOS 380 | | |
| Pseudomonas synxantha | genomic DNA | LT629786.1 |
| Pseudomonas orientalis | genomic DNA | LT629782.1 |
| Pseudomonas sp. | genomic DNA | LN854573.1 |
| URMO17WK12:I11 | | |

Chitin synthase is a glycosyltransferase enzyme that catalyzes the following reaction:

UDP-N-acetyl-D-glucosamine+[1,4-(N-acetyl-β-D-glucosaminyl)]ₙ→UDP+[1,4-(N-acetyl-β-D-glucosaminyl)]ₙ₊₁
where UDP is uridine diphosphate and N-acetyl-D-glucosamine units are added to the growing chitin chain one residue at a time.

In one aspect, the gene that expresses chitin synthase is isolated from yeast. In a further aspect, the yeast can be Saccharomyces cerevisiae. In a still further aspect, the S. cerevisiae strain that is the source of chitin synthase can be strain S288c, BSPX042, ySR127, DBVPG6765, YJM1526, YJM972, YJM969, YJM470, YJM248, YJM1478, YJM996, YJM244, YJM1477, YJM1387, YJM993, YJM1332, YJM1242, YJM990, T63, T52, or any other commonly cultured experimental strain of yeast. In another aspect, the S. cerevisiae is a wild type strain. In a further aspect, the gene that expresses chitin synthase has SEQ ID NO. 6 or at least 70% homology thereto, at least 75% homology thereto, at least 80% homology thereto, at least 85% homology thereto, at least 90% homology thereto, or at least 95% homology thereto. In one aspect, the gene that expresses chitin synthase is isolated from Saccharomyces cerevisiae and can be found in GenBank with GI number NC_001146.8.

Other sequences expressing chitin synthase or related or homologous genes can be identified in a database such as, for example, GenBank. In one aspect, sequences useful herein include those with the GI numbers listed in Table 6.

TABLE 6
Chitin Synthase Genes

| Source Organism | Sequence Description | GI Number |
|---|---|---|
| Saccharomyces cerevisiae | synthetic construct | DQ331902.1 |
| Saccharomyces cerevisiae | chromosome XIV sequence | CP020136.1 |
| Saccharomyces cerevisiae | chromosome XIV sequence | CP014729.1 |
| Saccharomyces cerevisiae | chromosome XIV sequence | CP011560.1 |
| Saccharomyces cerevisiae | chromosome XIV sequence | BK006947.3 |
| Saccharomyces cerevisiae | chitin synthase | NM_001183030.2 |
| Saccharomyces cerevisiae | chromosome XIV sequence | Z71468.1 |
| Saccharomyces cerevisiae | chitin synthase | M14045.1 |
| Saccharomyces cerevisiae | chromosome XIV sequence | CP020170.1 |
| Saccharomyces cerevisiae | chromosome XIV sequence | CP005579.2 |
| Saccharomyces cerevisiae | chromosome XIV sequence | CP005519.2 |
| Saccharomyces cerevisiae | chromosome XIV sequence | CP005518.2 |
| Saccharomyces cerevisiae | chromosome XIV sequence | CP005508.2 |
| Saccharomyces cerevisiae | chromosome XIV sequence | CP005498.2 |
| Saccharomyces cerevisiae | chromosome XIV sequence | CP005577.2 |
| Saccharomyces cerevisiae | chromosome XIV sequence | CP005527.2 |
| Saccharomyces cerevisiae | chromosome XIV sequence | CP005497.2 |
| Saccharomyces cerevisiae | chromosome XIV sequence | CP005576.2 |
| Saccharomyces cerevisiae | chromosome XIV sequence | CP005556.2 |
| Saccharomyces cerevisiae | chromosome XIV sequence | CP005526.2 |
| Saccharomyces cerevisiae | chromosome XIV sequence | CP005545.2 |
| Saccharomyces cerevisiae | chromosome XIV sequence | CP005535.2 |
| Saccharomyces cerevisiae | chromosome XIV sequence | CP005525.2 |
| Saccharomyces cerevisiae | chromosome XIV sequence | CP008334.1 |
| Saccharomyces cerevisiae | chromosome XIV sequence | CP008317.1 |
| Saccharomyces cerevisiae | chromosome XIV sequence | CP008351.1 |
| Saccharomyces cerevisiae | chromosome XIV sequence | CP008470.1 |
| Saccharomyces cerevisiae | chromosome XIV sequence | CP008572.1 |
| Saccharomyces cerevisiae | chromosome XIV sequence | CP008674.1 |
| Saccharomyces cerevisiae | chromosome XIV sequence | CP008657.1 |
| Saccharomyces cerevisiae | chromosome XIV sequence | CP008623.1 |
| Saccharomyces cerevisiae | chromosome XIV sequence | CP008181.1 |
| Saccharomyces cerevisiae | chromosome XIV sequence | CP008147.1 |
| Saccharomyces cerevisiae | chromosome XIV sequence | CP008045.1 |
| Saccharomyces cerevisiae | chromosome XIV sequence | CP008028.1 |
| Saccharomyces cerevisiae | chromosome XIV sequence | CP008011.1 |
| Saccharomyces cerevisiae | chromosome XIV sequence | CP007926.1 |
| Saccharomyces cerevisiae | chromosome XIV sequence | CP007892.1 |
| Saccharomyces cerevisiae | chromosome XIV sequence | CP007875.1 |
| Saccharomyces cerevisiae | chromosome XIV sequence | CP007858.1 |
| Saccharomyces cerevisiae | chromosome XIV sequence | CP007824.1 |
| Saccharomyces cerevisiae | chromosome XIV sequence | CP005494.2 |
| Saccharomyces cerevisiae | chromosome XIV sequence | CP005583.2 |
| Saccharomyces cerevisiae | chromosome XIV sequence | CP005573.2 |
| Saccharomyces cerevisiae | chromosome XIV sequence | CP005533.2 |
| Saccharomyces cerevisiae | chromosome XIV sequence | CP005523.2 |
| Saccharomyces cerevisiae | chromosome XIV sequence | CP005503.2 |
| Saccharomyces cerevisiae | chromosome XIV sequence | CP005552.2 |
| Saccharomyces cerevisiae | chromosome XIV sequence | CP005542.2 |
| Saccharomyces cerevisiae | chromosome XIV sequence | CP005532.2 |
| Saccharomyces cerevisiae | chromosome XIV sequence | CP005522.2 |
| Saccharomyces cerevisiae | chromosome XIV sequence | CP005521.2 |
| Saccharomyces cerevisiae | chromosome XIV sequence | CP005550.2 |
| Saccharomyces cerevisiae | chromosome XIV sequence | CP005520.2 |
| Saccharomyces cerevisiae | chromosome XIV sequence | CP005500.2 |
| Saccharomyces cerevisiae | chromosome XIV sequence | CP005524.1 |
| Saccharomyces cerevisiae | chromosome XIV sequence | CP005516.1 |
| Saccharomyces cerevisiae | chromosome XIV sequence | CP004112.1 |
| Saccharomyces cerevisiae | chromosome XIV sequence | CP008266.1 |
| Saccharomyces cerevisiae | chromosome XIV sequence | CP008453.1 |
| Saccharomyces cerevisiae | chromosome XIV sequence | CP008521.1 |
| Saccharomyces cerevisiae | chromosome XIV sequence | CP008640.1 |
| Saccharomyces cerevisiae | chromosome XIV sequence | CP008079.1 |
| Saccharomyces cerevisiae | chromosome XIV sequence | CP005529.2 |
| Saccharomyces cerevisiae | chromosome XIV sequence | CP005499.2 |
| Saccharomyces cerevisiae | chromosome XIV sequence | CP005548.2 |
| Saccharomyces cerevisiae | chromosome XIV sequence | CP005547.2 |
| Saccharomyces cerevisiae | chromosome XIV sequence | CP005546.2 |
| Saccharomyces cerevisiae | chromosome XIV sequence | CP005536.2 |
| Saccharomyces cerevisiae | chromosome XIV sequence | CP005506.2 |
| Saccharomyces cerevisiae | chromosome XIV sequence | CP008283.1 |
| Saccharomyces cerevisiae | chromosome XIV sequence | CP008232.1 |
| Saccharomyces cerevisiae | chromosome XIV sequence | CP008487.1 |
| Saccharomyces cerevisiae | chromosome XIV sequence | CP008436.1 |
| Saccharomyces cerevisiae | chromosome XIV sequence | CP008589.1 |
| Saccharomyces cerevisiae | chromosome XIV sequence | CP008555.1 |
| Saccharomyces cerevisiae | chromosome XIV sequence | CP008538.1 |
| Saccharomyces cerevisiae | chromosome XIV sequence | CP008606.1 |
| Saccharomyces cerevisiae | chromosome XIV sequence | CP008198.1 |
| Saccharomyces cerevisiae | chromosome XIV sequence | CP008164.1 |
| Saccharomyces cerevisiae | chromosome XIV sequence | CP008691.1 |
| Saccharomyces cerevisiae | chromosome XIV sequence | CP008130.1 |
| Saccharomyces cerevisiae | chromosome XIV sequence | CP008113.1 |
| Saccharomyces cerevisiae | chromosome XIV sequence | CP008096.1 |
| Saccharomyces cerevisiae | chromosome XIV sequence | CP007977.1 |
| Saccharomyces cerevisiae | chromosome XIV sequence | CP007960.1 |
| Saccharomyces cerevisiae | chromosome XIV sequence | CP005564.2 |
| Saccharomyces cerevisiae | chromosome XIV sequence | CP005544.2 |
| Saccharomyces cerevisiae | chromosome XIV sequence | CP005563.2 |
| Saccharomyces cerevisiae | chromosome XIV sequence | CP005581.2 |
| Saccharomyces cerevisiae | chromosome XIV sequence | CP005551.2 |
| Saccharomyces cerevisiae | chromosome XIV sequence | CP005531.2 |
| Saccharomyces cerevisiae | chromosome XIV sequence | CP005580.2 |
| Saccharomyces cerevisiae | chromosome XIV sequence | CP005530.2 |
| Saccharomyces cerevisiae | chromosome XIV sequence | CP005510.2 |
| Saccharomyces cerevisiae | chromosome XIV sequence | FN393086.1 |
| Saccharomyces cerevisiae | chromosome XIV sequence | CP008504.1 |
| Saccharomyces cerevisiae | chromosome XIV sequence | CP007909.1 |
| Saccharomyces cerevisiae | chromosome XIV sequence | CP005549.2 |
| Saccharomyces cerevisiae | chromosome XIV sequence | CP005539.2 |

Chitosanase is any one of a class of enzymes that perform hydrolysis of β-(1→4)-linkages between D-glucosamine residues in a partially acetylated chitosan molecule. The hydrolysis carried out by chitosanase typically occurs in the middle of the chitosan rather than at the ends.

In one aspect, the gene that expresses chitosanase is isolated from yeast. In a further aspect, the yeast can be Saccharomyces cerevisiae. In another aspect, the S. cerevisiae strain can be S288c, BSPX042, ySR127, YJM683, YJM682, YJM554, YJM541, YJM456, YJM326, YJM1615, YJM1208, YJM1133, NCIM3107, NCIM3186, T52, T63, YJM1573, YJM1402, YJM1401, another commonly cultured experimental strain, or can be a wild type strain. In a further aspect, the gene that expresses chitosanase has SEQ ID NO. 7 or at least 70% homology thereto, at least 75% homology thereto, at least 80% homology thereto, at least 85% homology thereto, at least 90% homology thereto, or at least 95% homology thereto. In one aspect, the gene that expresses chitosanase is isolated from Saccharomyces cerevisiae and can be found in GenBank with GI number AAB67331.1.

Other sequences expressing chitosanase or related or homologous genes can be identified in a database such as, for example, GenBank. In one aspect, sequences useful herein include those with the GI numbers listed in Table 7.

TABLE 7
Chitosanase Genes

| Source Organism | Sequence Description | GI Number |
|---|---|---|
| Saccharomyces cerevisiae | chromosome XII sequence | CP020134.1 |
| Saccharomyces cerevisiae | chromosome XII sequence | CP014727.1 |
| Saccharomyces cerevisiae | chromosome XII sequence | CP011558.1 |
| Saccharomyces cerevisiae | chromosome XII sequence | CP006456.1 |
| Saccharomyces cerevisiae | chromosome XII sequence | CP006455.1 |
| Saccharomyces cerevisiae | chromosome XII sequence | CP006451.1 |
| Saccharomyces cerevisiae | chromosome XII sequence | CP006450.1 |
| Saccharomyces cerevisiae | chromosome XII sequence | CP006448.1 |
| Saccharomyces cerevisiae | chromosome XII sequence | CP006443.1 |
| Saccharomyces cerevisiae | chromosome XII sequence | CP006434.1 |
| Saccharomyces cerevisiae | chromosome XII sequence | CP006383.1 |
| Saccharomyces cerevisiae | chromosome XII sequence | CP006379.1 |
| Saccharomyces cerevisiae | chromosome XII sequence | BK006945.2 |
| Saccharomyces cerevisiae | genomic DNA | NM_001182173.1 |
| Saccharomyces cerevisiae | chromosome XII sequence | U17243.1 |
| Saccharomyces cerevisiae | endochitinase | M74070.1 |
| Saccharomyces cerevisiae | chromosome XII sequence | CP009950.1 |
| Saccharomyces cerevisiae | chromosome XII sequence | CP011821.1 |
| Saccharomyces cerevisiae | chromosome XII sequence | CP008196.1 |
| Saccharomyces cerevisiae | chromosome XII sequence | CP008553.1 |
| Saccharomyces cerevisiae | chromosome XII sequence | CP008536.1 |
| Saccharomyces cerevisiae | chromosome XII sequence | CP008519.1 |
| Saccharomyces cerevisiae | chromosome XII sequence | CP008655.1 |
| Saccharomyces cerevisiae | chromosome XII sequence | CP008213.1 |
| Saccharomyces cerevisiae | chromosome XII sequence | CP008315.1 |
| Saccharomyces cerevisiae | chromosome XII sequence | CP008264.1 |
| Saccharomyces cerevisiae | chromosome XII sequence | CP008145.1 |
| Saccharomyces cerevisiae | chromosome XII sequence | CP006431.1 |
| Saccharomyces cerevisiae | chromosome XII sequence | CP006411.1 |
| Saccharomyces cerevisiae | chromosome XII sequence | CP006410.1 |
| Saccharomyces cerevisiae | chromosome XII sequence | CP008247.1 |
| Saccharomyces cerevisiae | chromosome XII sequence | CP008366.1 |
| Saccharomyces cerevisiae | chromosome XII sequence | CP008349.1 |
| Saccharomyces cerevisiae | chromosome XII sequence | CP008485.1 |
| Saccharomyces cerevisiae | chromosome XII sequence | CP008604.1 |
| Saccharomyces cerevisiae | chromosome XII sequence | CP008179.1 |
| Saccharomyces cerevisiae | chromosome XII sequence | CP008162.1 |
| Saccharomyces cerevisiae | chromosome XII sequence | CP008128.1 |
| Saccharomyces cerevisiae | chromosome XII sequence | CP008026.1 |
| Saccharomyces cerevisiae | chromosome XII sequence | CP007975.1 |
| Saccharomyces cerevisiae | chromosome XII sequence | CP007958.1 |
| Saccharomyces cerevisiae | chromosome XII sequence | CP007941.1 |
| Saccharomyces cerevisiae | chromosome XII sequence | CP007873.1 |
| Saccharomyces cerevisiae | chromosome XII sequence | CP007856.1 |
| Saccharomyces cerevisiae | chromosome XII sequence | CP007839.1 |
| Saccharomyces cerevisiae | chromosome XII sequence | CP008400.1 |
| Saccharomyces cerevisiae | chromosome XII sequence | CP020219.1 |
| Saccharomyces cerevisiae | chromosome XII sequence | CP008451.1 |
| Saccharomyces cerevisiae | chromosome XII sequence | CP006417.1 |
| Saccharomyces cerevisiae | chromosome XII sequence | CP006398.1 |
| Saccharomyces cerevisiae | chromosome XII sequence | CP006389.1 |
| Saccharomyces cerevisiae | chromosome XII sequence | CP008332.1 |
| Saccharomyces cerevisiae | chromosome XII sequence | CP008298.1 |
| Saccharomyces cerevisiae | chromosome XII sequence | CP008417.1 |
| Saccharomyces cerevisiae | chromosome XII sequence | CP008434.1 |
| Saccharomyces cerevisiae | chromosome XII sequence | CP008587.1 |
| Saccharomyces cerevisiae | chromosome XII sequence | CP008638.1 |
| Saccharomyces cerevisiae | chromosome XII sequence | CP008689.1 |
| Saccharomyces cerevisiae | chromosome XII sequence | CP008111.1 |
| Saccharomyces cerevisiae | chromosome XII sequence | CP008094.1 |
| Saccharomyces cerevisiae | chromosome XII sequence | CP008077.1 |
| Saccharomyces cerevisiae | chromosome XII sequence | CP008043.1 |
| Saccharomyces cerevisiae | chromosome XII sequence | CP007924.1 |
| Saccharomyces cerevisiae | chromosome XII sequence | CP007907.1 |
| Saccharomyces cerevisiae | chromosome XII sequence | CP007890.1 |
| Saccharomyces cerevisiae | chromosome XII sequence | CP007822.1 |
| Saccharomyces cerevisiae | chromosome XII sequence | CP008281.1 |
| Saccharomyces cerevisiae | chromosome XII sequence | CP006429.1 |
| Saccharomyces cerevisiae | chromosome XII sequence | CP006419.1 |
| Saccharomyces cerevisiae | chromosome XII sequence | CP008230.1 |
| Saccharomyces cerevisiae | chromosome XII sequence | CP008468.1 |
| Saccharomyces cerevisiae | chromosome XII sequence | CP008672.1 |
| Saccharomyces cerevisiae | chromosome XII sequence | CP008502.1 |
| Saccharomyces cerevisiae | chromosome XII sequence | CP006454.1 |
| Saccharomyces cerevisiae | chromosome XII sequence | CP006445.1 |
| Saccharomyces cerevisiae | chromosome XII sequence | CP006427.1 |
| Saccharomyces cerevisiae | chromosome XII sequence | CP006420.1 |
| Saccharomyces cerevisiae | chromosome XII sequence | CP006409.1 |
| Saccharomyces cerevisiae | chromosome XII sequence | CP006390.1 |
| Saccharomyces cerevisiae | chromosome XII sequence | CP008621.1 |
| Saccharomyces cerevisiae | chromosome XII sequence | CP008009.1 |
| Saccharomyces cerevisiae | chromosome XII sequence | CP006452.1 |
| Saccharomyces cerevisiae | chromosome XII sequence | CP006430.1 |
| Saccharomyces cerevisiae | chromosome XII sequence | CP006401.1 |
| Saccharomyces cerevisiae | chromosome XII sequence | CP006393.1 |
| Saccharomyces cerevisiae | chromosome XII sequence | CP007992.1 |
| Saccharomyces cerevisiae | chromosome XII sequence | CP006414.1 |
| Saccharomyces cerevisiae | chromosome XII sequence | CP008570.1 |
| Saccharomyces cerevisiae | chromosome XII sequence | CP008383.1 |
| Saccharomyces cerevisiae | chromosome XII sequence | CP020236.1 |
| Saccharomyces cerevisiae | chromosome XII sequence | CP006421.1 |
| Saccharomyces cerevisiae | chromosome XII sequence | CP008060.1 |
| Saccharomyces cerevisiae | chromosome XII sequence | CP020151.1 |
| Saccharomyces cerevisiae | chromosome XII sequence | CP006453.1 |
| Saccharomyces cerevisiae | chromosome XII sequence | CP006418.1 |
| Saccharomyces cerevisiae | chromosome XII sequence | CP006386.1 |
| Saccharomyces cerevisiae | chromosome XII sequence | CP006449.1 |
| Saccharomyces cerevisiae | chromosome XII sequence | CP006404.1 |
| Saccharomyces cerevisiae | chromosome XII sequence | LN907795.1 |
| Saccharomyces cerevisiae | chromosome XII sequence | CP006387.1 |

Chitin deacetylase is an enzyme that catalyzes the hydrolysis of chitin to chitosan and acetate. In one aspect, the chitin deacetylase reaction can proceed to completion. In an alternative aspect, the hydrolysis is incomplete, leaving some acetate groups attached to glucosamine residues in the polymer backbone.

In one aspect, the gene that expresses chitin deacetylase is isolated from yeast. In a further aspect, the yeast can be Saccharomyces cerevisiae. In another aspect, the S. cerevisiae strain can be Y12, S288c, BSPX042, N85, YJM470, YJM456, YJM1615, YJM1592, YJM1549, YJM1460, YJM1389, YJM1388, YJM1387, YJM1304, YJM1208, YJM689, YJM1202, YJM1199, YJM1133, YJM1381, YPS128, another commonly cultured experimental strain, or can be a wild type strain. In a further aspect, the gene that expresses chitin deacetylase has SEQ ID NO. 8 or at least 70% homology thereto, at least 75% homology thereto, at least 80% homology thereto, at least 85% homology thereto, at least 90% homology thereto, or at least 95% homology thereto. In one aspect, the gene that expresses chitin deacetylase is isolated from Saccharomyces cerevisiae and can be found in GenBank with accession number NM 001182196.

Other sequences expressing chitin deacetylase or related or homologous genes can be identified in a database such as, for example, GenBank. In one aspect, sequences useful herein include those with the GI numbers listed in Table 8.

TABLE 8
Chitin Deacetylase Genes

| Source Organism | Sequence Description | GI Number |
|---|---|---|
| Saccharomyces cerevisiae | chromosome XII sequence | CP020202.1 |
| Saccharomyces cerevisiae | chromosome XII sequence | CP020134.1 |
| Saccharomyces cerevisiae | chromosome XII sequence | CP014727.1 |
| Saccharomyces cerevisiae | chromosome XII sequence | LN907795.1 |
| Saccharomyces cerevisiae | chromosome XII sequence | CP006449.1 |
| Saccharomyces cerevisiae | chromosome XII sequence | CP006448.1 |
| Saccharomyces cerevisiae | chromosome XII sequence | CP006434.1 |
| Saccharomyces cerevisiae | chromosome XII sequence | CP006433.1 |
| Saccharomyces cerevisiae | chromosome XII sequence | CP006430.1 |
| Saccharomyces cerevisiae | chromosome XII sequence | CP006423.1 |
| Saccharomyces cerevisiae | chromosome XII sequence | CP006407.1 |
| Saccharomyces cerevisiae | chromosome XII sequence | CP006406.1 |
| Saccharomyces cerevisiae | chromosome XII sequence | CP006405.1 |
| Saccharomyces cerevisiae | chromosome XII sequence | CP006390.1 |
| Saccharomyces cerevisiae | chromosome XII sequence | CP006383.1 |
| Saccharomyces cerevisiae | chromosome XII sequence | BK006945.2 |
| Saccharomyces cerevisiae | chitin deacetylase | NM_001182196.1 |
| Saccharomyces cerevisiae | chromosome XII sequence | U17247.1 |
| Saccharomyces cerevisiae | chromosome XII sequence | CP006457.1 |
| Saccharomyces cerevisiae | chromosome XII sequence | CP006382.1 |
| Saccharomyces cerevisiae | chromosome XII sequence | CP006381.1 |
| Saccharomyces cerevisiae | chromosome XII sequence | CP006379.1 |
| Saccharomyces cerevisiae | chromosome XII sequence | CP006401.1 |
| Saccharomyces cerevisiae | chromosome XII sequence | CP020219.1 |
| Saccharomyces cerevisiae | chromosome XII sequence | CP006427.1 |
| Saccharomyces cerevisiae | chromosome XII sequence | CP006426.1 |
| Saccharomyces cerevisiae | chromosome XII sequence | CP006422.1 |
| Saccharomyces cerevisiae | chromosome XII sequence | CP006419.1 |
| Saccharomyces cerevisiae | chromosome XII sequence | CP006417.1 |
| Saccharomyces cerevisiae | chromosome XII sequence | CP006409.1 |
| Saccharomyces cerevisiae | chromosome XII sequence | CP006389.1 |
| Saccharomyces cerevisiae | chromosome XII sequence | CP006377.1 |
| Saccharomyces cerevisiae | chromosome XII sequence | CP020236.1 |
| Saccharomyces cerevisiae | chromosome XII sequence | CP006429.1 |
| Saccharomyces cerevisiae | chromosome XII sequence | CP006421.1 |
| Saccharomyces cerevisiae | chromosome XII sequence | CP006420.1 |
| Saccharomyces cerevisiae | chromosome XII sequence | CP006414.1 |
| Saccharomyces cerevisiae | chromosome XII sequence | CP006410.1 |
| Saccharomyces cerevisiae | chromosome XII sequence | CP011821.1 |
| Saccharomyces cerevisiae | chromosome XII sequence | CP006458.1 |
| Saccharomyces cerevisiae | chromosome XII sequence | CP006456.1 |
| Saccharomyces cerevisiae | chromosome XII sequence | CP006455.1 |
| Saccharomyces cerevisiae | chromosome XII sequence | CP006451.1 |
| Saccharomyces cerevisiae | chromosome XII sequence | CP006450.1 |
| Saccharomyces cerevisiae | chromosome XII sequence | CP006446.1 |
| Saccharomyces cerevisiae | chromosome XII sequence | CP006445.1 |
| Saccharomyces cerevisiae | chromosome XII sequence | CP006443.1 |
| Saccharomyces cerevisiae | chromosome XII sequence | CP006442.1 |
| Saccharomyces cerevisiae | chromosome XII sequence | CP006404.1 |
| Saccharomyces cerevisiae | chromosome XII sequence | CP006436.1 |
| Saccharomyces cerevisiae | chromosome XII sequence | CP006431.1 |
| Saccharomyces cerevisiae | chromosome XII sequence | CP006415.1 |
| Saccharomyces cerevisiae | chromosome XII sequence | CP006411.1 |
| Saccharomyces cerevisiae | chromosome XII sequence | CP006408.1 |
| Saccharomyces cerevisiae | chromosome XII sequence | CP009950.1 |
| Saccharomyces cerevisiae | chromosome XII sequence | CP008196.1 |
| Saccharomyces cerevisiae | chromosome XII sequence | CP020151.1 |
| Saccharomyces cerevisiae | chromosome XII sequence | CP008247.1 |
| Saccharomyces cerevisiae | chromosome XII sequence | CP008332.1 |
| Saccharomyces cerevisiae | chromosome XII sequence | CP008315.1 |
| Saccharomyces cerevisiae | chromosome XII sequence | CP008298.1 |
| Saccharomyces cerevisiae | chromosome XII sequence | CP008281.1 |
| Saccharomyces cerevisiae | chromosome XII sequence | CP008264.1 |
| Saccharomyces cerevisiae | chromosome XII sequence | CP008230.1 |
| Saccharomyces cerevisiae | chromosome XII sequence | CP008417.1 |
| Saccharomyces cerevisiae | chromosome XII sequence | CP008383.1 |
| Saccharomyces cerevisiae | chromosome XII sequence | CP008366.1 |
| Saccharomyces cerevisiae | chromosome XII sequence | CP008349.1 |
| Saccharomyces cerevisiae | chromosome XII sequence | CP008502.1 |
| Saccharomyces cerevisiae | chromosome XII sequence | CP008485.1 |
| Saccharomyces cerevisiae | chromosome XII sequence | CP008468.1 |
| Saccharomyces cerevisiae | chromosome XII sequence | CP008451.1 |
| Saccharomyces cerevisiae | chromosome XII sequence | CP008434.1 |
| Saccharomyces cerevisiae | chromosome XII sequence | CP008587.1 |
| Saccharomyces cerevisiae | chromosome XII sequence | CP008570.1 |
| Saccharomyces cerevisiae | chromosome XII sequence | CP008553.1 |
| Saccharomyces cerevisiae | chromosome XII sequence | CP008536.1 |
| Saccharomyces cerevisiae | chromosome XII sequence | CP008519.1 |
| Saccharomyces cerevisiae | chromosome XII sequence | CP008672.1 |
| Saccharomyces cerevisiae | chromosome XII sequence | CP008655.1 |
| Saccharomyces cerevisiae | chromosome XII sequence | CP008638.1 |
| Saccharomyces cerevisiae | chromosome XII sequence | CP008621.1 |
| Saccharomyces cerevisiae | chromosome XII sequence | CP008604.1 |
| Saccharomyces cerevisiae | chromosome XII sequence | CP008213.1 |
| Saccharomyces cerevisiae | chromosome XII sequence | CP008179.1 |
| Saccharomyces cerevisiae | chromosome XII sequence | CP008162.1 |
| Saccharomyces cerevisiae | chromosome XII sequence | CP008689.1 |
| Saccharomyces cerevisiae | chromosome XII sequence | CP008145.1 |
| Saccharomyces cerevisiae | chromosome XII sequence | CP008128.1 |
| Saccharomyces cerevisiae | chromosome XII sequence | CP008111.1 |
| Saccharomyces cerevisiae | chromosome XII sequence | CP008094.1 |
| Saccharomyces cerevisiae | chromosome XII sequence | CP008077.1 |
| Saccharomyces cerevisiae | chromosome XII sequence | CP008060.1 |
| Saccharomyces cerevisiae | chromosome XII sequence | CP008043.1 |
| Saccharomyces cerevisiae | chromosome XII sequence | CP008026.1 |
| Saccharomyces cerevisiae | chromosome XII sequence | CP008009.1 |
| Saccharomyces cerevisiae | chromosome XII sequence | CP007975.1 |
| Saccharomyces cerevisiae | chromosome XII sequence | CP007958.1 |
| Saccharomyces cerevisiae | chromosome XII sequence | CP007941.1 |
| Saccharomyces cerevisiae | chromosome XII sequence | CP007924.1 |

In one aspect, the gene that expresses cellulose synthase is isolated from plants. In a different aspect, the gene that expresses cellulose synthase is isolated from algae. In one aspect, the algal species is a red algal species such as, for example, Pyropia yezoensis (also known as Porphyra yezoensis) or Griffithsia monilis. In a further aspect, the gene that expresses cellulose synthase has SEQ ID NO. 9 or at least 70% homology thereto, at least 75% homology thereto, at least 80% homology thereto, at least 85% homology thereto, at least 90% homology thereto, or at least 95% homology thereto. In a further aspect, the cellulose synthase is able to use mannose as a substrate instead of or in addition to glucose.

Other sequences expressing cellulose synthase or related or homologous genes can be identified in a database such as, for example, GenBank. In one aspect, sequences useful herein include those with the GI numbers listed in Table 9:

TABLE 9
Cellulose Synthase Genes

| Source Organism | Sequence Description | GI Number |
|---|---|---|
| Porphyra vezoensis | cellulose synthase | EU279853.1 |
| Porphyra vezoensis | cellulose synthase | EU279861.1 |
| Porphyra vezoensis | cellulose synthase | EU279857.1 |
| | pseudogene | |
| Porphyra vezoensis | cellulose synthase | EU279858.1 |
| | pseudogene | |
| Porphyra vezoensis | cellulose synthase | EU279854.1 |
| | pseudogene | |
| Porphyra vezoensis | cellulose synthase | EU279855.1 |
| | pseudogene | |
| Chondrus crispus | cellulose synthase | XM_005715532.1 |
| | family | |
| Porphyra vezoensis | cellulose synthase | EU279859.1 |
| | pseudogene | |
| Porphyra vezoensis | cellulose synthase | EU279856.1 |
| | pseudogene | |
| Porphyra vezoensis | cellulose synthase | EU279860.1 |
| | pseudogene | |
| Gelidiella liqulata | cellulose synthase | KT920245.1 |
| | catalytic subunit A | |
| Gelidiella acerosa | cellulose synthase | KT920242.1 |
| | catalytic subunit A | |
| Parviphycus albertanoae | cellulose synthase | KT920246.1 |
| | catalytic subunit A | |
| Parviphycus felicinii | cellulose synthase | KT920247.1 |
| | catalytic subunit A | |
| Gelidiella incrassata | cellulose synthase | KT920244.1 |
| | catalytic subunit A | |
| Pterocladiella melanoidea | cellulose synthase | KT920254.1 |
| | catalytic subunit A | |
| Griffithsia monilis | cellulose synthase A | GU563823.1 |
| Gelidiella fanii | cellulose synthase | KT920243.1 |
| | catalytic subunit A | |
| Pterocladia rectangularis | cellulose synthase | KT920196.1 |
| | catalytic subunit A | |
| Ptilophora mediterranea | cellulose synthase | KT920238.1 |
| | catalytic subunit A | |
| Gelidium pacificum | cellulose synthase | KT920227.1 |
| | catalytic subunit A | |
| Gelidium microdon | cellulose synthase | KT920223.1 |
| | catalytic subunit A | |
| Gelidium johnstonii | cellulose synthase | KT920222.1 |
| | catalytic subunit A | |
| Pterocladella bartletti | cellulose synthase | KT920250.1 |
| | catalytic subunit A | |
| Pterocladia lucida | cellulose synthase | KT920248.1 |
| | catalytic subunit A | |
| Gelidium declerckii | cellulose synthase | KT920214.1 |
| | catalytic subunit A | |
| Ptilophora | cellulose synthase | KT920240.1 |
| pterocladioides | catalytic subunit A | |
| Pterocladia lucida | cellulose synthase | KT920249.1 |
| | catalytic subunit A | |
| Gelidium | cellulose synthase | KT920195.1 |
| madagascariense | catalytic subunit A | |
| Gelidium sp. GHB-2012 | cellulose synthase | KT920237.1 |
| | catalytic subunit A | |
| Gelidium crinate | cellulose synthase | KT920212.1 |
| | catalytic subunit A | |
| Ptilophora scalaramosa | cellulose synthase | KT920241.1 |
| | catalytic subunit A | |
| Gelidium robustum | cellulose synthase | KT920234.1 |
| | catalytic subunit A | |
| Gelidium purpurascens | cellulose synthase | KT920231.1 |
| | catalytic subunit A | |
| Gelidium pusillum | cellulose synthase | KT920232.1 |
| | catalytic subunit A | |
| Gelidium indonesianum | cellulose synthase | KT920218.1 |
| | catalytic subunit A | |
| Callophyllis japonica | cellulose synthase | KT920257.1 |
| | catalytic subunit A | |
| Gelidium nudifrons | cellulose synthase | KT920225.1 |
| | catalytic subunit A | |
| Gelidium isabelae | cellulose synthase | KT920219.1 |
| | catalytic subunit A | |
| Pterocladiella beachiae | cellulose synthase | KT920251.1 |
| | catalytic subunit A | |
| Gelidium spinosum | cellulose synthase | KT920235.1 |
| | catalytic subunit A | |
| Gelidium prostratum | cellulose synthase | KT920229.1 |
| | catalytic subunit A | |
| Geladium minimum | cellulose synthase | KT920224.1 |
| | catalytic subunit A | |
| Gelidium sp. SMB-2011a | cellulose synthase | KT920216.1 |
| | catalytic subunit A | |
| Gelidium elegans | cellulose synthase | KT920215.1 |
| | catalytic subunit A | |
| Gelidium coulteri | cellulose synthase | KT920211.1 |
| | catalytic subunit A | |
| Gracilaria textorii | cellulose synthase | KT920258.1 |
| | catalytic subunit A | |
| Gelidium corneum | cellulose synthase | KT920210.1 |
| | catalytic subunit A | |
| Gelidium bernabei | cellulose synthase | KT920207.1 |
| | catalytic subunit A | |
| Gelidium abbottiorum | cellulose synthase | KT920204.1 |
| | catalytic subunit A | |
| Grateloupia asiatica | cellulose synthase | KT920259.1 |
| | catalytic subunit A | |
| Ptilophora prolifera | cellulose synthase | KT920239.1 |
| | catalytic subunit A | |
| Gelidium crispum | cellulose synthase | KT920213.1 |
| | catalytic subunit A | |
| Chondrus crispus | cellulose synthase | XM_005711895.1 |
| | family | |
| Gelidium rex | cellulose synthase | KT920233.1 |
| | catalytic subunit A | |
| Gelidium pulchellum | cellulose synthase | KT920230.1 |
| | catalytic subunit A | |
| Gelidium coreanum | cellulose synthase | KT920209.1 |
| | catalytic subunit A | |
| Gelidium capense | cellulose synthase | KT920208.1 |
| | catalytic subunit A | |
| Rhodymenia intricata | cellulose synthase | KT920260.1 |
| | catalytic subunit A | |
| Gelidium asperum | cellulose synthase | KT920205.1 |
| | catalytic subunit A | |
| Aphanta pachyrrhiza | cellulose synthase | KT920193.1 |
| | catalytic subunit A | |
| Pterocladella nana | cellulose synthase | KT920255.1 |
| | catalytic subunit A | |
| Gelidium ornamense | cellulose synthase | KT920226.1 |
| | catalytic subunit A | |
| Gelidium hommersandii | cellulose synthase | KT920217.1 |
| | catalytic subunit A | |
| Gelidium australe | cellulose synthase | KT920206.1 |
| | catalytic subunit A | |
| Pterocladiella | cellulose synthase | KT920252.1 |
| caerulescens | catalytic subunit A | |
| Gelidium jejuense | cellulose synthase | KT920221.1 |
| | catalytic subunit A | |
| Gelidium japonicum | cellulose synthase | KT920220.1 |
| | catalytic subunit A | |
| Acanthopeltis | cellulose synthase | KT920200.1 |
| longiramulosa | catalytic subunit A | |
| Capreolia implexa | cellulose synthase | KT920201.1 |
| | catalytic subunit A | |
| Acanthopeltis japonica | cellulose synthase | KT920199.1 |
| | catalytic subunit A | |
| Acanthopeltis hirsuta | cellulose synthase | KT920198.1 |
| | catalytic subunit A | |
| Acanthopeltis hirsuta | cellulose synthase | KT920197.1 |
| | catalytic subunit A | |
| Gelidium vagum | cellulose synthase | KT920236.1 |
| | catalytic subunit A | |
| Aphanta sp. GHB-2016 | cellulose synthase | KT920194.1 |
| | catalytic subunit A | |
| Pterocladelia capillacea | cellulose synthase | KT920253.1 |
| | catalytic subunit A | |
| Gelidium pristoides | cellulose synthase | KT920228.1 |
| | catalytic subunit A | |
| Pterocladelia tenuis | cellulose synthase | KT920256.1 |
| | catalytic subunit A | |
| Gelidium divaricatum | cellulose synthase | KT920202.1 |
| | catalytic subunit A | |
| Aphanomyces astaci | hypothetical protein | XM_009832014.1 |
| Aphanomyces invadans | hypothetical protein | XM_008862896.1 |
| Acanthamoeba castellanii | putative cellulose | XM_004335119.1 |
| str. Neff | synthase | |
| Phytophthora sojae | hypothetical protein | XM_009526171.1 |
| Phytophthora parasitica | hypothetical protein | XM_008915779.1 |
| Aphanomyces invadans | hypothetical protein | XM_008862899.1 |
| Saprolegnia diclina | hypothetical protein | XM_008616683.1 |
| Phytophthora infestans | putative cellulose | XM_002897169.1 |
| | synthase catalytic | |
| | subunit | |
| Phytophthora sojae | cellulose synthase 1 | EF563997.1 |
| Phytophthora infestans | cellulose synthase 3 | EF563995.1 |
| Jatropha curcas | sucrose transport protein | XM_012212213.2 |
| Jatropah curcas | sucrose transport protein | XM_012212212.2 |
| Jatropha curcas | sucrose transport protein | XM_012212211.2 |
| Jatropha curcas | sucrose transport protein | XM_012212210.2 |
| Jatropha curcas | sucrose transport protein | NM_001319920.1 |
| Saprolegnia parasitica | hypothetical protein | XM_012347851.1 |
| Protopolystoma | unidentified | LM730806.1 |
| xenopodis | | |
| Aphanomyces invadans | hypothetical protein | XM_008869048.1 |
| Uncultured bacterium | unidentified | JX649872.1 |
| A1Q1 | | |
| Plasmopara viticola | cellulose synthase 1 | GQ258973.1 |
| Pyrus x bretschneideri | H2 finger protein | XM_009348167.2 |
| | ATL20-like | |

In one aspect, the gene that expresses galactomannan galactosyltransferase is isolated from a plant. In one aspect, the galactomannan galactosyltransferase is able to catalyze the synthesis of bonds between an oligo- or poly-mannose backbone and pendant galactose moieties to produce carbo sugars. In a further aspect, the plant is Oryza japonica, Medicago truncatula, Glycine max, Trigonella foenum-graecum, Lotus japonicus, Senna occidentalis, Cucumis sativus, Fragaria vesca, or Cyamopsis tetragonoloba. In a further aspect, the galactomannan galactosyltransferase has SEQ ID NOs. 10-12 or at least 70% homology thereto. In a further aspect, the gene that expresses galactomannan galactosyltransferase has SEQ ID NO. 12 or at least 70% homology thereto, at least 75% homology thereto, at least 80% homology thereto, at least 85% homology thereto, at least 90% homology thereto, or at least 95% homology thereto.

Other sequences expressing galactomannan galactosyltransferase or related or homologous genes can be identified in a database such as, for example, GenBank. In one aspect, sequences useful herein include those with the GI numbers listed in Table 10:

TABLE 10
Galactomannan Galactosyltransferase Genes

| Source Organism | Sequence Description | GI Number |
|---|---|---|
| Cyamopsis tetragonoloba | galactomannan | AJ938067.1 |
| | galactosyltransferase | |
| Glycine max | galactomannan | XM_003525821.3 |
| | galactosyltransferase | |
| | 1-like | |
| Medicago truncatula | unidentified | AC140720.21 |
| Medicago truncatula | galactosyl transferase | XM_003608493.2 |
| Lotus japonicus | galactomannan | AJ567668.1 |
| | galactosyltransferase | |
| Lupinus angustifolius | galactomannan | XM_019567307.1 |
| | galactosyltransferase | |
| | 1-like | |
| Lupinus angustifolius | galactomannan | XM_019567302.1 |
| | galactosyltransferase | |
| | 1-like | |
| Trigonella foenum- | alpha | AJ245478.1 |
| graecum | galactosyltransferase | |
| Arachis ipaensis | galactomannan | XM_016346122.2 |
| | galactosyltransferase 1 | |
| Arachis ipaensis | galactomannan | XM_016346121.2 |
| | galactosyltransferase 1 | |
| Ziziphus jujuba | galactomannan | XM_016034856.1 |
| | galactosyltransferase | |
| | 1-like | |
| Ziziphus jujuba | galactomannan | XM_016034855.1 |
| | galactosyltransferase | |
| | 1-like | |
| Arachis duranensis | galactomannan | XM_016108311.2 |
| | galactosyltransferase 1 | |
| Arachis duranensis | galactomannan | XM_016108310.2 |
| | galactosyltransferase 1 | |
| Glycine max | galactomannan | XM_003539215.3 |
| | galactosyltransferase | |
| | 1-like | |
| Glycine max | galactomannan | XM_006590559.2 |
| | galactosyltransferase | |
| | 1-like | |
| Glycine max | unidentified | AC235306.1 |
| Glycine max | unidentified | AK245471.1 |
| Senna occidentalis | galactomannan | AJ938068.1 |
| | galactosyltransferase | |
| Phaseolus vulgaris | hypothetical protein | XM_007156638.1 |
| Glycine max | galactomannan | XM_003517306.3 |
| | galactosyltransferase | |
| Juglans regia | putative | XM_018958015.1 |
| | glycosyltransferase 7 | |
| Prunus mume | galactomannan | XM_008239498.2 |
| | galactosyltransferase | |
| | 1-like | |
| Prunus mume | galactomannan | XM_008239489.2 |
| | galactosyltransferase | |
| | 1-like | |
| Prunus persica | galactomannan | XM_007205133.2 |
| | galactosyltransferase 1 | |
| Iponoea nil | glycosyltransferase | XM_019317795.1 |
| | 6-like | |
| Vigna radiata var. | galactomannan | XM_014665934.1 |
| radiata | galactosyltransferase | |
| | 1-like | |
| Lupinus angustifolius | galactomannan | XM_019589398.1 |
| | galactosyltransferase | |
| | 1-like | |
| Lupinus angustifolius | galactomannan | XM_019589395.1 |
| | galactosyltransferase | |
| | 1-like | |
| Lupinus angustifolius | galactomannan | XM_019589394.1 |
| | galactosyltransferase | |
| | 1-like | |
| Nelumbo nucifera | putative | XM_010274767.2 |
| | glycosyltransferase 7 | |
| Vigna angularis | galactomannan | XM_017570547.1 |
| | galactosyltransferase | |
| | 1-like | |
| Vigna angularis var. | unidentified | AP015043.1 |
| angularis | | |
| Pyrus x bretschneideri | galactomannan | XM_009364320.2 |
| | galactosyltransferase | |
| Vitis vinifera | unidentified | AM447747.2 |
| Morus notabilis | galactomannan | XM_010108622.1 |
| | galactosyltransferase 1 | |
| Malus domestica | galactomannan | NM_001328775.1 |
| | galactosyltransferase | |
| | 1-like | |
| Malus x domestica | galactomannan | XM_008363913.2 |
| | galactosyltransferase | |
| | like | |
| Malus x domestica | u1-nidentified | AB627270.1 |
| Malus x domestica | unidentified | HM122522.1 |
| Cucumis sativus | galactomannan | XM_004141806.2 |
| | galactosyltransferase | |
| | 1-like | |
| Malus x domestica | galactomannan | XM_008349663.1 |
| | galactosyltransferase | |
| | 1-like | |
| Cajanus cajan | galactomannan | XM_020374857.1 |
| | galactosyltransferase | |
| | 1-like | |
| Cajanus cajan | galactomannan | XM_020374856.1 |
| | galactosyltransferase | |
| | 1-like | |
| Capsicum annum | putative | XM_016705445.1 |
| | glycosyltransferase 7 | |
| Malus x domestica | unidentified | HM122524.1 |
| Fragaria vesca | galactomannan | XM_004288217.2 |
| | galactosyltransferase | |
| | 1-like | |
| Solanum tuberosum | putative | XM_006363751.2 |
| | glycosyltransferase 7 | |
| Solanum tuberosum | putative | XM_006363750.2 |
| | glycosyltransferase 7 | |
| Medicago truncatula | galactosyl transferase | XM_003611508.2 |
| Medicago truncatula | unidentified | CT573500.2 |
| Solanum lycopersicum | putative | XM_004231889.3 |
| | glycosyltransferase 7 | |
| Solanum lycopersicum | unidentified | AC226502.1 |
| Lycopersicon esculentum | unidentified | BT013963.1 |
| Sesamum indicum | glycosyltransferase | XM_011101097.2 |
| | 6-like | |
| Cucumis melo | galactomannan | XM_008442195.2 |
| | galactosyltransferase | |
| | 1-like | |
| Cucumis melo | unidentified | LN713262.1 |
| Cucumis melo | unidentified | LN681875.1 |
| Solanum lycopersicum | unidentified | HG975514.1 |
| Solanum pennellii | putative | XM_015208067.1 |
| | glycosyltransferase 7 | |
| Solanum pennellii | unidentified | HG975441.1 |
| Sesamum indicum | glycosyltransferase 6 | XM_011075379.2 |
| Coffea arabica | galactomannan | EU568117.1 |
| | galactosyltransferase | |
| Cicer arietinum | galactomannan | XM_012718954.1 |
| | galactosyltransferase | |
| | 1-like | |
| Theobroma cacao | putative | XM_007047507.2 |
| | glycosyltransferase 7 | |
| Theobroma cacao | unidentified | LT594788.1 |
| Erythranthe guttatus | glycosyltransferase | XM_012998642.1 |
| | 6-like | |
| Populus trichocarpa | galactosyltransferase | XM_002310854.2 |
| | family protein | |
| Prunus persica | glycosyltransferase 6 | XM_007208119.2 |
| Nicotiana attenuata | putative | XM_019380314.1 |
| | glycosyltransferase 7 | |
| Gossypium arboreum | putative | XM_017765398.1 |
| | glycosyltransferase 7 | |
| Gossypium arboreum | putative | XM_017765397.1 |
| | glycosyltransferase 7 | |
| Gossypium hirsutum | putative | XM_016879483.1 |
| | glycosyltransferase 7 | |
| Gossypium hirsutum | putative | XM_016879482.1 |
| | glycosyltransferase 7 | |
| Gossypium raimondii | putative | XM_012581704.1 |
| | glycosyltransferase 7 | |
| Gossypium raimondii | putative | XM_012581703.1 |
| | glycosyltransferase 7 | |
| Nicotiana | putative | XM_009626576.2 |
| tomentosiformis | glycosyltransferase 7 | |
| Prunus mume | glycosyltransferase | XM_008239940.2 |
| | 6-like | |
| Populus euphratica | putative | XM_011033007.1 |
| | glycosyltransferase 7 | |
| Populus euphratica | putative | XM_011017963.1 |
| | glycosyltransferase 7 | |
| Musa acuminata subsp. | probable | XM_009396086.2 |
| malaccensis | glycosyltransferase 7 | |
| Daucus carota subsp. | putative | XM_017387245.1 |
| sativus | glycosyltransferase 7 | |
| Daucus carota subsp. | putative | XM_017387244.1 |
| sativus | glycosyltransferase 7 | |
| Gossypium hirsutum | putative | XM_016818092.1 |
| | glycosyltransferase 7 | |
| Gossypium raimondii | putative | XM_012614197.1 |
| | glycosyltransferase 7 | |
| Arabis alpina | unidentified | LT669794.1 |
| Eucalyptus grandis | putative | XM_010029779.2 |
| | glycosyltransferase 7 | |
| Nicotiana tabacum | putative | XM_016593700.1 |
| | glycosyltransferase 7 | |
| Nicotiana sylvestris | putative | XM_009763295.1 |
| | glycosyltransferase 7 | |
| Gossypium hirsutum | putative | XM_016864575.1 |
| | glycosyltransferase 7 | |
| Gossypium arboreum | galactomannan | XM_017774502.1 |
| | galactosyltransferase 1 | |
| Gossypium hirsutum | putative | XM_016850999.1 |
| | glycosyltransferase 7 | |
| Gossypium hirsutum | glycosyltransferase | XM_016841848.1 |
| | 6-like | |
| Gossypium raimondii | glycosyltransferase | XM_012636540.1 |
| | 6-like | |
| Gossypium arboreum | putative | XM_017768866.1 |
| | glycosyltransferase 7 | |
| Raphanus sativus | glycosyltransferase | XM_018611230.1 |
| | 6-like | |
| Jatropha curcas | galactomannan | XM_012232035.2 |
| | galactosyltransferase 1 | |
| Ricinus communis | galactomannan | XM_002513376.2 |
| | galactosyltransferase 1 | |
| Raphanus sativus | glycosyltransferase 6 | XM_018626243.1 |
| Prunus mume | putative | XM_008227859.1 |
| | glycosyltransferase 7 | |

In one aspect, the DNA construct has the following genetic components: a) a gene that expresses zinc-related protein/oxidase, b) a gene that expresses silicatein, c) a gene that expresses silaffin, and d) a gene that expresses alcohol dehydrogenase II. In an alternative aspect, the DNA construct further has e) a gene that expresses lipase.

In another aspect, the DNA construct has the following genetic components: a) a gene that expresses chitin synthase, b) a gene that expresses chitosanase, and c) a gene that expresses chitin deacetylase. In a further aspect, the DNA construct further has d) a gene that expresses lipase.

In still another aspect, the DNA construct has the following genetic components: a) a gene that expresses cellulose synthase and b) a gene that expresses galactomannan galactosyltransferase. In an alternative aspect, the DNA construct further has c) a gene that expresses lipase.

In another aspect, said construct further includes a) a promoter, b) a terminator or stop sequence, c) a gene that confers resistance to an antibiotic (a “selective marker”), d) a reporter protein, or a combination thereof.

In one aspect, the construct includes a regulatory sequence. In a further aspect, the regulatory sequence is already incorporated into a vector such as, for example, a plasmid, prior to genetic manipulation of the vector. In another aspect, the regulatory sequence can be incorporated into the vector through the use of restriction enzymes or any other technique known in the art.

In one aspect, the regulatory sequence is an operon such as, for example, the LAC operon. As used herein, an “operon” is a segment of DNA containing a group of genes wherein the group is controlled by a single promoter. Genes included in an operon are all transcribed together. In a further aspect, the operon is a LAC operon and can be induced when lactose crosses the cell membrane of the biological device.

In one aspect, the regulatory sequence is a promoter. The term “promoter” refers to a DNA sequence capable of controlling the expression of a coding sequence. In another aspect, the coding sequence to be controlled is located 3′ to the promoter. In still another aspect, the promoter is derived from a native gene. In an alternative aspect, the promoter is composed of multiple elements derived from different genes and/or promoters. A promoter can be assembled from elements found in nature, from artificial and/or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, such as, for example, in response to the presence or absence of a particular metabolite (e.g., lactose or tryptophan), a metal ion, a molecule secreted by a pathogen, or the like. A promoter also may be regulated physically, such as, for example, in response to heat, cold, water stress, salt stress, oxygen concentration, illumination, wounding, or the like.

Promoters that are useful to drive expression of the nucleotide sequences described herein are numerous and familiar to those skilled in the art. Suitable promoters include, but are not limited to, the following: T3 promoter, T7 promoter, an iron promoter, and GAL1 promoter. In a further aspect, the promoter is a native part of the vector used herein. Variants of these promoters are also contemplated. The skilled artisan will be able to use site-directed mutagenesis and/or other mutagenesis techniques to modify the promoters to promote more efficient function. The promoter may be positioned, for example, from 10-100 nucleotides from a ribosomal binding site. In another aspect, the promoter is positioned before the gene that expresses zinc-related protein/oxidase, the gene that expresses silicatein, the gene that expresses silaffin, the gene that expresses alcohol dehydrogenase II, the gene that expresses lipase, the gene that expresses chitin synthase, the gene that expresses chitosanase, the gene that expresses chitin deacetylase, the gene that expresses cellulose synthase, the gene that expresses galactomannan galactosyltransferase, or any combination thereof.

In one aspect, the promoter is a GAL1 promoter. In another aspect, the GAL1 promoter is native to the plasmid used to create the vector. In another aspect, a GAL1 promoter is positioned before the gene that expresses zinc-related protein/oxidase, the gene that expresses silicatein, the gene that expresses silaffin, the gene that expresses alcohol dehydrogenase II, or any combination thereof. In another aspect, the promoter is a GAL1 promoter obtained from or native to the pYES2 plasmid.

In another aspect, the promoter is a T7 promoter. In a further aspect, the T7 promoter is native to the plasmid used to create the vector. In still another aspect, the T7 promoter is positioned before any or all of the genes in the construct, or is positioned before the LAC operon. In yet another aspect, the promoter is a T7 promoter obtained from or native to the pETDuet-1 plasmid. In one aspect, the promoter has SEQ ID NO. 13 or a derivative or variant thereof.

In another aspect, the regulatory sequence is a terminator or stop sequence. As used herein, a terminator is a sequence of DNA that marks the end of a gene or operon to be transcribed. In a further aspect, the terminator is an intrinsic terminator or a Rho-dependent transcription terminator. As used herein, an intrinsic terminator is a sequence wherein a hairpin structure can form in the nascent transcript that disrupts the mRNA/DNA/RNA polymerase complex. In one aspect, the terminator is a T7 terminator. In an alternative aspect, the terminator is a CYC1 terminator obtained from or native to the pYES2 plasmid.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2000 nucleotides in the 5′ direction from the start codon of the DNA to be transcribed, or may be located 3′ to the DNA to be transcribed. Enhancers may be “cis-acting, that is, located on the same molecule of DNA whose expression they affect.

In certain aspects, the DNA construct includes a ribosomal binding site. In one aspect, the ribosomal binding site in the DNA construct is AGGAGG or a derivative or variant thereof. In one aspect, the ribosomal binding site is native to the vector used herein. In certain aspects, when the DNA construct further includes a ribosomal switch. In one aspect, the ribosomal switch has SEQ ID NO. 14 or at least 70% homology thereto. In some aspects, the ribosomal binding site and optional ribosomal switch are positioned after the gene for galactomannan galactosyltransferase from 5′ to 3′.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for restriction enzymes. Further, restriction sites may be added, disabled, or removed as required, using techniques known in the art. In one aspect, the plasmid contains restriction sites for any known restriction enzyme such as, for example, HindIII, KpnI, SacI, BamHI, BstXI, EcoRI, BasBI, NotI, XhoI, XphI, XbaI, ApaI, SalI, ClaI, EcoRV, PstI, SmaI, XmaI, SpeI, EagI, SacII, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA at a distance of from 20 bases to over 1000 bases away from the restriction site. A variety of restriction enzymes are commercially available and their recognition sequences, as well as instructions for use (e.g., amount of DNA needed, precise volumes of reagents, purification techniques, as well as information about salt concentration, pH, optimum temperature, incubation time, and the like) are provided by enzyme manufacturers.

In one aspect, a plasmid with a polylinker containing one or more restriction sites can be digested with one restriction enzyme and a nucleotide sequence of interest can be ligated into the plasmid using a commercially-available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, usually starting just after a promoter, the order and direction of elements inserted into a plasmid can be especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleotide fragments into the plasmid.

In one aspect, different genes can be ligated into a plasmid in one pot. In this aspect, the genes will first be digested with restriction enzymes. In certain aspects, the digestion of genes with restriction enzymes provides multiple pairs of matching 5′ and 3′ overhangs that will spontaneously assemble the genes in the desired order. In another aspect, the genes and components to be incorporated into a plasmid can be assembled into a single insert sequence prior to insertion into the plasmid. In a further aspect, a DNA ligase enzyme can be used to assist in the ligation process.

In another aspect, the ligation mix may be incubated in an electromagnetic chamber. In one aspect, the incubation lasts for about 1 minute, about 2 minutes, about 5 minutes, about 10 minutes, about 15 minutes, about 20 minutes, about 30 minutes, or about 1 hour.

The DNA construct described herein can be part of a vector. In general, plasmid vectors containing replicon and control sequences that are derived from species compatible with the host cell are used in connection with the hosts. The vector ordinarily carries a replication site as well as marking sequences that are capable of performing phenotypic selection in transformed cells. Plasmid vectors are well known and commercially available. Such vectors include, but are not limited to, pWLneo, pSV2cat, pOG44, pXT1, pSG, pSVK3, pBSK, pYES, pYES2, pBSKII, pUC, pUC19, and pETDuet-1 vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copies per host cell and will contain selectable markers (such as genes for antibiotic resistance) to allow the skilled artisan to select host cells that have been successfully transfected with the plasmids (for example, by growing the host cells in a medium containing the antibiotic).

In one aspect, the vector encodes a selection marker. In a further aspect, the selection marker is a gene that confers resistance to an antibiotic. In certain aspects, during fermentation of host cells transformed with the vector, the cells are contacted with the antibiotic. For example, the antibiotic may be included in the culture medium. Cells that have not been successfully transformed cannot survive in the presence of the antibiotic; only cells containing the vector, which confers antibiotic resistance, can survive. Optimally, only cells containing the vector to be expressed will be cultured, as this will result in the highest production efficiency of the desired gene products (e.g., peptides). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isoniazid, methicillin, oxacillin, vancomycin, streptomycin, quinolones, rifampin, rifampicin, sulfonamides, cephalothin, erythromycin, gentamycin, penicillin, other commonly-used antibiotics, or a combination thereof.

In certain aspects, the DNA construct can include a gene that expresses a reporter protein. The selection of the reporter protein can vary. For example, the reporter protein can be a yellow fluorescent protein, a red fluorescent protein, a green fluorescent protein, or a cyan fluorescent protein. In one aspect, the reporter protein is a yellow fluorescent protein and the gene that expresses the reporter protein has SEQ ID NO. 15 or at least 70% homology thereto. The amount of fluorescence that is produced can be correlated to the amount of DNA incorporated into the transfected cells. The fluorescence produced can be detected and quantified using techniques known in the art. For example, spectrofluorometers are typically used to measure fluorescence.

The DNA construct described herein can be part of a vector. In one aspect, the vector is a plasmid, a phagemid, a cosmid, a yeast artificial chromosome, a bacterial artificial chromosome, a virus, a phage, or a transposon.

Exemplary methods for producing the DNA constructs described herein are provided in the Examples. Restriction enzymes and purification techniques known in the art can be used to assemble the DNA constructs. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the methods described below.

In one aspect, the construct includes from 5′ to 3′ the following genetic components in the following order: (1) a gene that expresses zinc-related protein/oxidase, (2) a gene that expresses silicatein, (3) a gene that expresses silaffin, and (4) a gene that expresses alcohol dehydrogenase II.

In another aspect, the construct comprises from 5′ to 3′ the following genetic components in the following order: (1) a gene that expresses zinc-related protein/oxidase, (2) a CYC1 terminator, (3) a GAL1 promoter, (4) a gene that expresses silicatein, (5) a CYC1 terminator, (6) a GAL1 promoter, (7) a gene that expresses silaffin, (8) a CYC1 terminator, (9) a GAL1 promoter, (10) a gene that expresses alcohol dehydrogenase II, and (11) a CYC1 terminator.

In another aspect, the construct includes from 5′ to 3′ the following genetic components in the following order: a gene that expresses zinc-related protein/oxidase having SEQ ID NO. 1 or at least 70% homology thereto, a gene that expresses silicatein having SEQ ID NO. 2 or at least 70% homology thereto, a gene that expresses silaffin having SEQ ID NO. 3 or at least 70% homology thereto, and a gene that expresses alcohol dehydrogenase II having SEQ ID NO. 4 or at least 70% homology thereto.

In one aspect, the construct includes from 5′ to 3′ the following genetic components in the following order: a gene that expresses zinc-related protein/oxidase having SEQ ID NO. 1 or at least 90% homology thereto, a gene that expresses silicatein having SEQ ID NO. 2 or at least 90% homology thereto, a gene that expresses silaffin having SEQ ID NO. 3 or at least 90% homology thereto, and a gene that expresses alcohol dehydrogenase II having SEQ ID NO. 4 or at least 90% homology thereto.

In another aspect, the construct includes from 5′ to 3′ the following genetic components in the following order: a) a gene that expresses zinc-related protein/oxidase having SEQ ID NO. 1 or at least 90% homology thereto, b) a CYC1 terminator, c) a GAL1 promoter, d) a gene that expresses silicatein having SEQ ID NO. 2 or at least 90% homology thereto, e) a CYC1 terminator, f) a GAL1 promoter, g) a gene that expresses silaffin having SEQ ID NO. 3 or at least 90% homology thereto, h) a CYC1 terminator, i) a GAL1 promoter, a gene that expresses alcohol dehydrogenase II having SEQ ID NO. 4 or at least 90% homology thereto, and j) a CYC1 terminator.

In another aspect, the construct is a pYES2 plasmid having from 5′ to 3′ the following genetic components in the following order: a) a gene that expresses zinc-related protein/oxidase having SEQ ID NO. 1 or at least 70% homology thereto, b) a CYC1 terminator, c) a GAL1 promoter, d) a gene that expresses silicatein having SEQ ID NO. 2 or at least 70% homology thereto, e) a CYC1 terminator, f) a GAL1 promoter, g) a gene that expresses silaffin having SEQ ID NO. 3 or at least 70% homology thereto, h) a CYC1 terminator, i) a GAL1 promoter, and j) a gene that expresses alcohol dehydrogenase II having SEQ ID NO. 4 or at least 70% homology thereto, k) and a CYC1 terminator.

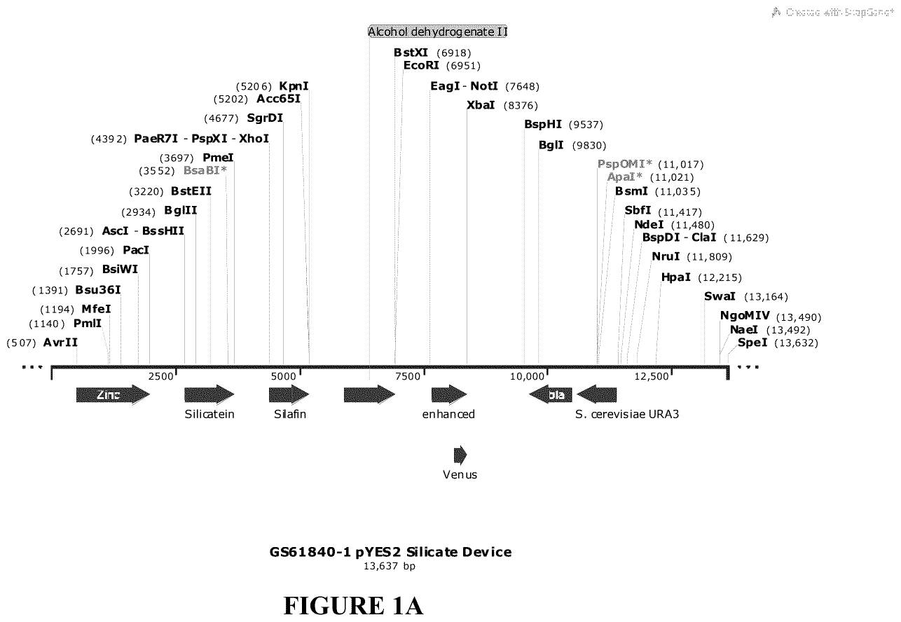

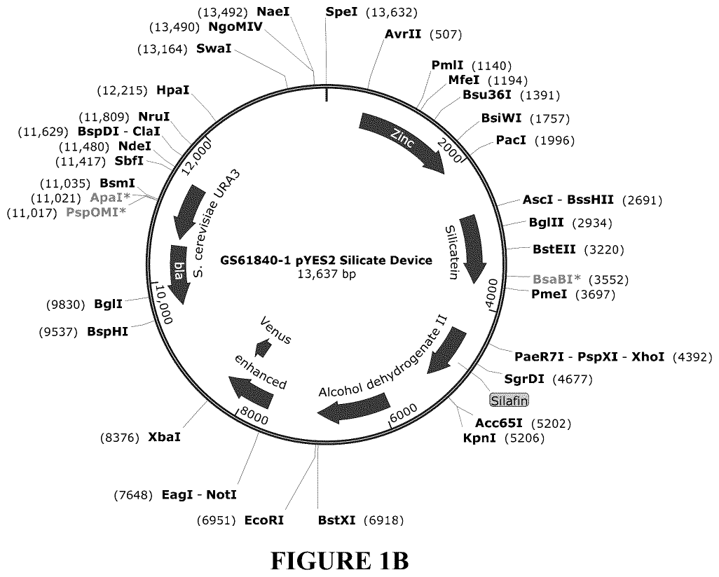

In another aspect, the DNA construct is SEQ ID NO. 16 or has at least 90% homology thereto. In a further aspect, the DNA construct is the vector depicted in FIGS. 1A and 1B.

In one aspect, the construct includes from 5′ to 3′ the following genetic components in the following order: (1) a gene that expresses lipase, (2) a gene that expresses zinc-related protein/oxidase, (3) a gene that expresses silicatein, (4) a gene that expresses silaffin, and (5) a gene that expresses alcohol dehydrogenase II.

In another aspect, the construct includes from 5′ to 3′ the following genetic components in the following order: (1) a gene that expresses lipase, (2) a CYC1 terminator, (3) a GAL1 promoter, (4) a gene that expresses zinc-related protein/oxidase, (5) a CYC1 terminator, (6) a GAL1 promoter, (7) a gene that expresses silicatein, (8) a CYC1 terminator, (9) a GAL1 promoter, (10) a gene that expresses silaffin, (11) a CYC1 terminator, (12) a GAL1 promoter, (13) a gene that expresses alcohol dehydrogenase II, and (14) a CYC1 terminator.

In one aspect, the construct includes from 5′ to 3′ the following genetic components in the following order: (1) a gene that expresses lipase having SEQ ID NO. 5 or at least 70% homology thereto, (2) a gene that expresses zinc-related protein/oxidase having SEQ ID NO. 1 or at least 70% homology thereto, (3) a gene that expresses silicatein having SEQ ID NO. 2 or at least 70% homology thereto, (4) a gene that expresses silaffin having SEQ ID NO. 3 or at least 70% homology thereto, and (5) a gene that expresses alcohol dehydrogenase II having SEQ ID NO. 4 or at least 70% homology thereto.

In one aspect, the construct includes from 5′ to 3′ the following genetic components in the following order: (1) a gene that expresses lipase having SEQ ID NO. 5 or at least 90% homology thereto, (2) a gene that expresses zinc-related protein/oxidase having SEQ ID NO. 1 or at least 90% homology thereto, (3) a gene that expresses silicatein having SEQ ID NO. 2 or at least 90% homology thereto, (4) a gene that expresses silaffin having SEQ ID NO. 3 or at least 90% homology thereto, and (5) a gene that expresses alcohol dehydrogenase II having SEQ ID NO. 4 or at least 90% homology thereto.

In another aspect, the construct is a pYES2 plasmid having from 5′ to 3′ the following genetic components in the following order: (a) a gene that expresses lipase having SEQ ID NO. 5 or at least 70% homology thereto, (b) a CYC1 terminator, (c) a gene that expresses zinc-related protein/oxidase having SEQ ID NO. 1 or at least 70% homology thereto, (d) a CYC1 terminator, (e) a GAL1 promoter, (f) a gene that expresses silicatein having SEQ ID NO. 2 or at least 70% homology thereto, (g) a CYC1 terminator, (h) a GAL1 promoter, (i) a gene that expresses silaffin having SEQ ID NO. 3 or at least 70% homology thereto, (j) a CYC1 terminator, (k) a GAL1 promoter, (1) a gene that expresses alcohol dehydrogenase II having SEQ ID NO. 4 or at least 70% homology thereto, and (m) a CYC1 terminator.

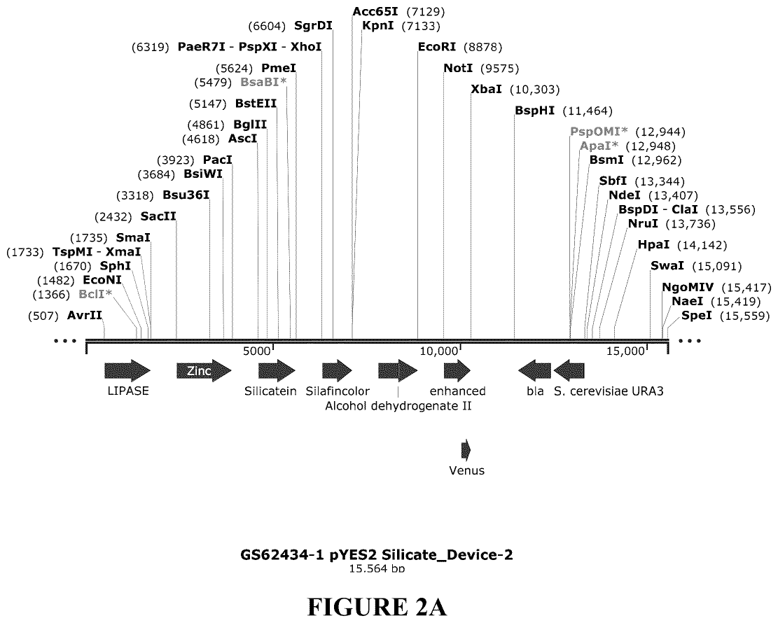

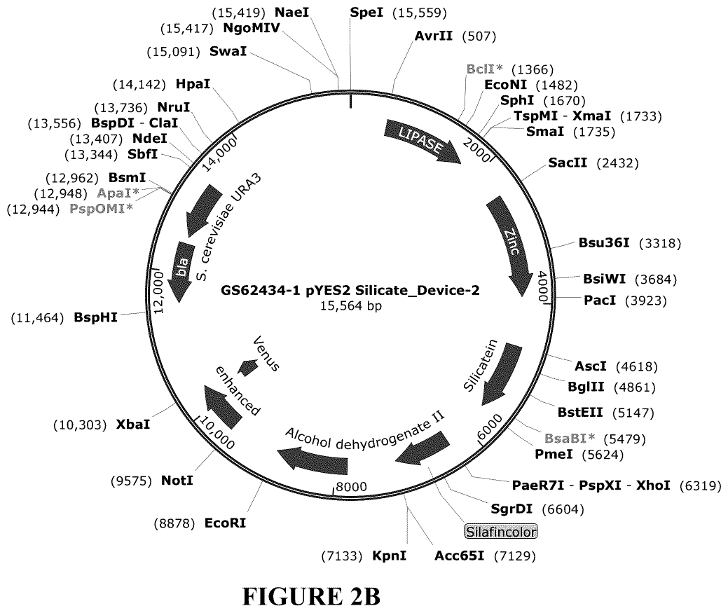

In another aspect, the DNA construct is SEQ ID NO. 17 or has at least 90% homology thereto. In a further aspect, the DNA construct is the vector depicted in FIGS. 2A and 2B.

In one aspect, the construct for producing the polyactive carbohydrate includes from 5′ to 3′ the following genetic components in the following order: (1) a gene that expresses chitin synthase, (2) a gene that expresses chitosanase, and (3) a gene that expresses chitin deacetylase.

In another aspect, the construct for producing the polyactive carbohydrate includes from 5′ to 3′ the following genetic components in the following order: (1) a gene that expresses chitin synthase, (2) a CYC1 terminator, (3) a GAL1 promoter, (4) a gene that expresses chitosanase, (5) a CYC1 terminator, (6) a GAL1 promoter, (7) a gene that expresses chitin deacetylase, and (8) a CYC1 terminator.

In another aspect, the construct for producing the polyactive carbohydrate includes from 5′ to 3′ the following genetic components in the following order: (1) a gene that expresses chitin synthase having SEQ ID NO. 6 or at least 70% homology thereto, (2) a gene that expresses chitosanase having SEQ ID NO. 7 or at least 70% homology thereto, and (3) a gene that expresses chitin deacetylase having SEQ ID NO. 8 or at least 70% homology thereto.

In another aspect, the construct for producing the polyactive carbohydrate includes from 5′ to 3′ the following genetic components in the following order: (1) a gene that expresses chitin synthase having SEQ ID NO. 6 or at least 90% homology thereto, (2) a gene that expresses chitosanase having SEQ ID NO. 7 or at least 90% homology thereto, and (3) a gene that expresses chitin deacetylase having SEQ ID NO. 8 or at least 90% homology thereto.

In another aspect, the construct for producing the polyactive carbohydrate is a pYES2 plasmid having from 5′ to 3′ the following genetic components in the following order: (a) a gene that expresses chitin synthase having SEQ ID NO. 6 or at least 70% homology thereto, (b) a CYC1 terminator, (c) a GAL1 promoter, (d) a gene that expresses chitosanase having SEQ ID NO. 7 or at least 70% homology thereto, (e) a CYC1 terminator, (f) a GAL1 promoter, (g) a gene that expresses chitin deacetylase having SEQ ID NO. 8 or at least 70% homology thereto, and (h) a CYC1 terminator.

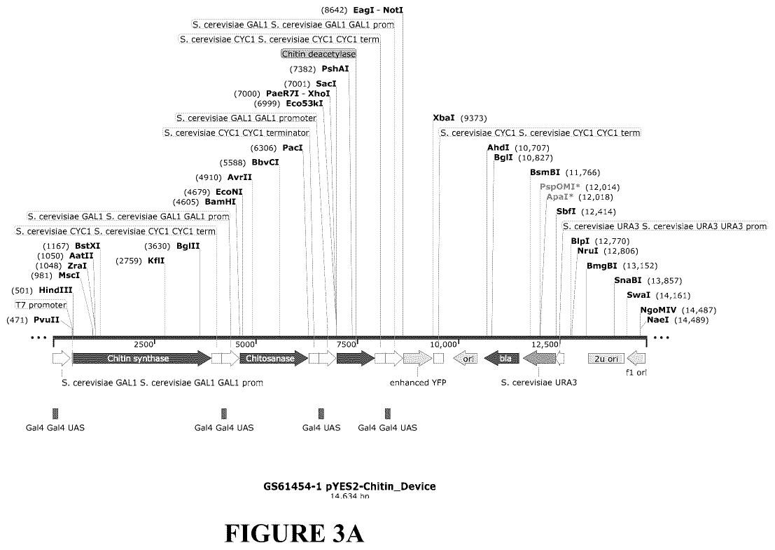

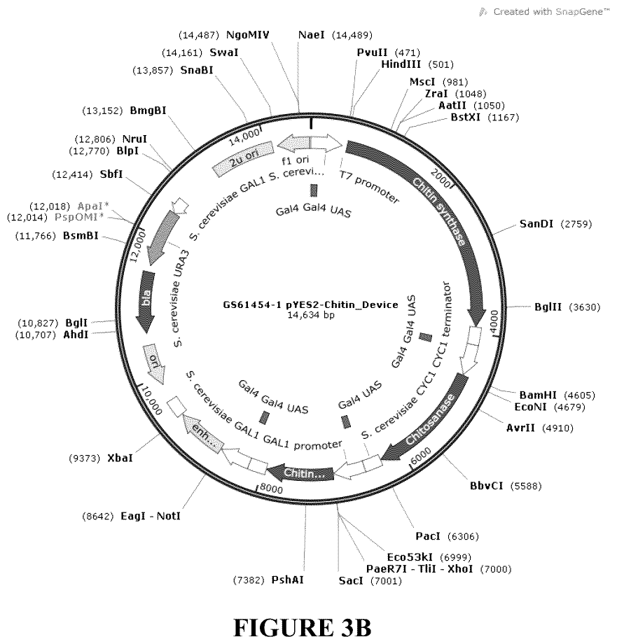

In another aspect, the DNA construct for producing the polyactive carbohydrate is SEQ ID NO. 18 or has at least 90% homology thereto. In a further aspect, the DNA construct is the vector depicted in FIGS. 3A and 3B.

In one aspect, the construct for producing the polyactive carbohydrate includes from 5′ to 3′ the following genetic components in the following order: (1) a gene that expresses lipase, (2) a gene that expresses chitin synthase, (3) a gene that expresses chitosanase, and (4) a gene that expresses chitin deacetylase.

In another aspect, the construct for producing the polyactive carbohydrate includes from 5′ to 3′ the following genetic components in the following order: (1) a gene that expresses lipase, (2) a CYC1 terminator, (3) a GAL1 promoter, (4) a gene that expresses chitin synthase, (5) a CYC1 terminator, (6) a GAL1 promoter, (7) a gene that expresses chitosanase, (8) a CYC1 terminator, (9) a GAL1 promoter, (10) a gene that expresses chitin deacetylase, and (11) a CYC1 terminator.

In another aspect, the construct for producing the polyactive carbohydrate includes from 5′ to 3′ the following genetic components in the following order: (1) a gene that expresses lipase having SEQ ID NO. 5 or at least 70% homology thereto, (2) a gene that expresses chitin synthase having SEQ ID NO. 6 or at least 70% homology thereto, (3) a gene that expresses chitosanase having SEQ ID NO. 7 or at least 70% homology thereto, and (4) a gene that expresses chitin deacetylase having SEQ ID NO. 8 or at least 70% homology thereto.

In another aspect, the construct for producing the polyactive carbohydrate includes from 5′ to 3′ the following genetic components in the following order: (1) a gene that expresses lipase having SEQ ID NO. 5 or at least 90% homology thereto, (2) a gene that expresses chitin synthase having SEQ ID NO. 6 or at least 90% homology thereto, (3) a gene that expresses chitosanase having SEQ ID NO. 7 or at least 90% homology thereto, and (4) a gene that expresses chitin deacetylase having SEQ ID NO. 8 or at least 90% homology thereto.

In another aspect, the construct for producing the polyactive carbohydrate is a pYES2 plasmid having from 5′ to 3′ the following genetic components in the following order: (a) a gene that expresses lipase having SEQ ID NO. 5 or at least 70% homology thereto, (b) a CYC1 terminator, (c) a GAL1 promoter, (d) a gene that expresses chitin synthase having SEQ ID NO. 6 or at least 70% homology thereto, (e) a CYC1 terminator, (f) a GAL1 promoter, (g) a gene that expresses chitosanase having SEQ ID NO. 7 or at least 70% homology thereto, (h) a CYC1 terminator, (i) a GAL1 promoter, (j) a gene that expresses chitin deacetylase having SEQ ID NO. 8 or at least 70% homology thereto, and (k) a CYC1 terminator.

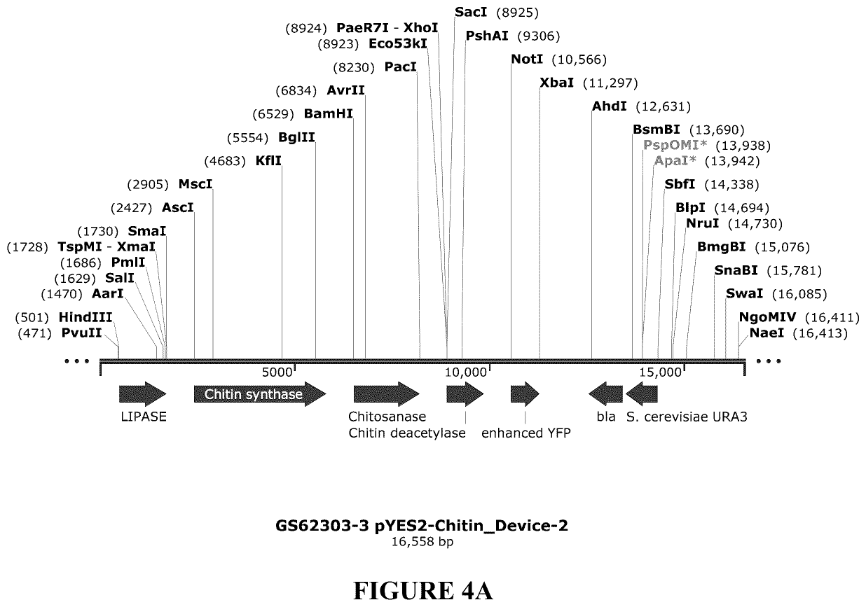

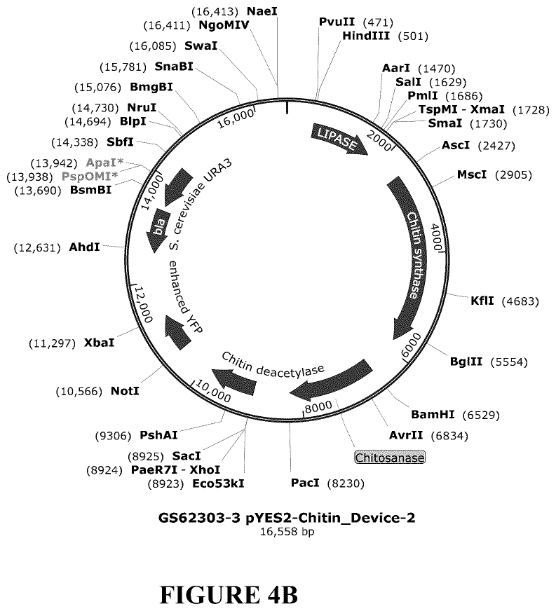

In another aspect, the DNA construct for producing the polyactive carbohydrate is SEQ ID NO. 19 or has at least 90% homology thereto. In a further aspect, the DNA construct is the vector depicted in FIGS. 4A and 4B.

In one aspect, the construct for producing the carbo sugar includes from 5′ to 3′ the following genetic components in the following order: (1) a gene that expresses cellulose synthase and (2) a gene that expresses galactomannan galactosyltransferase.

In another aspect, the construct for producing the carbo sugar includes from 5′ to 3′ the following genetic components in the following order: (1) a gene that expresses cellulose synthase, (2) a CYC1 terminator, (3) a GAL1 promoter, (4) a gene that expresses galactomannan galactosyltransferase, and (5) a CYC1 terminator.

In one aspect, the construct for producing the carbo sugar includes from 5′ to 3′ the following genetic components in the following order: (1) a gene that expresses cellulose synthase having SEQ ID NO. 9 or at least 70% homology thereto and (2) a gene that expresses galactomannan galactosyltransferase having SEQ ID NO. 10 or at least 70% homology thereto.

In one aspect, the construct for producing the carbo sugar includes from 5′ to 3′ the following genetic components in the following order: (1) a gene that expresses cellulose synthase having SEQ ID NO. 9 or at least 90% homology thereto and (2) a gene that expresses galactomannan galactosyltransferase having SEQ ID NO. 10 or at least 90% homology thereto.

In another aspect, the construct for producing the carbo sugar is a pYES2 plasmid having from 5′ to 3′ the following genetic components in the following order: (a) a gene that expresses cellulose synthase having SEQ ID NO. 9 or at least 70% homology thereto, (b) a CYC1 terminator, (c) a GAL1 promoter, (d) a gene that expresses galactomannan galactosyltransferase having SEQ ID NO. 10 or at least 70% homology thereto, and (5) a CYC1 terminator.

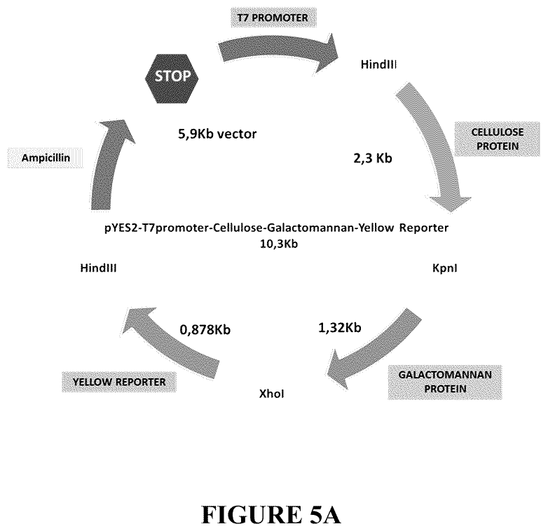

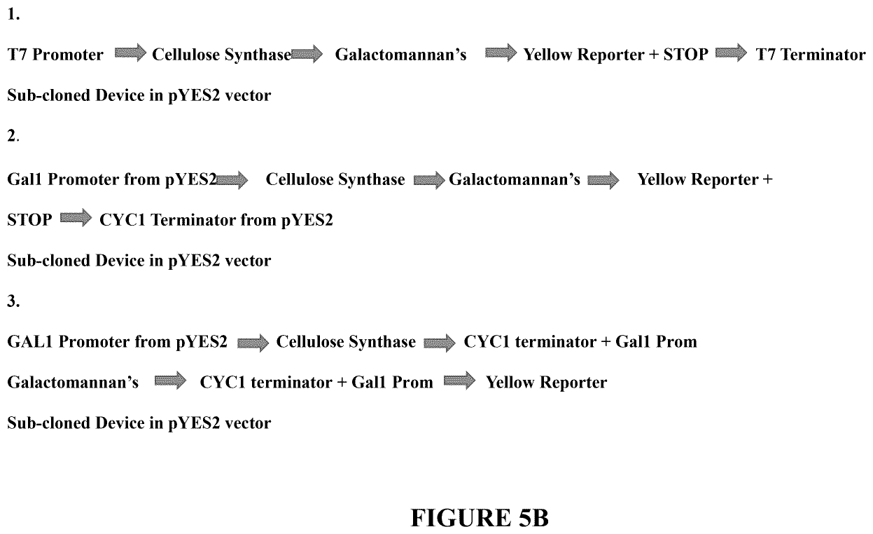

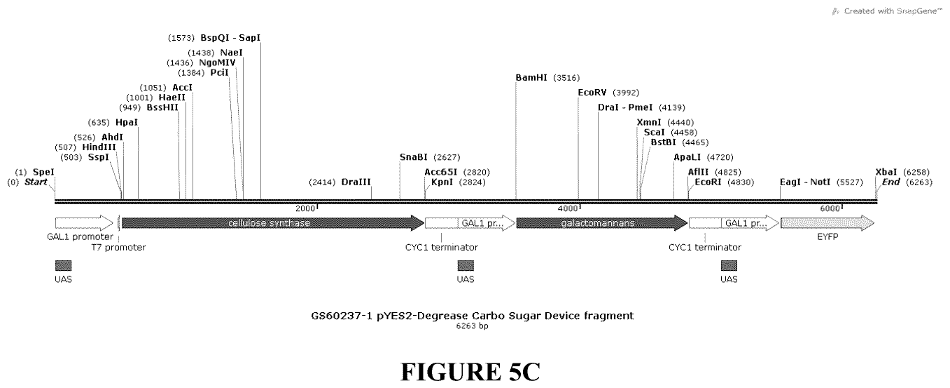

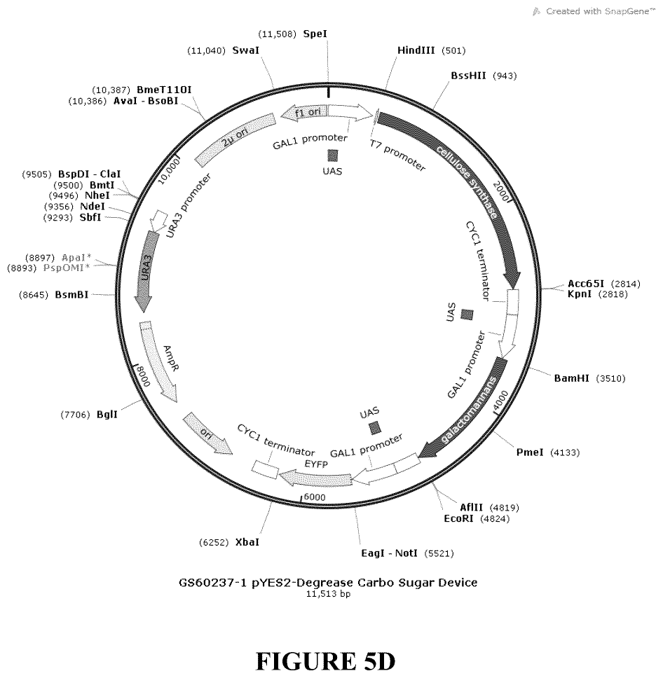

In another aspect, the DNA construct for producing the carbo sugar is SEQ ID NO. 20 or has at least 90% homology thereto. In a further aspect, the DNA construct is the vector depicted in FIGS. 5A-5D.

In one aspect, the construct for producing the carbo sugar includes from 5′ to 3′ the following genetic components in the following order: (1) a gene that expresses lipase, (2) a gene that expresses cellulose synthase and (3) a gene that expresses galactomannan galactosyltransferase.

In another aspect, the construct for producing the carbo sugar includes from 5′ to 3′ the following genetic components in the following order: (1) a gene that expresses lipase, (2) a CYC1 terminator, (3) a GAL1 promoter, (4) a gene that expresses cellulose synthase, (5) a CYC1 terminator, (6) a GAL1 promoter, (7) a gene that expresses galactomannan galactosyltransferase, and (8) a CYC1 terminator.

In one aspect, the construct for producing the carbo sugar includes from 5′ to 3′ the following genetic components in the following order: (1) a gene that expresses lipase having SEQ ID NO. 5 or at least 70% homology thereto, (2) a gene that expresses cellulose synthase having SEQ ID NO. 9 or at least 70% homology thereto, and (3) a gene that expresses galactomannan galactosyltransferase having SEQ ID NO. 10 or at least 70% homology thereto.

In one aspect, the construct for producing the carbo sugar includes from 5′ to 3′ the following genetic components in the following order: (1) a gene that expresses lipase having SEQ ID NO. 5 or at least 90% homology thereto, (2) a gene that expresses cellulose synthase having SEQ ID NO. 9 or at least 90% homology thereto, and (3) a gene that expresses galactomannan galactosyltransferase having SEQ ID NO. 10 or at least 90% homology thereto.

In another aspect, the construct for producing the carbo sugar is a pYES2 plasmid having from 5′ to 3′ the following genetic components in the following order: (a) a gene that expresses cellulose synthase having SEQ ID NO. 9 or at least 70% homology thereto, (b) a CYC1 terminator, (c) a GAL1 promoter, (d) a gene that expresses galactomannan galactosyltransferase having SEQ ID NO. 10 or at least 70% homology thereto, and (5) a CYC1 terminator.

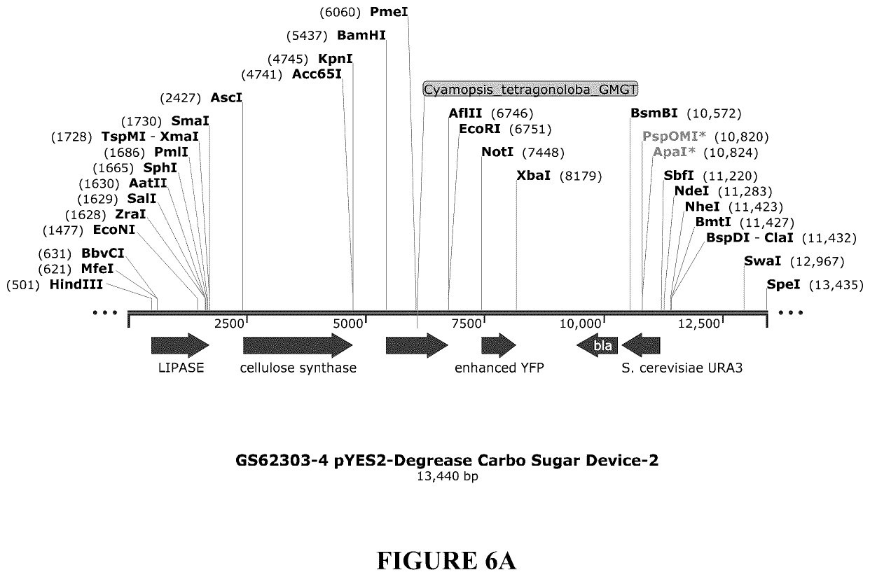

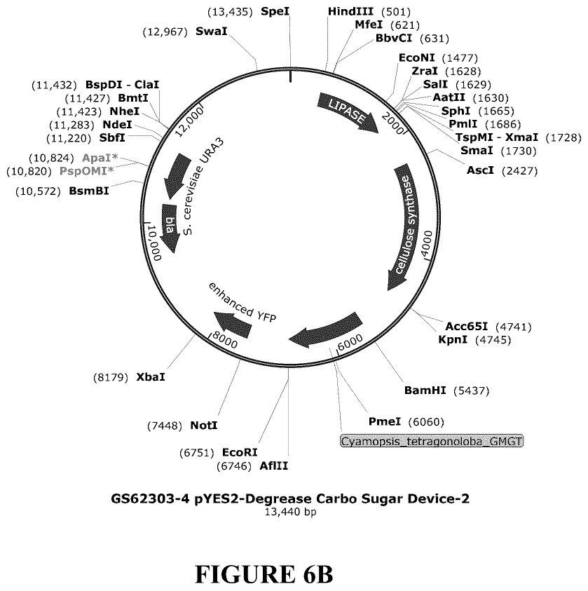

In another aspect, the DNA construct for producing the carbo sugar is SEQ ID NO. 21 or has at least 90% homology thereto. In a further aspect, the DNA construct is the vector depicted in FIGS. 6A-6B.

In one aspect, the construct for producing the carbo sugar includes from 5′ to 3′ the following genetic components in the following order: (1) a gene that expresses lipase, (2) a gene that expresses cellulose synthase, and (3) a gene that expresses galactomannan galactosyltransferase.

In another aspect, the construct for producing the carbo sugar includes from 5′ to 3′ the following genetic components in the following order: (1) a gene that expresses lipase, (2) a T7 promoter, (3) a LAC operon, (4) a riboswitch, (5) a gene that expresses cellulose synthase, (6) a riboswitch, and (7) a gene that expresses galactomannan galactosyltransferase.

In one aspect, the construct for producing the carbo sugar includes from 5′ to 3′ the following genetic components in the following order: (1) a gene that expresses lipase having SEQ ID NO. 5 or at least 70% homology thereto, (2) a gene that expresses cellulose synthase having SEQ ID NO. 9 or at least 70% homology thereto, and (3) a gene that expresses galactomannan galactosyltransferase having SEQ ID NO. 10 or at least 70% homology thereto.

In one aspect, the construct for producing the carbo sugar includes from 5′ to 3′ the following genetic components in the following order: (1) a gene that expresses lipase having SEQ ID NO. 5 or at least 90% homology thereto, (2) a gene that expresses cellulose synthase having SEQ ID NO. 9 or at least 90% homology thereto, and (3) a gene that expresses galactomannan galactosyltransferase having SEQ ID NO. 10 or at least 90% homology thereto.

In another aspect, the construct for producing the carbo sugar is a pETDuet-1 plasmid having from 5′ to 3′ the following genetic components in the following order: (a) a gene that expresses lipase having SEQ ID NO. 5 or at least 70% homology thereto, (b) a T7 promoter, (c) a LAC operon, (d) a riboswitch having SEQ ID NO. 14 or at least 70% homology thereto, (e) a gene that expresses cellulose synthase having SEQ ID NO. 9 or at least 70% homology thereto, (f) a riboswitch having SEQ ID NO. 14 or at least 70% homology thereto, and (e) a gene that expresses galactomannan galactosyltransferase having SEQ ID NO. 10 or at least 70% homology thereto.

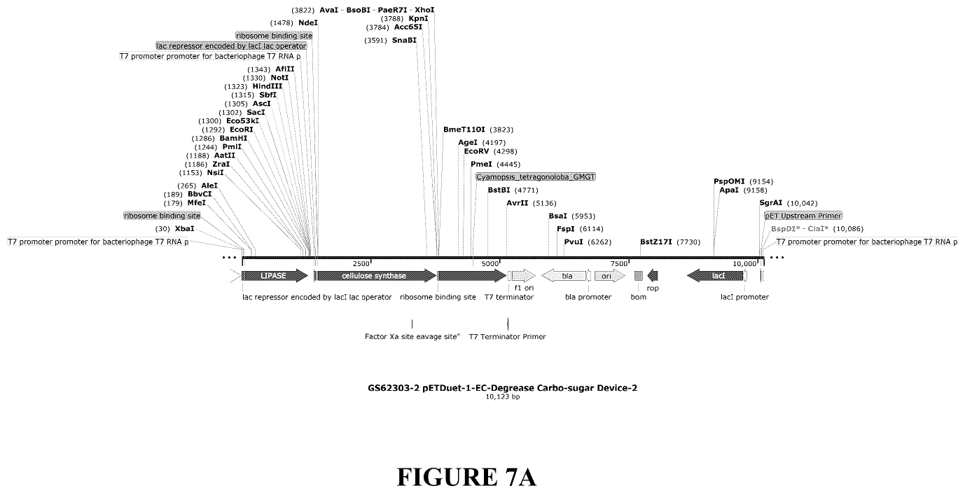

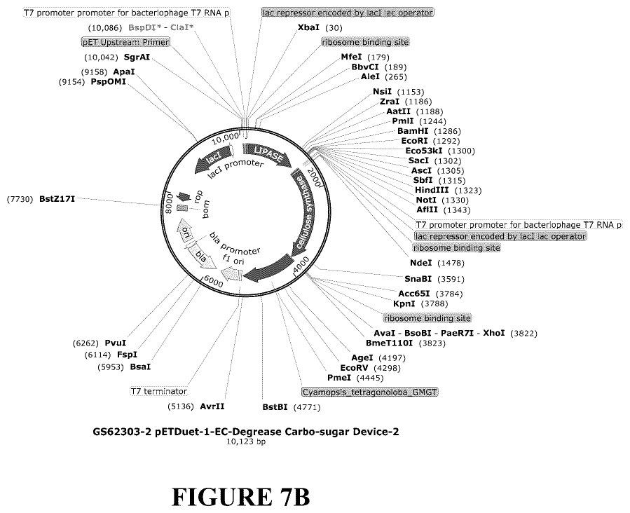

In another aspect, the DNA construct for producing the carbo sugar is SEQ ID NO. 22 or has at least 90% homology thereto. In a further aspect, the DNA construct is the vector depicted in FIGS. 7A-7B.

### II. Biological Devices

In one aspect, a “biological device” is formed when a microbial cell is transfected with the DNA construct described herein. The biological devices are generally composed of microbial host cells, where the host cells are transformed with a DNA construct described herein.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce an anti-microbial and UV-protective extract. “Heterologous” genes and proteins are genes and proteins that have been experimentally inserted into a cell that are not normally expressed by the cell. A heterologous gene may be cloned or derived from a different cell type or species than the recipient cell or organism. Heterologous genes may be introduced into cells by transduction or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes).

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the type of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed only for a limited period of time).

“Competent cells” refers to microbial cells capable of taking up heterologous DNA. Competent cells can be purchased from a commercial source, or cells can be made competent using procedures known in the art. Exemplary procedures for producing competent cells are provided in the examples.

The host cells as referred to herein include their progeny, which are any and all subsequent generations formed by cell division. It is understood that not all progeny may be identical due to deliberate or inadvertent mutations. A host cell may be “transfected” or “transformed,” which refers to a process by which an exogenous nucleic acid is transferred or introduced into the host cell.

A transformed cell includes the primary subject cell and its progeny. The host cells can be naturally-occurring cells or “recombinant” cells. Recombinant cells are distinguishable from naturally-occurring cells in that naturally-occurring cells do not contain heterologous DNA introduced through molecular cloning procedures. In one aspect, the host cell is a bacteria such as, for example, Escherichia coli. In other aspects, the host cell is a eukaryotic cell such as, for example, the yeast Saccharomyces cerevisiae. Host cells transformed with the DNA construct described herein are referred to as “biological devices.”

The DNA construct is first delivered into the host cell. In one aspect, the host cells are naturally competent (i.e., able to take up exogenous DNA from the surrounding environment). In another aspect, cells must be treated to induce artificial competence. This delivery may be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the membranes of the cells through which the vector containing the DNA construct enters. Another method involves exposing intact yeast cells to alkali cations such as, for example, lithium. In one aspect, this method includes exposing the yeast to lithium acetate, polyethylene glycol, and single-stranded DNA such as, for example, salmon sperm DNA. Without wishing to be bound by theory, the single-stranded DNA is thought to bind to the cell wall of the yeast, thereby blocking plasmids from binding. The plasmids are then free to enter the yeast cell. Enzymatic and/or electromagnetic techniques can be used alone, or in combination with other methods, to transform microbial cells. Exemplary procedures for transforming yeast and bacteria with specific DNA constructs are provided in the Examples. In certain aspects, two or more types of DNA can be incorporated into the host cells. Thus, different metabolites can be produced from the same host cells at enhanced rates.

### III. Preparation of Devices and Extracts

The biological devices described herein are useful in the production of anti-microbial and UV-protective extracts as well as in the production of adhesives and hard and soft biofoams. Once the DNA construct has been incorporated into the host cell, the cells are cultured such that the cells multiply. A satisfactory microbiological culture contains available sources of hydrogen donors and acceptors, carbon, nitrogen, sulfur, phosphorus, inorganic salts, and, in certain cases, vitamins or other growth-promoting substances. For example, the addition of peptone provides a readily-available source of nitrogen and carbon. Furthermore, the use of different types of media results in different growth rates and different stationary phase densities; stationary phase is where secondary metabolite production occurs most frequently. A rich media results in a short doubling time and higher cell density at stationary phase. Minimal media results in slow growth and low final cell densities. Efficient agitation and aeration increase final cell densities.

In one aspect, host cells can be cultured or fermented by any method known in the art. The skilled practitioner will be able to select a culture medium based on the species and/or strain of host cell selected. In certain aspects, the culture medium will contain a carbon source. A variety of carbon sources are contemplated, including, but not limited to: monosaccharides such as glucose and fructose, disaccharides such as lactose or sucrose, oligosaccharides, polysaccharides such as starch, or mixtures thereof. Unpurified mixtures extracted from feedstocks are also contemplated and include molasses, barley malt, and related compounds and compositions. Other glycolytic and tricarboxylic acid cycle intermediates are also contemplated as carbon sources, as are one-carbon substrates such as carbon dioxide and/or methanol in the cases of compatible organisms. The carbon source utilized is limited only by the particular organism being cultured.

Culturing or fermenting of host cells can be accomplished by any technique known in the art. In one aspect, batch fermentation can be conducted. In batch fermentation, the composition of the culture medium is set at the beginning and the system is closed to future alterations. In some aspects, a limited form of batch fermentation may be carried out, wherein factors such as oxygen concentration and pH are manipulated, but additional carbon is not added. Continuous fermentation methods are also contemplated. In continuous fermentation, equal amounts of a defined medium are continuously added to and removed from a bioreactor. In other aspects, microbial host cells are immobilized on a substrate. Fermentation may be carried out on any scale and may include methods in which literal “fermentation” is carried out as well as other culture methods that are non-fermentative.

In one aspect, the method involves growing the biological devices described herein for a sufficient time to produce anti-microbial and UV-protective extracts. The ordinary artisan will be able to choose a culture medium and optimum culture conditions based on the biological identity of the host cells.

In one aspect, the UV protective extract can be prepared by exposing a culture of a biological device such as those disclosed herein to UV radiation, then extracting components from the culture. In one aspect, the components are extracted via centrifugation. The UV radiation can be of any wavelength, but in one aspect, it can be shortwave radiation (i.e., ultraviolet C having a wavelength of approximately 100 to 280 nm), medium wave radiation (i.e., ultraviolet B, having a wavelength of approximately 280 to 315 nm), or longwave radiation (i.e., ultraviolet A having a wavelength of 315 to 400 nm). In one aspect, the culture of the biological device can be irradiated with a 254 nm shortwave UV source. In another aspect, the culture of the biological device can be irradiated with a 365 nm longwave UV source. In still another aspect, the culture of the biological device can be irradiated with both a 254 nm and a 365 nm UV source. In yet another aspect, the culture of the biological device can be irradiated with a natural UV source such as, for example, the sun, providing a range of wavelengths for irradiation.

In one aspect, culture of the biological device may proceed until the culture is dense, but not so dense as to trigger deleterious responses (e.g., a response triggered by lack of a food source) and not so dense as to prevent UV radiation from reaching a substantial portion of cells in the culture. Once the desired culture density has been reached, the culture can then be irradiated with UV radiation. Prior to irradiation, in one aspect, the culture is transferred to one or more vessels designed to allow a substantial portion of the biological device to be irradiated.

In one aspect, the irradiation continues for the length of time needed to induce a radiation response in the biological devices and ends at or before a time at which a substantial portion of the biological devices are fatally irradiated. In a further aspect, the extract can be collected after exposing a culture of a biological device to UV irradiation for a period of time ranging from about 12 hours to about 72 hours, or about 12, 24, 36, 48, 60, or 72 hours. In an alternative aspect, the biological devices may continue to be cultured for a time after UV exposure at least sufficient to allow some radiation response in the biological devices. In a further aspect, if irradiation did not cause the death of a substantial portion of the organisms in culture, culture may continue until the radiation response has ceased in a majority of the organisms.

In one aspect, radiation response can include upregulation of at least one of the following: a lipase, a zinc-related protein/oxidase, a silicatein, a silaffin, an alcohol dehydrogenase, or a combination thereof.

It will be understood that up-regulation or down-regulation of one or more of these proteins may not be directly responsible for UV-protective properties, such that increased or decreased amounts of these proteins in the extract may have little or no effect on the UV-protective properties of the extract. Further in this aspect, up-regulation or down-regulation of one of these proteins may have downstream effects that ultimately produce a UV-protective effect. In an alternative aspect, up-regulation or down-regulation of one or more of these proteins may be directly responsible for the UV-protective properties of the extract.

In one aspect, the extract is prepared in a manner able to isolate at least one UV-protective component. In some aspects, the extract can include centrifuged bacterial or yeast components. In one aspect, the extract is formulated at a variety of concentrations in any acceptable carrier to allow its use for a particular purpose. In some aspects, the extract is formulated in an evaporable carrier, such as water or alcohol, to allow the extract to dry on the surface of the material to be protected from UV radiation. In an alternative aspect, the extract is formulated in a lotion, gel, oil, or cream for application to human or animal skin.

In one aspect, the extract can be prepared by centrifuging the culture of biological devices in a manner able to precipitate most proteins, including UV-resistant and/or UV-protective proteins, then discarding the supernatant while retaining the pellet as the extract. Further in this aspect, the pellet can be used as-is or dried. Still further in this aspect, the pelleted material can be diluted to a given concentration using any acceptable carrier, such as water, alcohol, lotion, gel, oil, or cream. In one aspect, the carrier is non-denaturing. In an alternative aspect, the carrier is denaturing. In a still further aspect, the carrier also includes materials to inhibit further bacterial growth and/or protein degradation.

In an alternative aspect, the supernatant contains UV-protective compounds and is not discarded. In yet another aspect, the UV-protective and/or UV-resistant compounds and proteins are extracted by another method known in the art for isolating proteins and/or metabolites.

In a further aspect, the biological device culture may not be pelletized but instead may be killed, for example by lysis or exposure to lethal levels of UV radiation, and the culture medium can be used as-is or in an evaporated form. Further in this aspect, materials to inhibit further microorganism growth and/or protein degradation can also be introduced.

In another aspect, cells from any of the cultures described above can be isolated with or without extraction and/or lysis and used in wet or dry form.

In another aspect, isolated proteins from the biological device culture can be used in place of a more general extract to produce a UV-protective effect. Such proteins can be isolated by techniques known in the art.

In certain aspects, after culturing the biological device to produce the anti-microbial and UV-protective extracts, the host cells of the device can be lysed with one or more enzymes. For example, when the host cells are yeast, the yeast cells can be lysed with lyticase. In one aspect, the lyticase concentration can be 500, 600, 600, 800, 900, or 1000 μL per liter of culture, where any value can be the lower or upper endpoint of a range (e.g., 500 to 900 μL, 600 to 800 μL, etc.).

In addition to enzymes, other components can be used to facilitate lysis of the host cells. In one aspect, chitosan can be used in combination with an enzyme to lyse the host cells. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation. In one aspect, the chitosan is from 60% to about 100%, 80% to 90%, 75% to 85%, or about 80% acetylated. The molecular weight of the chitosan can vary, as well. For example, the chitosan can comprise about 3, 4, 5, 6, 7, 8, 9, 10, 11, 12, 13, 14, 15, 16, 17, 18, 19, or 20 glucosamine units and/or N-acetylglucosamine units. In another aspect, the chitosan includes 5 to 7 glucosamine units and/or N-acetylglucosamine units. In one aspect, chitosan can be added until a concentration of 0.0015, 0.0025, 0.005, 0.0075, 0.01, 0.015, 0.02, 0.03, 0.04, or 0.05% (where any value can be a lower or upper endpoint of a range, e.g., 0.005 to 0.02%, 0.0075 to 0.015%, etc.) is achieved in the culture. Still further in this aspect, the chitosan is present at a concentration of 0.01%.

In an alternative aspect, some or all of the chitosan can be replaced with the polyactive carbohydrate described herein.

In a further aspect, the anti-microbial and UV-protective extracts can be chemically-modified to produce additional desirable properties. Alternatively, compositions composed of the anti-microbial and UV-protective extracts with lysed and/or intact host cells (e.g., yeast) can be used herein, where it is not necessary to separate the host cells and other components from the anti-microbial and UV-protective extracts.

### IV. Applications of UV-Protective Extracts

The extract may be applied to any material that may benefit from a reduction in UV radiation. The exact formulation of the extract plus any carriers can be adjusted based on the desired use. In one aspect, the extract is formulated with only non-toxic components if it is to be used on a human or animal or with another microorganism, such as in a fermentation process or on an agricultural product. In another aspect, the extract can be mixed with other substances to provide UV-protective properties to the overall composition. In still another aspect, if coated on the material to be protected, the extract itself can be covered with a further protective coating to project, for example, against mechanical wear and damage.

In the case when the extract is applied to the surface of an article, it can be applied using techniques known in the art such as spraying or coating. In other aspects, the extract can be intimately mixed with a substance or material that ultimately produces the article. For example, the extract can be mixed with molten glass so that the extract is dispersed throughout the final glass product.

In one aspect, the extract is formulated or applied in such a manner as to block approximately 50%, 60%, 70%, 80%, 90%, 95%, or 99% of the UV radiation that encounters the extract, where any value can be a lower and upper end-point of a range (e.g., 60% to 95%). In a further aspect, the extract can also be formulated to block these percentages of particular UV wavelengths, or, more generally, to block these percentages of UVA, UVB, or UVC radiation.

Extracts according to the present disclosure can be used for a variety of purposes. These purposes include, but are not limited to, the following:
1. blocking UV radiation or other types of radiation;
2. protecting human skin against damage and/or skin cancer induced by UV radiation or other types of radiation;
3. protecting against side effects of radiation used in cancer treatments;
4. protecting animals from deleterious effects of UV radiation or other radiation;
5. protecting plastic, fiberglass, glass, rubber, or other solid surfaces from UV radiation or other radiation;
6. providing a UV radiation screen or screen for other types of radiation;
7. protecting astronauts and/or other persons or organisms as well as equipment during space trips;
8. enhancement of industrial fermentation processes or other processes requiring energy by allowing the use of UV radiation in connection with the process to supply additional energy and thus to increase the ultimate energy-requiring output of the cells without substantially killing the fermenting organism;
9. protection of experimentation, fermentation, biochemical, and/or biological processes under the presence of UV radiation, for example in extraterrestrial conditions such as on the moon or Mars; and
10. protection of agricultural plants, particularly agricultural plants in which the revenue-producing part of the plant is above ground, such as fruits, vine vegetables, beans and peas, and leaf vegetables.

In one particular embodiment, an extract prepared according to the procedure described above can be applied to an agricultural plant. In one aspect, the plant can be one that produces a fruit or vegetable, such as, for example, a watermelon or a tomato. Further in this aspect, the extract can be applied during at least a part of the plant's growth to increase the amounts of one or more nutrients of the fruit or vegetable, such as a vitamin, mineral, or other recommended dietary component. In one specific aspect, the amount of lycopene can be increased (which may be accompanied by a decrease in carotene or other less-valuable nutrients formed by competing pathways). In another aspect, the amount of a flavor-enhancing component, such as glucose, can be increased. Further in this aspect, an increase in glucose can help protect against water loss.

In one aspect, the extract can be applied for about 25%, 50%, 75%, 90%, 95%, or 99% of the fruit or vegetable's on-plant life, where the on-plant life includes the time span from the formation of a separate body that will constitute the fruit or vegetable (in some aspects, excepting flowers) until the fruit or vegetable is harvested. In one aspect, the extract can be first applied when the fruit or vegetable is sufficiently large to no longer be substantially protected from UV radiation by leaves. In another aspect, the extract can first be applied five days, one week, or two weeks prior to harvest. Further in this aspect, application at this later stage can be particularly useful with fruits or vegetables in which an increase in a nutrient or flavor-enhancing component can be obtained by protecting the fruit or vegetable from UV radiation later in its on-plant life.

In one aspect, the extract can be applied once or multiple times to each fruit or vegetable. In another aspect, it can be applied weekly, or it can be reapplied after the fruit or vegetable is exposed to rain or after a turning process. In another aspect, the agricultural plant can be another food crop that grows above ground and is exposed to natural UV radiation, wherein the agricultural product produced can be a fruit, leaf, seed, flower, grain, nut, stem, vegetable, or mushroom.

In another aspect, it is desirable for agricultural plants that do not produce parts typically consumed by humans to be protected from UV irradiation. In a further aspect, these other agricultural plants can includes sources of fibers such as, for example, cotton and linen (flax), of cork, of wood or lumber, of feedstocks for producing ethanol or biodiesel (including, but not limited to, sugar beet, sugarcane, cassava, sorghum, corn, wheat, oil palm, coconut, rapeseed, peanut, sunflower, soybean, and the like), of animal feedstocks or fodder, or of decorative or horticultural plants.

In one aspect, any part of the plant can be coated, including, but not limited to, the part of the plant that is collected during harvest. In an alternative aspect, the harvested part of the plant is not coated, but another part can be coated with the extracts disclosed herein. In addition to the aspects already described, in one aspect, coating a plant with the extracts described herein can prolong the life of the plant, increase production capacity of a desired product, can increase the growth rate of the plant relative to an untreated plant of the same type, can increase production of a desired metabolite that might otherwise decrease due to UV-induced stress, can increase yield of a crop of such plants, and the like.

In a further aspect, application can be accomplished with a commercial sprayer. In another aspect, application can be only on the upper portions of the fruit or vegetable, which are exposed to substantially greater amounts of UV radiation than the lower portions of the fruit or vegetable.

In another aspect, provided herein is a pharmaceutical composition containing the extracts produced by the biological devices described herein. In one aspect, the pharmaceutical composition can be applied to a subject, wherein the subject is exposed to radiation. In one aspect, the radiation is applied as a strategy to treat cancer. In another aspect, the pharmaceutical composition is used to prevent radiation-induced cellular and DNA damage. In another aspect, dosage ranges of the extract in the pharmaceutical composition can vary from 0.01 g extract/mL of pharmaceutical composition to 1 g extract/mL of pharmaceutical composition, or can be 0.01, 0.02, 0.025, 0.05, 0.075, or 1 g extract/mL of pharmaceutical composition. In an alternative aspect, provided herein is a cosmetic composition containing the extracts produced by the biological devices described herein. Further in this aspect, the cosmetic composition can be a cleanser, lotion, cream, shampoo, hair treatment, makeup, lip treatment, nail treatment, or related composition. In still a further aspect, the compositions containing the extracts can have both pharmaceutical and cosmetic applications. In yet another aspect, the compositions containing the extracts can be used in veterinary medicine.

The cosmetic compositions can be formulated in any physiologically acceptable medium typically used to formulate topical compositions. The cosmetic compositions can be in any galenic form conventionally used for a topical application such as, for example, in the form of dispersions of aqueous gel or lotion type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or alternatively multiple emulsions (W/O/VV or O/W/O), microemulsions, vesicular dispersions of ionic and/or non-ionic type, or wax/aqueous phase dispersions. These compositions are prepared according to the usual methods.

The cosmetic compositions can also contain one or more additives commonly used in the cosmetics field, such as emulsifiers, preservatives, sequestering agents, fragrances, thickeners, oils, waxes or film-forming polymers. In one aspect, in any of the above scenarios, the pharmaceutical, cosmetic, or veterinary composition also includes additional UV-protective compounds or UV-blocking agents such as, for example, zinc oxide, titanium dioxide, carotenoids, oxybenzone, octinoxate, homosalate, octisalate, octocrylene, avobenzone, or a combination thereof.

In one aspect, the composition is a sunscreen. A sunscreen can be formulated with any of the extracts produced herein. In addition to the extract, the sunscreen in certain aspects can be formulated with one or more UV-protective compounds or UV-blocking agents listed above. The sunscreen can be formulated as a paste, lotion, cream, aerosol, or other suitable formulations for topical use. In certain aspects, the sunscreen can be formulated as a transparent composition.

In one aspect, the cosmetic composition can be a film composed of the extracts produced herein that can be directly applied to the skin. For example, the film can be composed of a biocompatible material such as a protein or oligonucleotide, where the extract is coated on one or more surfaces of the film or, in the alternative dispersed throughout the film. For example, the film can be composed of DNA. In this application, the films can be used as a wound covering and provide protection from UV photodamage. The films can also be prepared so that they are optically transparent. Here, it is possible to view the wound without removing the covering and exposing the wound. The films can also include other components useful in cosmetic applications such as, for example, compounds to prevent or reduce wrinkles.

In one aspect, the pharmaceutical, cosmetic, or veterinary compositions described herein are applied to subjects. In one aspect, the subject is a human, another mammal, or a bird. In a further aspect, the mammal is a pet such as a dog or cat or is livestock such as horses, goats, cattle, sheep, and the like. In an alternative aspect, the bird is a pet bird or is poultry such as, for example, a chicken or turkey. In any of these aspects, the compositions can be applied to skin, fur, feathers, wool, hooves, horns, or hair as appropriate and applicable.

In another aspect, provided herein is a paint, dye, stain, or ink containing the UV-protective and/or UV-resistant extract disclosed herein. In one aspect, there are several benefits to having a paint that is resistant to UV irradiation. In a further aspect, imparting UV resistance to a paint slows or stops photodegradation, bleaching, or color fading. In another aspect, a paint with UV resistance prevents chemical modification of exposed paint surfaces. Further in this aspect, chemical modification of exposed paint surfaces includes change in finish, structural changes in binders, flaking, chipping, and the like. In one aspect, the paint provided herein resists these changes.

When the paint, ink, dye, or stain is applied to a surface, the anti-microbial and UV-protective extract can impart antifungal properties to the surface. In one aspect, applying a paint, ink, dye, or stain containing the anti-microbial and UV-protective extracts to the hull of a boat or other surface exposed to water can prevent or reduce the growth of barnacles. In another aspect, the anti-microbial and UV-protective extracts can be applied along with chitosan to the surface exposed to water.

In still another aspect, provided herein is an article coated with the extracts disclosed herein. In one aspect, the article is made of glass, plastic, metal, wood, fabric, or any combination thereof. In one aspect, the article is a construction material such as, for example, steel, concrete or cement, brick, wood, window or door glass, fiberglass, siding, wallboard, a flooring material, masonry, mortar, grout, stone, artificial stone, stucco, shingles, roofing materials, and the like. In a further aspect, the items and articles may be used in the construction and building industries in such applications as building materials, wood preservation, drywall, flooring, roofing materials and roofing membranes, artificial wood, plastic lumber, wood-filled plastics, decking, mobile homes, carpet, awnings, swimming pool liners, and related applications. In any of these applications, whether used as a coating or incorporated throughout the items and articles, the anti-microbial and UV-protective extracts can help preserve the items by reducing or eliminating the growth of fungus and/or other microbes.

In still another aspect, the items and articles may be home goods or consumer goods such as, for example, garments and textiles, leather, footwear and shoe soles, security documents, art and décor, cushions, mattresses, bath and/or kitchen mats, shower curtains, leisure furniture, plastic mulch, and the like.

In yet another aspect, the items and articles can be used in the transportation and automotive industries including upholstery for vehicles such as automobiles, trucks, trains, buses, and boats. In a further aspect, the items and articles can be used in various applications in the shipping industry such as packaging materials, crates, and pallets that are resistant to fungal colonization.

In an alternative aspect, the material is an aeronautical or aerospace material such as, for example, the metal or metal alloy body of an aircraft or spacecraft, paint on the body of an aircraft or spacecraft, glass windows on an aircraft or spacecraft, carbon fiber composite, titanium or aluminum, a ceramic heat absorbing tile, and the like. In still another aspect, the article is a fabric article such as, for example, clothing, drapes, outdoor upholstery, a tent or outdoor pavilion, a flag or banner, or the like. In another aspect, the extract can be applied to the article to fine artwork, solid pieces (e.g., vases), and historical documents in order to preserve them. In another aspect, the extract can be applied to outdoor signs such as highway billboards and advertising.

In other aspects, the extract can be incorporated within or throughout the article. In one aspect, the extract can be mixed with molten glass to produce glass article that are UV resistant such as, for example, sunglasses, car windshields, window glass, and eyeglasses. In another aspect, the glass article can be a bottle for storing a beverage or food container in order to increase the shelf-life of the beverage or food. It is contemplated that the extract can be applied externally to the glass articles as well.

In another aspect, the extract can be mixed with fiberglass or plastics in order to reduce negative effects to aircraft, watercraft, boats, jet skis, decking, house siding, motor homes, sunroofs, and moon roofs that are constantly exposed to UV radiation. It is contemplated that the extract can be applied externally to the fiberglass or plastic articles as well.

In another aspect, the extract can be mixed with rubber, silicone, or latex used to make a variety of articles such as water hoses, tires, and the like. It is contemplated that the extract can be applied externally to the rubber, silicone, or latex articles as well.

In another aspect, the extract can be mixed with foams used to make a variety of articles such as automotive dashboard padding, seat cushions, and the like. It is contemplated that the extract can be applied externally to the foam articles as well.

In another aspect, the extracts described herein can be incorporated into an optical film. In one aspect, the extract is applied to at least one surface of the film. In another aspect, the extract can be dispersed throughout the film. The film can be transparent, translucent or opaque. The film can be composed of, but not limited to, polyolefin resin, such as polyethylene (PE) or polypropylene (PP); polyester resin, such as polyethylene terephthalate (PET); polyacrylate resin, such as polymethyl (meth)acrylate (PMMA); polycarbonate resin; polyurethane resin or a mixture thereof. The optical film can be applied to any substrate where it is desirable to reduce or prevent UV exposure or damage. For example, the optical film can be applied to windows to reduce or prevent UV radiation from entering a structure (e.g., building, vehicle, etc.).

In still another aspect, the items and articles can be materials used in the manufacture of other goods. In this aspect, the items and articles can be plastic, coated fabrics, flexible films, foils or sheet, flexible extrusion products, products produced by injection molding, vinyl, gaskets, vinyl films or sheeting, plastisols, molded goods, or organosols. In yet another aspect, the items and articles can be artificial turf, parts such as, for example, filters used in air conditioning units, or materials intended for use in the oil and gas industries.

In another aspect, provided herein is a method of reducing or preventing the exposure of an item to UV radiation by applying the extracts described herein to the item or incorporating the extract within/throughout the article. Further in this aspect, “reducing” is defined relative to an untreated control. That is, if two like items are exposed to equal amounts of UV radiation for an equal amount of time, but one has been treated with the UV-resistant extracts and the other has not, and some objective response is measured (e.g., color fading, structural degradation, plant size or yield, etc.), the treated item will appear to have been exposed to a lower amount of UV (for example, the color of the treated item will have faded less and will remain closer to the original, or a treated plant will appear larger and more vigorous and will have a greater yield, etc.). In some aspects, treatment with the extracts disclosed herein will prevent UV exposure from occurring. As used herein, “prevent” indicates that a treated item will not be affected, changed, or altered by UV exposure.

In one aspect, the extract blocks from 50% to 100% of UV radiation from contacting the item. Further in this aspect, the extract blocks at least 50%, 55%, 60%, 65%, 70%, 75%, 80%, 85%, 90%, 95%, 99%, or 100% of UV radiation from contacting the item. In another aspect, the extract blocks from 50% to 100% of longwave UV radiation from contacting the item. Further in this aspect, the extract blocks at least 50%, 55%, 60%, 65%, 70%, 75%, 80%, 85%, 90%, 95%, 99%, or 100% of longwave UV radiation from contacting the item. In one aspect, the extract blocks from 50% to 100% of shortwave UV radiation from contacting the item. Further in this aspect, the extract blocks at least 50%, 55%, 60%, 65%, 70%, 75%, 80%, 85%, 90%, 95%, 99%, or 100% of shortwave UV radiation from contacting the item.

Depending upon the application, the extract can prevent or reduce damage cause by UV radiation from limited to extended periods of time. By varying the amount of extract that is applied as well as the number of times the extract is applied, the degree of UV protection can be varied. In certain aspects, it may be desirable for the article to be protected from UV damage for a short period of time then subsequently biodegrade.

In another aspect, the extracts produced herein can be used to reduce or prevent the growth of barnacles on boats and other water vehicles. In one aspect, the extract can be admixed with a paint that is typically applied to water vehicles, where the paint also includes chitosan. In one aspect, the chitosan can be acetylated to a specific degree of acetylation in order to enhance tissue growth during culturing as well as metabolite production. In one aspect, the chitosan is from 60% to about 100%, 70% to 90%, 75% to 85%, or about 80% acetylated. In one aspect, chitosan isolated from the shells of crab, shrimp, lobster, and/or krill is useful herein. The molecular weight of the chitosan can vary, as well. For example, the chitosan comprises about 3, 4, 5, 6, 7, 8, 9, 10, 11, 12, 13, 14, 15, 16, 17, 18, 19, or 20 glucosamine units and/or N-acetylglucosamine units. In another aspect, the chitosan includes 5 to 7 glucosamine units and/or N-acetylglucosamine units.

In all of the above aspects, incorporation of the anti-microbial and UV-protective extracts prevents or inhibits fungal growth, thereby reducing odors, improving and maintaining the appearance of the items and articles, reducing decomposition, and maintaining a microbe-free environment.

### V. UV-Resistant Plants

In one aspect, provided herein is a plant that is resistant to UV radiation. As used herein, “plant” is used in a broad sense to include, for example, any species of woody, ornamental, crop, cereal, fruit, or vegetable plant, as well as photosynthetic green algae. “Plant” also refers to a plurality of plant cells that are differentiated into a structure that is present at any stage of a plant's development. Such structures include, but are not limited to, fruits, shoots, stems, leaves, flower petals, roots, tubers, corms, bulbs, seeds, gametes, cotyledons, hypocotyls, radicles, embryos, gametophytes, tumors, and the like. “Plant cell,” “plant cells,” or “plant tissue” as used herein refers to differentiated and undifferentiated tissues of plants including those present in any of the tissues described above, as well as to cells in culture such as, for example, single cells, protoplasts, embryos, calluses, etc. It is contemplated that any cell from which a fertile plant can be regenerated is useful as a recipient cell. Type I, Type II, and Type III callus can be initiated from tissue sources including, but not limited to, immature embryos, immature inflorescences, seedling apical meristems, microspores, and the like. Those cells that are capable of proliferating as callus are also useful herein. Methods for growing plant cells are known in the art (see U.S. Pat. No. 7,919,679).

In one aspect, plant calluses grown from 2 to 4 weeks can be used herein. The plant cells can be derived from plants varying in age. The plant cells can be contacted with the biological device in a number of different ways. In one aspect, the device can be added to a medium containing the plant cells. In another aspect, the device can be injected into the plant cells via syringe. The amount of device and the duration of exposure to the device can vary as well. In one aspect, the concentration of the device is about 10³, 10⁴, 10⁵, 10⁶, 10⁷, 10⁸, or 10⁹ cells/mL of water. In one aspect, when the host cell is a bacterium, the concentration of the device is about 10⁶. In another aspect, when the host cell is yeast, the concentration of the device is about 10⁹. Different volumes of the biological device can be used as well, ranging from 5 μL to 500 μL.

In certain aspects, any of the biological devices described above can be used in combination with a polysaccharide to enhance one or more physiological properties of the plant. In one aspect, the plant cells are first contacted with the biological device, then subsequently contacted with the polysaccharide. In another aspect, the plant cells are first contacted with the polysaccharide and subsequently contacted with the biological device. In a further aspect, the plant cells are simultaneously contacted with the polysaccharide and the biological device.

In one aspect, the polysaccharide includes chitosan, glucosamine (GlcN), N-acetylglucosamine (NAG), or any combination thereof. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation in order to enhance tissue growth during culturing as well as metabolite production. In one aspect, the chitosan is from 60% to about 100%, 70% to 90%, 75% to 85%, or about 80% acetylated. In one aspect, chitosan isolated from the shells of crab, shrimp, lobster, and/or krill is useful herein.

The molecular weight of the chitosan can vary, as well. For example, the chitosan comprises about 3, 4, 5, 6, 7, 8, 9, 10, 11, 12, 13, 14, 15, 16, 17, 18, 19, or 20 glucosamine units and/or N-acetylglucosamine units. In another aspect, the chitosan includes 5 to 7 glucosamine units and/or N-acetylglucosamine units. In one aspect, the chitosan is in a solution of water and acetic acid at less than 1% by weight, less than 0.75% by weight, less than 0.5% by weight, less than 0.25% by weight, or less than 0.1% by weight. In another aspect, the amount of chitosan that is applied to the plant cells is from 0.1% to 0.01% by weight, from 0.075% to 0.025% by weight, or is about 0.05% by weight. The polysaccharides used herein are generally natural polymers and thus present no environmental concerns. Additionally, the polysaccharide can be used in acceptably low concentrations.

In an alternative aspect, the polyactive carbohydrate and/or carbo sugars disclosed herein can be used in place of some or all of the polysaccharide.

The plant cells can be contacted with the polysaccharide using a number of techniques. In one aspect, the plant cells or reproductive organs (e.g., a plant embryo) can be cultured in agar and medium with a solution of the polysaccharide. In other aspects, the polysaccharide can be applied to a plant callus by techniques such as, for example, coating the callus or injecting the polysaccharide into the callus. In this aspect, the age of the callus can vary depending upon the type of plant. The amount of polysaccharide can vary depending upon, among other things, the selection and number of plant cells. The use of the polysaccharide in the methods described herein permits rapid tissue culturing at room temperature. Due to the ability of the polysaccharide to prevent microbial contamination, the tissue cultures can grow for extended periods of time ranging from days to several weeks. Moreover, tissue culturing with the polysaccharide can occur in the dark and/or light. As discussed above, the plant cells can be contacted with any of the biological devices described above. Thus, the use of the polysaccharides and biological devices described herein is a versatile way to culture and grow plant cells—and ultimately, plants of interest—with enhanced physiological properties.

In one aspect, a plant callus such as described above can be planted and allowed to grow and mature into a plant bearing fruit and leaves.

In a further aspect, provided herein is a plant grown by the process of contacting plant gamete cells, a plant reproductive organ, or a plant callus with the biological devices disclosed herein. Also provided herein is a method for producing such a plant. In one aspect, the method includes the steps of:
(a) contacting a plant callus with the biological device;
(b) culturing the plant callus; and
(c) growing a plant from the plant callus.

In some aspects, the plant callus is cultured with chitosan. In a further aspect, the chitosan is from 60%, 65%, 70%, 75%, 80%, 85%, 90%, 95%, or 100% acetylated and has 3, 4, 5, 6, 7, 8, 9, 10, 11, 12, 13, 14, 15, 16, 17, 18, 19, or 20 glucosamine units, N-acetylglucosamine units, or a combination thereof, where any value can be a lower and upper end-point of a range (e.g., 60% to 80% acetylation).

In another aspect, provided herein is a method for increasing the UV-resistance of a plant, the method involving growing a plant from plant cells that have been contacted with the biological devices disclosed herein. In one aspect, increased UV-resistance can be measured by growing plants from a treated and an untreated callus alongside one another and comparing UV-induced damage after a period of time. In a further aspect, an agricultural product harvested from a UV-resistant plant will also be more UV-resistant. Further in this aspect, for example, cotton from a cotton plant grown with the biological devices will be more UV-resistant than cotton grown from an untreated plant.

### VI. Production of Hard and Soft Biofoams and Articles Made Therefrom

In another aspect, the anti-microbial and UV-protective extracts described herein can be used to produce polyurethane compositions that have numerous applications.

In one aspect, the polyurethane composition is produced by:
a) admixing a polyactive carbohydrate and a natural oil polyol to produce a first admixture; and
b) admixing the first admixture with a polyisocyanate to produce the polyurethane composition.

In another aspect, the polyurethane composition is produced by:
a) admixing a carbo sugar and a natural oil polyol to produce a first admixture; and
b) admixing the first admixture with a polyisocyanate to produce the polyurethane composition.

In still another aspect, the polyurethane composition is produced by:
a) admixing a polyactive carbohydrate, a carbo sugar, and a natural oil polyol to produce a first admixture; and
b) admixing the first admixture with a polyisocyanate to produce the polyurethane composition.

In another aspect, the polyurethane composition produced by:
a) admixing the extract with a polyactive carbohydrate, and a natural oil polyol to produce a first admixture; and
b) admixing the first admixture with a polyisocyanate and a carbo sugar to produce the polyurethane composition.

In one aspect, the polyurethane composition is produced by:
a) admixing the extract with a polyactive carbohydrate and castor oil to produce a first admixture; and
b) admixing the first admixture with a polyisocyanate, a carbo sugar, and beeswax to produce the polyurethane composition,
where the polyactive carbohydrate comprises an extract produced from host cells comprising a DNA construct comprising the following genetic components: (a) a gene that expresses chitin synthase, (b) a gene that expresses chitosanase, and (c) a gene that expresses chitin deacetylase and the carbo sugar comprises an extract produced from host cells comprising the following genetic components: (1) a gene that expresses cellulose synthase and (2) a gene that expresses galactomannan galactosyltransferase, and
the polyisocyanate is an aromatic diisocyanate such as, for example, 4,4′-methylene diphenyl diisocyanate.

In one aspect, the anti-microbial and UV-protective extracts disclosed herein can be added at any step in the method for producing the polyurethane compositions. Further in this aspect, the extracts can be added at the time of mixing the polyactive carbohydrate and/or carbo sugar and natural oil polyol, or can be present in the solutions of polyactive carbohydrate, carbo sugar, and/or natural oil polyol prior to mixing, or can be added at the time of mixing the first admixture with the polyisocyanate. In an alternative aspect, the extracts can be used to coat items or articles made from the polyurethane compositions.

As used herein, a “carbo sugar” is a polymer with an oligo- or poly-mannose backbone that is fully or partially galactosylated. In one aspect, the carbo sugar can be chemically or enzymatically fully or partially hydrolyzed prior to use in order to fine tune the molecular weight and associated properties of the carbo sugar. In one aspect, the carbo sugar is produced by a biological. In another aspect, the carbo sugar can be produced by DNA constructs shown in FIGS. 5A-5D transformed in host cells (e.g., yeast, bacteria) and subsequently cultured to yield a carbo sugar extract. The carbo sugar extract can then be isolated using the techniques described herein.

As used herein, a “polyactive carbohydrate” is a polymer composed of individual monosaccharide units. In one aspect, the polyactive carbohydrate is partially or fully acetylated. In another aspect, an enzymatic or chemical deacetylation process can be used on the polyactive carbohydrate or any precursors to alter the degree of acetylation. In still another aspect, the polyactive carbohydrate can be chemically or enzymatically fully or partially hydrolyzed prior to use in order to fine tune the properties of the polyactive carbohydrate as they relate to molecular weight. In one aspect, the polyactive carbohydrate is produced by a biological device. In another aspect, the polyactive carbohydrate can be produced by DNA constructs shown in FIGS. 3A-3B and 4A-4B transformed in host cells (e.g., yeast, bacteria) and subsequently cultured to yield the polyactive carbohydrate. The polyactive carbohydrate extract can then be isolated using the techniques described herein.

A “natural oil” as used herein is any oil extracted from a living organism. In one aspect, the living organism is a plant or alga. In a further aspect, the plant is the castor bean or castor oil plant (Ricinus communis). In another aspect, the living organism is an animal. In an alternative aspect, the living organism is a fungus. Natural oils can additionally contain triglycerides, fatty acids, fatty acid esters, sterols, isoprenoid or terpenoid compounds, alkaloids, phenols, and other metabolites.

“Natural oil polyols” are compounds that include at least one free hydroxyl group and are derived from or present in natural oils. A natural oil polyol may be naturally occurring, as with the ricinoleic acid in castor oil, or it may be chemically synthesized from an oil or fat containing one or more carbon-carbon double bonds. In one aspect, a natural fatty acid or triglyceride containing a carbon-carbon double bond is subjected to ozonolysis to cleave the double bond, followed by treatment with another molecule such as, for example, ethylene glycol, to form an alcohol. In another aspect, a natural fatty acid or triglyceride containing a carbon-carbon double bond can be formylated in the presence of carbon monoxide and hydrogen gas, followed by hydrogenation to produce a hydroxyl group. Other methods of producing natural oil polyols are also contemplated. Natural oils can be used as extracted or can optionally be purified. In one aspect, the natural oil polyol is or is derived from soy, a chemically-modified vegetable oil, a carbohydrate, lignin, cork, cashew nutshell liquid, Lesquerella oil, or a combination thereof. In one aspect, the natural oil polyol is castor oil. In another aspect, the natural oil polyol is ricinoleic acid. In still another aspect, the natural oil polyol is coriolic acid or a chemically-modified fatty acid.

“Castor oil” can optionally be extracted from the seeds of the castor oil plant. The primary component of castor oil is ricinoleic acid; minor components include oleic acid, linoleic acid, linolenic acid, stearic acid, palmitic acid, dihydroxystearic acid, and other trace fatty acids.

In one aspect, the natural oil polyol can include one or more hydroxyl fatty acids, which are defined herein as fatty acids having at least one free hydroxyl group. The hydroxyl fatty acid has the general formula R′C(O)OH, wherein R′ is a saturated or unsaturated hydrocarbon chain having from 10 to 25 carbon atoms, and at least one hydroxyl group is covalently attached to a carbon atom of the hydrocarbon chain. The hydrocarbon can be linear or branched. In the case where the hydrocarbon is unsaturated, the hydrocarbon can have one carbon-carbon double bond or multiple carbon-carbon double bonds. Examples of monohydroxy fatty acids (i.e., one hydroxyl group present on the fatty acid) include, but are not limited to, hydroxynervonic acid, cerebronic acid, 10-hydroxy-20-decenoic acid, hydroxyl-2-decenoic acid 10-phoshpate, strophantus acid, lesquerolic acid, densipolic acid, auricolic acid, α-dimorphecolic acid, kamlolenic acid, 8-hydroxyoctadeca-9,11-diynoic acid, 8-hydroxyoctadeca-17-en-9,11-diynoic acid (isanolic), or 8-hydroxyoctadeca-13,17-dien-9,11-diynoic acid. Examples of polyhydroxy fatty acids (i.e., two or more hydroxyl groups) include, but are not limited to, axillarenic acid, tetrapedic acid, byrsonic acid, 9,10-dihydroxyoctadecanoic acid, phaseolic acid, phloionolic acid, Resolvin D1, 10,18S-docosatriene, or Resolvin E1. The hydroxyl fatty acids can be used as is in the natural oil (e.g., castor oil), isolated from a natural oil, or synthesized accordingly.

In certain aspects, a surfactant can be used to produce the polyurethane compositions described herein, where the surfactant is admixed with the polyactive carbohydrate and/or carbo sugar and a natural oil polyol to produce a first admixture. The anti-microbial and UV-protective extracts may be added to the admixture during this step or to one of the reagents prior to admixing to produce the first admixture. A “surfactant” is an organic compound that may be derived from a natural product, or may result from chemical modification of a natural product, or may be completely chemically synthesized. Surfactants typically contain hydrophilic head groups and hydrophobic tails. In one aspect, the head group is anionic, cationic, non-ionic, or zwitterionic. In another aspect, the tail is composed of a hydrocarbon or a glucoside. Surfactants alter the surface tension of liquids and may form micelles or bilayers in aqueous solution. Many applications of surfactants are known in the art. Surfactants are, for example, commonly employed as emulsifiers, detergents, wetting agents, and the like.

Numerous cationic surfactants can be used in the compositions described herein. In one aspect, the cationic surfactant can be a quaternary ammonium salt.

Numerous zwitterionic surfactants can be used in the compositions described herein. In one aspect, the zwitterionic surfactant can be a lecithin such as soy lecithin; in another aspect, the zwitterionic surfactant can be a hydroxysultaine, a betaine, a sulfobetaine, or a mixture thereof. Among betaines, surfactants may be selected from the group comprising high alkyl betaines such as cetyl dimethyl carboxymethyl betaine, cocamidopropyl betaine, cocobetaine, coco dimethyl carboxymethyl betaine, lauryl amidopropyl betaine, lauryl bis-(2-hydroxyethyl) carboxymethyl betaine, lauryl bis-(2-hydroxypropyl) alpha-carboxyethyl betaine, lauryl dimethyl alphacarboxyethyl betaine, lauryl dimethyl carboxymethyl betaine, oleyl betaine, oleyl dimethyl gamma-carboxypropyl betaine, stearyl bis-(2-hydroxypropyl) carboxymethyl betaine, and mixtures thereof. Among sulfobetaines, surfactants may be selected from the group comprising coco dimethyl sulfopropyl betaine, lauryl bis-(2-hydroxyethyl) sulfopropyl betaine, lauryl dimethyl sulfoethyl betaine, stearyl dimethyl sulfopropyl betaine, and mixtures thereof. Amidobetaines and amidosulfobetaines are also contemplated.

Numerous nonionic surfactants can be used in the compositions described herein. Nonionic surfactants useful in the compositions described herein include alkoxylated fatty acid esters, alkyl glucosides, alkyl polyglucosides, amine oxides, alcohol ethoxylates, cocoamine oxide, glyceryl monohydroxystearate, glyceryl stearate, hydroxyl stearic acid, lauramine oxide, laureth-2, polyhydroxy fatty acid amides, polyoxyalkylene stearates, propylene glycol stearate, sorbitan monostearate, sucrose cocoate, sucrose esters, sucrose laurate, steareth-2, PEG-40 hydrogenated castor oil, and mixtures thereof. Preferred nonionic surfactants include those based on polyethoxylated sorbitan and oleic acid such as, for example, polysorbate 80 and polysorbate 20, both of which are available under a variety of trade names.

Further nonionic surfactants contemplated include, in one aspect, the condensation products of a higher aliphatic alcohol, such as a fatty alcohol, containing about 8 to 20 carbon atoms, in a straight or branched chain configuration, condensed with about 3 to about 100 moles, preferably about 5 to about 40 moles, most preferably about 5 to about 20 moles of ethylene oxide. Examples of such nonionic ethoxylated fatty alcohol surfactants are the Tergitol™ 15-S series from Union Carbide and Brij™ surfactants from ICI. Tergitol™ 15-S surfactants include C₁₁-C₁₃ secondary alcohol polyethylene glycol ethers, Brij™ 97 surfactant is polyoxyethylene(10) oleyl ether; Brij™58 surfactant is polyoxyethylene(20) cetyl ether; and Brij™ 76 surfactant is polyoxyethylene(10) stearyl ether.

In another aspect, a useful class of nonionic surfactants includes the polyethylene oxide condensates of one mole of alkyl phenol containing from about 6 to 12 carbon atoms in a straight or branched chain configuration, with about 3 to about 100 moles, preferably about 5 to about 40 moles, most preferably about 5 to about 20 moles of ethylene oxide to achieve the above defined HLB. Examples of nonreactive nonionic surfactants are the Igepal™ CO and CA series from Rhone-Poulenc. Igepal™ CO surfactants include nonylphenoxy poly(ethyleneoxy) ethanols. Igepal™ CA surfactants include octylphenoxy poly(ethyleneoxy) ethanols. Still another useful class of hydrocarbon nonionic surfactants includes block copolymers of ethylene oxide and propylene oxide or butylene oxide with HLB values of about 6 to about 19, preferably about 9 to about 18, and most preferably about 10 to about 16. Examples of such nonionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers. In other aspects, the nonionic surfactants include sorbitan fatty acid esters, polyoxyethylene sorbitan fatty acid esters and polyoxyethylene stearates having HLBs of about 6 to about 19, about 9 to about 18, and about 10 to about 16. Examples of such fatty acid ester nonionic surfactants are the Span™, Tween™, and Myj™ surfactants from ICI. Span™ surfactants include C₁₂-C₁₈ sorbitan monoesters. Tween™ surfactants include poly(ethylene oxide) C₁₂-C₁₈ sorbitan monoesters. Myj™ surfactants include poly(ethylene oxide) stearates. In one aspect, the nonionic surfactant can include polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ether, polyoxyethylene cetyl ether, polyoxyethylene stearyl ether, polyoxyethylene oleyl ether, polyoxyethylene octylphenyl ether, polyoxyethylene nonylphenyl ether, polyethylene glycol laurate, polyethylene glycol stearate, polyethylene glycol distearate, polyethylene glycol oleate, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurate, polyoxyethylene sorbitan stearate, polyoxyethylene sorbitan oleate, polyoxyethylene laurylamine, polyoxyethylene laurylamide, laurylamine acetate, hard beef tallow propylenediamine dioleate, ethoxylated tetramethyldecynediol, fluoroaliphatic polymeric ester, polyether-polysiloxane copolymer, and the like.

Numerous anionic surfactants can be used herein. In one aspect, the anionic surfactant can be alcohol phosphates and phosphonates, alkyl alkoxy carboxylates, alkyl aryl sulfates, alkyl aryl sulfonates, alkyl carboxylates, alkyl ether carboxylates, alkyl ether sulfates, alkyl ether sulfonates, alkyl phosphates, alkyl polyethoxy carboxylates, alkyl polyglucosides, alkyl polyglucoside sulfates, alkyl polyglucoside sulfonates, alkyl succinamates, alkyl sulfates, alkyl sulfonates, aryl sulfates, aryl sulfonates, fatty taurides, isethionates, N-acyl taurates, nonoxynol phosphates, octoxynol phosphates, sarcosinates, sulfated fatty acid esters, taurates, and mixtures thereof. Useful anionic surfactants include, but are not limited to, alkali metal and (alkyl) ammonium salts of: 1)alkyl sulfates and sulfonates such as sodium dodecyl sulfate, sodium 2-ethylhexyl sulfate, and potassium dodecanesulfonate; 2) sulfates of polyethoxylated derivatives of straight or branched chain aliphatic alcohols and carboxylic acids; 3)alkylbenzene or alkylnaphthalene sulfonates and sulfates such as sodium laurylbenzene-4-sulfonate and ethoxylated and polyethoxylated alkyl and aralkyl alcohol carboxylates; 5)glycinates such as alkyl sarcosinates and alkyl glycinates; 6) sulfosuccinates including dialkyl sulfosuccinates; 7) isothionate derivatives; 8) N-acyltaurine derivatives such as sodium N methyl-N-oleyltaurate); 9)amine oxides including alkyl and alkylamidoalkyldialkylamine oxides; and 10)alkyl phosphate mono or di-esters such as ethoxylated dodecyl alcohol phosphate ester, sodium salt. Representative commercial examples of suitable anionic sulfonate surfactants include, for example, sodium lauryl sulfate, available as TEXAPON™ L-100 from Henkel Inc., Wilmington, Del., or as POLYSTEP™ B-3 from Stepan Chemical Co, Northfield, Ill.; sodium 25 lauryl ether sulfate, available as POLYSTEP™ B-12 from Stepan Chemical Co., Northfield, Ill.; ammonium lauryl sulfate, available as STANDAPOL™. A from Henkel Inc., Wilmington, Del.; and sodium dodecyl benzene sulfonate, available as SIPONATE™ DS-10 from Rhone-Poulenc, Inc., Cranberry, N.J., dialkyl sulfosuccinates, having the trade name AEROSOL™ OT, commercially available from Cytec Industries, West Paterson, N.J.; sodium methyl taurate (available under the trade designation NIKKOL™ CMT30 from Nikko Chemicals Co., Tokyo, Japan); secondary alkane sulfonates such as Hostapur™ SAS which is a Sodium (C14-C17) secondary alkane sulfonates (alpha-olefin sulfonates) available from Clariant Corp., Charlotte, N.C.; methyl-2-sulfoalkyl esters such as sodium methyl-2-sulfo (C12-16)ester and disodium 2-sulfo (C12-C16) fatty acid available from Stepan Company under the trade designation ALPHASTE™ PC48; alkylsulfoacetates and alkylsulfosuccinates available as sodium laurylsulfoacetate (under the trade designation LANTHANOL™ LAL) and disodiumlaurethsulfosuccinate (STEPANMILD™ SL3), both from Stepan Company; alkylsulfates such as ammoniumlauryl sulfate commercially available under the trade designation STEPANOL™ AM from Stepan Company, and/or dodecylbenzenesulfonic acid sold under BIO-SOFT AS-100 from Stepan Chemical Co. In one aspect, the surfactant can be a disodium alpha olefin sulfonate, which contains a mixture of C₁₂ to C₁₆ sulfonates. In one aspect, CALSOFT™ AOS-40 manufactured by Pilot Corp. can be used herein as the surfactant. In another aspect, the surfactant is DOWFAX 2A1 or 2G manufactured by Dow Chemical, which are alkyl diphenyl oxide disulfonates. Representative commercial examples of suitable anionic phosphate surfactants include a mixture of mono-, di- and tri-(alkyltetraglycolether)-o-phosphoric acid esters generally referred to as trilaureth-4-phosphate commercially available under the trade designation HOSTAPHAT™ 340KL from Clariant Corp., as well as PPG-5 cetyl 10 phosphate available under the trade designation CRODAPHOS™ SG from Croda Inc., Parsipanny, N.J. Representative commercial examples of suitable anionic amine oxide surfactants those commercially available under the trade designations AMMONYX™ LO, LMDO, and CO, which are lauryldimethylamine oxide, laurylamidopropyldimethylamine oxide, and cetyl amine oxide, all from Stepan Company.

In one aspect, a surfactant is chosen based on its ability to form a stable emulsion containing an acidic aqueous solution of a polyactive carbohydrate and/or a carbo sugar, a natural oil polyol, and, in some aspects, the anti-microbial and UV-protective extracts described herein. In other aspects, the anti-microbial and UV-protective extracts are added later and the surfactant is not required to form a stable emulsion containing the anti-microbial and UV-protective extracts. In a further aspect, the concentration of surfactant can be from 0.001% to 1% (v/v), or can be about 0.001%, 0.005%, 0.01%, 0.05%, 0.1%, 0.35%, 0.5%, or 1% (v/v) with respect to the final emulsion volume. In another aspect, 0.35% of polysorbate 80 is used. In a further aspect, emulsion formation can be evaluated as a function of stirring time (e.g., about 1 minute, about 2 minutes, about 4 minutes, about 6 minutes, about 8 minutes, or about 10 minutes) and/or stirring speed (e.g., about 2000 rpm, about 5000 rpm, about 10,000 rpm, or about 20,000 rpm).

In another aspect, beeswax can be used as a component to produce the polyurethane and biofoam. Any type of beeswax can be used in this embodiment (e.g., European, Oriental). Beeswax is typically composed of a mixture of hydrocarbons, esters (mono, di, tri), hydroxy esters and polyesters, free fatty acids, and free fatty alcohols, with one of the main components being triacontanyl palmitate. The beeswax cab e added at any stage during production of the polyurethane composition. In one aspect, the beeswax is admixed with the composition composed of the anti-microbial and UV-protective extracts disclosed herein with the polyactive carbohydrate and/or carbo sugar. In another aspect, the beeswax is admixed with the polyisocyanate.

The order in which the components can be admixed with one another to produce the first admixture can vary. In one aspect, the natural oil polyol can be added to a solution of the polyactive carbohydrate and/or the carbo sugar. In one aspect, the natural oil polyol is added over time (e.g., 2 minutes, 4 minutes, 5 minutes, 6 minutes, 8 minutes, or 10 minutes) with stirring (2000 rpm, 5000 rpm, 10,000 rpm, or 20,000 rpm) to create a final admixture that also incorporates the polyactive carbohydrate and/or the carbo sugar. In one aspect, the natural oil polyol is castor oil and stirring is conducted at 10,000 rpm for 5 minutes. In any of these aspects, the anti-microbial and UV-protective extracts disclosed herein can be added at any step of the process, to any solution or reagent.

In one aspect, the carbo sugar, if used, is from 0.1 to 1% by weight of the first admixture. In another aspect, the amount of carbo sugar is 0.1, 0.2, 0.3, 0.4, 0.5, 0.6, 0.7, 0.8, 0.9, or 1 wt % of the first admixture, where any value can be a lower or upper endpoint of a range (e.g., 0.2 to 0.7, etc.). In another aspect, the carbo sugar can be prepared and used as a solution. In one aspect, the carbo sugar is an aqueous solution of 1% to 5% (w/v), wherein the first admixture includes 20% to 80% (v/v) of the aqueous solution of carbo sugar.

In one aspect, the polyactive carbohydrate is from 0.1 to 1% by weight of the first admixture. In another aspect, the amount of polyactive carbohydrate is 0.1, 0.2, 0.3, 0.4, 0.5, 0.6, 0.7, 0.8, 0.9, or 1 wt % of the first admixture, where any value can be a lower or upper endpoint of a range (e.g., 0.2 to 0.7, etc.). In another aspect, the polyactive carbohydrate can be prepared and used as a solution. In one aspect, the polyactive carbohydrate is an aqueous solution of 1% to 5% (v/v), where the first admixture includes 20% to 80% (v/v) of the aqueous solution of the polyactive carbohydrate.

In one aspect, the natural oil polyol is from 20% to 80% (v/v) of the first admixture. In another aspect, the natural oil polyol is about 20%, 25%, 30%, 35%, 40%, 45%, 50%, 55%, 60%, 70%, 75%, or 80% (v/v) of the first admixture, where any value can be a lower or upper endpoint of a range (e.g., 40% to 60%, etc.).

Prior to the addition of the polyisocyanate, additional components can be added to the first admixture of polyactive carbohydrate and/or carbo sugar and natural oil polyol. In one aspect, a catalyst can be added to the first admixture. A “catalyst” as used herein is any substance that can increase the rate of a chemical reaction. In one aspect, the catalyst is not consumed in the reaction. A single molecule of a catalyst can assist with multiple chemical reactions. Catalysts useful herein include, but are not limited to, tertiary amines such as dimethylethanolamine (DMAE), triethylenediamine (DABCO), 3-aminopropyldimethylamine (DMAPA), dimethylcyclohexylamine (DMCHA); compounds containing hydroxyl groups or secondary amines such as, for example, propylene glycol; metallic compounds including metal carboxylates such as, for example, dibutyltin dilaurate (DBTDL) as well as mercury, lead, bismuth, and zinc carboxylates; and other alkyl tin carboxylates, oxides, and mercaptides. In one aspect, the catalyst is added to an emulsion containing the polyactive carbohydrate, natural oil polyol, and, in some aspects, the anti-microbial and UV resistant extracts at from about 0.05% to about 2% (v/v) with respect to the volume of the emulsion. In another aspect, about 0.05%, 0.1%, 0.15%, 0.2%, 0.25%, 0.4%, 0.6%, 0.7%, 0.8%, 1%, 1.2%, 1.5%, or 2% catalyst is used. In some aspects, a combination of catalysts is used. In one aspect, 0.5% (v/v) dibutyltin dilaurate and 1% (v/v) dimethylethanolamine were used in combination. In a further aspect, stirring is used to incorporate the catalyst throughout an emulsion containing the polyactive carbohydrate and/or the carbo sugar, the natural oil polyol, and, in some aspects, the anti-microbial and UV resistant extracts disclosed herein. In one aspect, different stirring times (e.g., about 1 minute, about 2 minutes, about 3 minutes, about 4 minutes, about 5 minutes, about 8 minutes, or about 10 minutes) and different stirring speeds (about 100 rpm, about 200 rpm, about 300 rpm, about 40 rpm, about 500 rpm, about 600 rpm, or about 700 rpm) are evaluated to determine the minimum stirring time and speed required to fully incorporate the catalyst into the emulsion. In one aspect, the emulsion and added catalyst are stirred at 300 rpm for 3 minutes.

In another aspect, a clay can be added to the first admixture. “Clay” and “clay minerals” as used herein refer to hydrous aluminum phylosilicates. Clays can optionally include oxides and/or chelates of other metals and semimetals such as, for example, silicon, iron, calcium, magnesium, sodium, potassium, and other alkali and alkaline earth metals. “Bentonite” is a category of impure clay that can consist of montmorillonite, kaolinite, and other species; and that can include potassium, sodium, calcium, aluminum, and/or other metals. “Zeolites” are microporous aluminosilicates that can accommodate a variety of cations, including, but not limited to, potassium, calcium, and magnesium. The cations in zeolites can be exchanged in aqueous solutions. Clays, bentonites, and zeolites can be used as sources of trace oxides and/or ions in the practice of the present invention. An “oxide” as used herein refers to a molecule, a network solid, or an ionic compound containing at least one oxygen atom and one other element. In one aspect, clays, bentonites, and zeolites contain chelated metal and semimetal ions. Not wishing to be bound by theory, the inclusion of the clay can be used to vary the pore size of the final biofoam product produced.

In one aspect, a metal or semimetal oxide or a chelated metal ion can be incorporated into the first admixture. In one aspect, the metal or semimetal oxide includes, for example, Al₂O₃, Fe₂O₃, MgO, CaO, Na₂O, K₂O, SiO₂, or a combination thereof. In this aspect, the metal or semimetal oxide can be introduced into the polyurethane compositions as a pure compound. In an alternative aspect, ions such as, for example, aluminum, iron (III), magnesium, calcium, sodium, potassium, silicon, and combinations thereof, can be incorporated into the polyurethane compositions described herein through the inclusion of clays or clay minerals. In one aspect, the metal or semimetal oxides or chelated metals are incorporated at concentrations of from about 0.02 nM to about 1.2 mM, or at 0.02 nM, 0.04 nM, 0.06 nM, 0.08 nM, 0.1 nM, 0.15 nM, 0.2 nM, 0.25 nM, 0.3 nM, 0.35 nM, 0.4 nM, 0.45 nM, 0.5 nM, 0.55 nM, 0.2 mM, 0.4 mM, 0.6 mM, 0.8 mM, or 1.2 mM.

In another aspect, one or more water-soluble metal salts can be incorporated into the first admixture. In one aspect, the water-soluble metal salts can include, for example, gallium (III) nitrate hydrate, zinc sulfate, zinc acetate, or a combination thereof. In one aspect, 50 mg/L of gallium (III) nitrate hydrate is incorporated into the emulsion containing the polyactive carbohydrate, natural oil polyol, and the anti-microbial and UV-protective extracts. In another aspect, 100 mg/L of zinc sulfate is incorporated into the emulsion containing the polyactive carbohydrate, natural oil polyol, and in some aspects, the anti-microbial and UV-protective extracts.

After preparation of the first admixture as described above, a polyisocyanate is added to the first admixture. “Polyisocyanates” as used herein are compounds with two or more —N═C═O groups. In one aspect, the polyisocyanate is an aliphatic diisocyanate, a cycloaliphatic diisocyanate, an aromatic diisocyanate, or an isomer thereof. In another aspect, the isocyanate or polyisocyanate is 2,4-toluenediisocyanate, 2,6-toluenediisocyanate, 4,4′-methylene diphenyl diisocyanate (MDI), 4,4′-methylenebis(cyclohexylisocyanate) (H12-MDI), 1-isocyanato-3-isocyanato-methyl-3,5,5-trimethylcyclohexane (isophorone diisocyanate), 2,4,4-trimethylhexamethylenediisocyanate, ethylidenediisocyanate, butylenediisocyanate, hexamethylenediisocyanate, cyclohexylene-1,4-diisocyanate, cyclohexylene-1,2-diisocyanate, xylylene diisocyanate, dichlorohexamethylene diisocyanate, dicyclohexyl-4,4′-diisocyanate, 1-methyl-2,4-diisocyanato-cyclohexane, 1-methyl-2,6-diisocyanato-cyclohexane, naphthalene-1,5-diisocyanate, p-phenylendiisocyanate, tetramethyl-xylylenediisocyanate (TMXDI), or any combination thereof. The isocyanate or polyisocyanate can exist as one or more structural isomers. Alternatively, the isocyanate or polyisocyanate can be a dimer, trimer, or oligomer. In other aspects, the isocyanate or polyisocyanate can exist as one or more positional isomers. For example, the polyisocyanate can be a mixture of 2,4-toluenediisocyanate and 2,6-toluenediisocyanate. In a further aspect, the polyisocyanate can be a 65:35 mixture of 2,4-TDI and 2,6-TDI (i.e., TDI 65). In a different aspect, the polyisocyanate can be an 80:20 mixture of 2,4-TDI and 2,6-TDI (i.e., TDI 80). In an alternative aspect, the polyisocyanate is a modified MDI or polyphenylmethane polyisocyanate such as one of those sold by Yantai Wanhua Polyurethanes Co. under the trade name WANNATE®.

In one aspect, the polyisocyanate is added to the first admixture at different ratios such as, for example, about 1:1, about 1:2, about 1:3, about 1:4, about 1:5, about 1:6, about 1:7, or about 1:8 with respect to the total emulsion volume, or any range thereof (e.g., 1:1 to 1:8, 1:3 to 1:5, etc.). In this aspect, polymerization reactions can be carried out. Different reaction times (e.g., 8 minutes, 10 minutes, 12 minutes, 15 minutes, or 20 minutes) and stirring speeds (e.g., 500 rpm, 600 rpm, 700 rpm, 800 rpm, 900 rpm, or 1000 rpm) can be evaluated to determine the optimum reaction time and stirring speed. In one aspect, the first admixture is admixed with the polyisocyanate for 10 minutes at 500 rpm. In another aspect, the reaction is conducted at room temperature.

In some aspects, the anti-microbial and UV-protective extracts are added to the second admixture containing the isocyanate, or, alternatively, are already present in the isocyanate solution when it is incorporated into the first admixture.

Upon admixing the components in the first admixture with the polyisocyanate, isocyanate-reactive functional groups present on the polyactive carbohydrate and/or the carbo sugar and/or the natural oil polyol and/or the anti-microbial and UV-protective extracts react with the isocyanate groups on the polyisocyanate to produce a polyurethane. Here, a polymer composed of organic residues joined by urethane linkages is produced. Although the components in the first admixture include hydroxyl groups, other components may be present that can include other isocyanate-reactive functional groups such as amine groups, thiol groups, or other nucleophilic groups capable of reacting with isocyanate groups.

The amount of the carbo sugar, if used, in the final biofoam product can vary. In one aspect, the amount of carbo sugar present in the biofoam is from 0.005% to 0.1% by weight of the biofoam. In another aspect, the amount of carbo sugar present in the biofoam is 0.005%, 0.01%, 0.02%, 0.03%, 0.04%, 0.05%, 0.06%, 0.07%, 0.08%, 0.09%, or 0.1% by weight of the biofoam, where any value can be a lower and/or upper endpoint of a range (e.g., 0.01% to 0.05%). When used to prepare the biofoams, the carbo sugar can be prepared as a stock solution. For example, the carbo sugar in powder form (0.05 g, 0.1 g, 0.2 g, 0.3 g, 0.4 g, 0.5 g, 0.6 g, 0.7 g, 0.8 g, 0.9 g, or 1 g) can be added to water (100 mL to 1 L) to produce a stock solution. The pH of the stock solution can be adjusted with standard buffer solutions. In one aspect, the pH of the carbo sugar stock solution is from 1 to 5, 1.5 to 4, or 2 to 3.

The amount of the polyactive carbohydrate present in the final biofoam product can vary. In one aspect, the amount of polyactive carbohydrate present in the biofoam is from 0.005% to 0.1% by weight of the biofoam. In another aspect, the amount of polyactive carbohydrate present in the biofoam is about 0.005%, 0.01%, 0.02%, 0.03%, 0.04%, 0.05%, 0.06%, 0.07%, 0.08%, 0.09%, or 0.1% by weight of the biofoam, where any value can be a lower or upper endpoint of a range (e.g., 0.01% to 0.05%). When used to prepare the biofoams, the polyactive carbohydrate can be prepared as a stock solution. For example, the polyactive carbohydrate in powder form (0.05, 0.1, 0.2, 0.3, 0.4, 0.5, 0.6, 0.7, 0.8, 0.9, or 1 g) can be added to water (100 mL to 1 L) to produce a stock solution. In some aspects, the anti-microbial and UV-protective extracts are also admixed with this stock solution. The pH of the stock solution can be adjusted as necessary.

The selection and amounts of reactants as well as processing conditions will determine the physical state of the biofoams. In one aspect, when the polyisocyanate is admixed with the first admixture, a solid biofoam is produced. The polyurethane compositions produced herein can be poured into a mold of any desired shape. If necessary, the mold containing the polyurethane composition can be placed in an oven to remove residual solvent and produce the final biofoam.

In other aspects, one or more blowing agents can be incorporated into the polyurethane compositions to produce the biofoams. A blowing agent can be physical or chemical in nature. A “physical blowing agent” is a gas or low boiling point liquid which expands due to heat generated by the polyurethane-forming reaction, thus forming bubbles and creating foam. A “chemical blowing agent” is a compound or substance that reacts to form a gas. In one aspect, the blowing agent is a physical blowing agent. Physical blowing agents include compounds such as, for example, hydrofluorocarbons (HFCs), hydrocarbons (HCs), hydrofluoroolefins, liquid CO₂, and other low boiling point liquids. In one aspect, the physical blowing agent is HFC-134a (1,1,1,2-tetrafluoroethane), HFC-245fa (pentafluoropropane), HFC-365mfc (1,1,1,3,3-pentafluorobutane), HFC-152a (1,1-difluoroethane), formic acid, methyl formate, HFO-1234ze (1,3,3,3-tetrafluoropropene), cyclopentane, n-pentane, iso-pentane, iso-butane, acetone, dichloromethane, or a mixture thereof. In another aspect, the blowing agent is a chemical blowing agent. In one aspect, the chemical blowing agent is carbon dioxide produced by the reaction of isocyanate groups with water. In a further aspect, both chemical and physical blowing agents can be used.

In other aspects, the biofoams include additional additives not already described above such as, for example, flame retardants, color additives, release agents, biocides, other additives, or a combination thereof. The additional components can be admixed with a dispersion or emulsion of polyurethane composition in order to incorporate the additives throughout the biofoam. In the alternative, the additives can be applied to the surface of the solid biofoam.

In another aspect, after the preparation of the biofoam, the biofoam can contain residual solvent (e.g., water). In certain aspects, it is desirable to remove all or substantially all (e.g., greater than 70%, greater than 80%, greater than 90%, greater than 95%, greater than 99%, or 100%) of the solvent in the biofoam. In one aspect, drying of the biofoams can be accomplished in an oven at about 20° C., 30° C., 40° C., 50° C., 60° C., or about 70° C. In one aspect, the biofoams are dried in an oven at 50° C. In a further aspect, the biofoams can be dried for from about 0.5 to ab08t 100 hours, or for about 72 hours. In one aspect, removal of water from biofoams is assessed by periodically removing the biofoams from the oven and weighing them. When the biofoams have the same weight at, for example, at least 2 or 3 successive weighings separated by several hours, the biofoams can be considered to be dry and can be removed from the oven.

The biofoams produced herein have several beneficial properties. In one aspect, the biofoams are resistant to discoloration. In another aspect, discoloration of the biofoams can be assessed by exposing the biofoams to an agent known to cause stains. In a further aspect, the agent known to cause stains is, for example, tea, coffee, or red wine. In one aspect, the biofoams can be submersed in coffee for a period of up to about 24 hours. In this aspect, after 24 hours, the biofoams are removed from the coffee and rinsed with water. Discoloration can then be qualitatively assessed as, for example, weak, medium, or strong.

In another aspect, the biofoams are resistant to acid degradation. For example, the biofoam can be assessed by placing a piece of the foam in an aqueous solution of an acid for 24 or 48 hours. In a further aspect, the acid is present at a 0.1N concentration. In another aspect, the acid is an organic acid such as, for example, acetic acid or formic acid. In an alternative aspect, the acid is an inorganic acid such as, for example, nitric acid, hydrochloric acid, phosphoric acid, or sulfuric acid. Resistance to mixtures of acids can also be tested. In a further aspect, photographs of the foam before and after exposure to acid can be compared to qualitatively assess acid resistance. In another aspect, the foam can be weighed before and after acid exposure to assess whether material has been lost.

In one aspect, it is desirable to know the maximum temperature to which the biofoams can be exposed without decomposition. This is known as temperature resistance. In one aspect, decomposition due to heat exposure can be assessed by placing a piece of the foam in an oven at a temperature of from about 50° C. to about 120° C. In a further aspect, temperature resistance is assessed at about 50° C., at about 80° C., or at about 120° C. In certain aspects, pieces of biofoam can be placed in an oven and the internal temperatures of the biofoam pieces can be measured periodically with, for example, a thermometer or a thermocouple. In a further aspect, temperature resistance can be measured every 10 minutes for up to one hour. In one aspect, the biofoam samples can be weighed prior to assessing temperature resistance, and can be weighed periodically to evaluate the level of decomposition. In this aspect, samples can be weighed every 10 minutes for up to one hour, at about the same time the internal temperature of the biofoam pieces is being measured, with weight loss indicating that decomposition has occurred. In an additional aspect, temperature resistance can be qualitatively assessed by, for example, visually noting any discoloration of the biofoam samples that occurs subsequently to heat treatment. In one aspect, if a sample exhibits less than about 20% weight loss, or less than about 10% weight loss, after exposure to a particular temperature, the sample can be said to be temperature resistant. In another aspect, if a sample does not become visibly discolored after exposure to a particular temperature, the sample can be said to be temperature resistant.

In one aspect, it is desirable to assess the biofoams for recovery from deformation. In this aspect, pressure can be applied to the biofoams, causing deformation. Also in this aspect, when pressure is removed from the biofoams, the biofoams can return to their original shapes and/or sizes. In certain aspects, from about 0.5 bars to about 1 bar of pressure are applied. In other aspects, the time required for the biofoams to recover from deformation is measured. In one aspect, the biofoams take up to about 5 seconds to recover from deformation. In another aspect, the biofoams take from about 1 second to about 3 seconds to recover from deformation.

The polyurethane compositions described herein can be used to produce biofoams that have numerous applications. The term “biofoam” as used herein is any substance formed when pockets of gas have been trapped in a solid or liquid. In one aspect, the biofoams produced herein can exist as an emulsion or dispersion at room temperature. In other aspects, the biofoams produced herein are solid materials at room temperature.

In one aspect, provided herein are articles composed of or including the biofoams described herein. The biofoams produced herein can be used in any application where soft, synthetic polyurethane foams are used. For example, the biofoams can be used in upholstery such as cushions, pillows, furniture, or mattresses, including in automobiles, trains, watercraft and boats, and aircraft. In another aspect, the biofoams can be used to produce equipment for exercise or physical therapy including, for example, yoga mats and other floor mats, padding or upholstery for weight machines and seating for stationary and street bicycles, foam balls for physical therapy, comfort grips for handles for weights, kettlebells, bicycles, and the like, helmet padding and other personal protective equipment, and similar applications. In still another aspect, the biofoams can be used in the construction industry such as for insulation and carpet padding or carpet underlay materials. In another aspect, the biofoams can be used to create packaging materials including anti-static cushioning, case inserts, pads for vibration control, camping pads, and the like.

In another aspect, the biofoams disclosed herein can be used in the medical industry. In one aspect, the biofoams can be used where it is desirable to reduce or minimize blunt force or trauma to a subject. For example, the polyurethane composition can be injected between the skin of the subject and a cast to produce a biofoam that can further prevent any applied force to the broken bone of the subject. In certain aspects, the polyurethane composition can include anti-microbial agents in order to prevent odor.

In one aspect, the biofoams disclosed herein can be used to manufacture disposable cups and other packaging and containers intended for holding, transporting, and/or storing food and beverage items. In one aspect, the cups are impervious to water for a period of time ranging from a few hours to six months. In another aspect, the cups are impervious to hot and cold temperatures ranging from 10° C. to 65° C. In still another aspect, the cups are made from 70% to 100% natural and/or organic ingredients and are biodegradable. In yet another aspect, the food or beverage containers have anti-microbial properties that may delay or inhibit spoilage of food and beverages stored therein.

In one aspect, the biofoams can be formed using molds or 3D printers into construction materials, materials such as seat padding or upholstery foam used in the airplane and automobile industries, as insulation against freezing or heat, or as noise damping or acoustic materials.

### VII. Additional Applications of the UV-Protective and Anti-Microbial Extracts

### A. Adhesives

In other aspects, the polyurethane compositions described herein can be used as adhesives. For example, the polyurethane composition can be in a sufficient amount of solvent so that it can readily be applied to the surface of a substrate (e.g., spray coating, dipping, brushing). Upon removal of the solvent, a biofoam is produced, which results in the formation of a strong bond between two substrates. In other aspects, the polyurethane compositions can be used to seal cracks and holes. Here, the polyurethane composition is sprayed in a crack or hole and then forms a biofoam.

In another aspect, the adhesives can adhere plastic to plastic, metal to metal, wood to wood, plastic to metal, plastic to wood, and/or metal to wood. In one aspect, the adhesives are biodegradable and are designed break down over time. In an alternative aspect, under appropriate environmental conditions, the adhesives can last for months or even years.

### B. Enhanced Plant Growth

In one aspect, the biofoams described herein can be granulated and added to soil in order to enhance plant growth. For example, when the granules are mixed with soil and seeds are planted therein, seedlings exposed to granules in the soil are more robust than seedlings planted in soil without granules (see Examples). In one aspect, the seedlings are taller and/or have broader leaves. In another aspect, the seedlings produce more chlorophyll and/or other pigments. In still another aspect, the granules are biodegradable and break down over time so they do not contaminate the soil with which they are mixed. In one aspect, the seeds planted can be corn, beans, tomatoes, peppers, peas, squash, cucumber, eggplant, radish, beet, turnip, melon, or any other fruit or vegetable commonly grown from seed. In another aspect, the seeds can be ornamental plants or herbs such as, for example, cilantro/coriander, thyme, oregano, basil, rosemary, savory, marjoram, chives, parsley, sage, dill, or other commonly used culinary or medicinal herbs.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperatures, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions (e.g., component concentrations, desired solvents, solvent mixtures, temperatures, pressures, and other reaction ranges and conditions can be used to optimize the product purity and yield obtained from the desired process. Only reasonable and routine experimentation will be required to optimize such processes and conditions.

### Example 1: Preparation of DNA Construct for Production of Anti-Microbial and UV-Protective Extracts

### A. Devices for Production of Anti-Microbial and UV-Protective Extracts

The DNA construct was composed of the genetic components described herein and assembled in plasmid vectors (e.g., pYES2, purchased from Invitrogen). Sequences of genes and/or proteins with desired properties were identified in GenBank; these include a gene that expresses zinc-related protein/oxidase, a gene that expresses silicatein, a gene that expresses silaffin, and a gene that expresses alcohol dehydrogenase II. The sequences were synthesized by CloneTex Systems, Inc. (Austin, TX). Other genetic parts were also obtained for inclusion in the DNA constructs including, for example, promoter genes (e.g., GAL1 promoter), reporter genes (e.g., yellow fluorescent reporter protein), and terminator sequences (e.g., CYC1 terminator). These genetic parts included restriction sites for ease of insertion into plasmid vectors.

The cloning of the DNA construct into the biological devices was performed as follows. Sequences of individual genes were amplified by polymerase chain reaction using primers that incorporated restriction sites at their 5′ ends to facilitate construction of the full sequence to be inserted into the plasmid. Genes were then ligated using standard protocols to form an insert. The plasmid was then digested with restriction enzymes (AscI and KpnI) according to directions and using reagents provided by the enzymes' supplier (New England Biolabs).

From 5′ to 3′, the construct includes (a) a gene that expresses zinc-related protein/oxidase, (b) a CYC1 terminator, (c) a GAL1 promoter, (d) a gene that expresses silicatein, (e) a CYC1 terminator, (f) a GAL1 promoter, (g) a gene that expresses silaffin, (h) a CYC1 terminator, (i) a GAL1 promoter, (j) a gene that expresses alcohol dehydrogenase II, (k) a CYC1 terminator, and (1) a yellow fluorescent reporter protein assembled in pYES2 plasmid (SEQ ID NO. 16) (FIGS. 1A and 1B).

Also prepared was a second construct. From 5′ to 3′, the second construct includes (a) a gene that expresses lipase, (b) a CYC1 terminator, (c) a GAL1 promoter (d) a gene that expresses zinc-related protein/oxidase, (e) a CYC1 terminator, (f) a GAL1 promoter, (g) a gene that expresses silicatein, (h) a CYC1 terminator, (i) a GAL1 promoter, (j) a gene that expresses silaffin, (k) a CYC1 terminator, (1) a GAL1 promoter, (m) a gene that expresses alcohol dehydrogenase II, (n) a CYC1 terminator, and (o) a yellow fluorescent reporter protein assembled in pYES2 plasmid (SEQ ID NO. 17) (FIGS. 2A and 2B).

PCR was used to enhance DNA concentration using a Mastercycler Personal 5332 ThermoCycler (Eppendorf North America) with specific sequence primers and the standard method for amplification (Sambrook, J., E. F. Fritsch, and T. Maniatis, 1989, Molecular Cloning: A Laboratory Manual, 2^{nd} ed., Vol. 1, Cold Spring Harbor Laboratory Press: Cold Spring Harbor, NY). PCR amplified pieces of all fragments were combined using homologous recombination technology (Gibson Assembly). The complete insert, containing restriction sites on each end, was then ligated into the plasmid. Successful construction of the insert and ligation of the insert into the plasmid were confirmed by gel electrophoresis and DNA sequencing.

Digestion and ligation were used to ensure assembly of synthesized DNA parts using restriction enzymes and reagents (PCR reagents and enzymes were purchased from New England Biolabs). DNA was quantified using a Nano Vue spectrophotometer (GE Life Sciences) and a standard UV/Visible spectrophotometer using the ratio of absorbances at 260 nm and 280 nm. In order to verify final ligations, DNA was visualized and purified via electrophoresis using a Thermo EC-150 power supply.

The DNA construct was made with gene parts fundamental for expression of sequences such as, for example, native and constitutive promoters, reporter genes, and transcriptional terminators or stops. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the method described below.

### B. Devices for Production of a Polyactive Carbohydrate

DNA constructs for producing polyactive carbohydrates were constructed in the same manner as for DNA constructs producing UV-protective and anti-microbial extracts as described above. Plasmids used for these constructs included pYES2, pBSK, and pETDuet-1. Sequences of genes and/or proteins with desired properties were identified in GenBank; these included a gene that expresses lipase, a gene that expresses chitin synthase, a gene that expresses chitosanase, and a gene that expresses chitin deacetylase. These sequences were synthesized by CloneTex Systems, Inc. (Austin, TX). Other genetic parts were also obtained for inclusion in the DNA constructs including, for example, promoter genes (e.g., GAL1 promoter), reporter genes (e.g., yellow fluorescent reporter protein), and terminator sequences (e.g., CYC1 terminator). These genetic parts included restriction sites for ease of insertion into plasmid vectors. Lipase was included in some constructs and was functional at any position in the construct. However, a position 5′ of the gene for expressing chitin synthase was preferable when the lipase gene was included.

From 5′ to 3′, one version of the construct includes (a) a gene that expresses chitin synthase, (b) a CYC1 terminator, (c) a GAL1 promoter, (d) a gene that expresses chitosanase, (e) a CYC1 terminator, (f) a GAL1 promoter, (g) a gene that expresses chitin deacetylase, (h) a CYC1 terminator, (i) a GAL1 promoter, and (j) a yellow fluorescent reporter protein (SEQ ID NO. 18) (FIGS. 3A and 3B).

From 5′ to 3′, a second version of the construct includes (a) a gene that expresses lipase, (b) a CYC1 terminator, (c) a GAL1 promoter, (d) a gene that expresses chitin synthase, (e) a CYC1 terminator, (f) a GAL1 promoter, (g) a gene that expresses chitosanase, (h) a CYC1 terminator, (i) a GAL1 promoter, (j) a gene that expresses chitin deacetylase, (k) a CYC1 terminator, (l) a GAL1 promoter, and (m) a yellow fluorescent reporter protein (SEQ ID NO. 19) (FIGS. 4A and 4B).

Successful construction of the constructs was assessed as described previously for the UV-protective and anti-microbial constructs.

### C. Devices for Production of a Carbo Sugar

DNA constructs for producing carbo sugars were constructed in the same manner as for DNA constructs producing UV-protective and anti-microbial extracts as described above. The plasmids used for these constructs was pYES2. Sequences of genes and/or proteins with desired properties were identified in GenBank; these included a gene that expresses lipase, a gene that expresses galactomannan galactosyltransferase, and a gene that expresses cellulose synthase. These sequences were synthesized by CloneTex Systems, Inc. (Austin, TX). Other genetic parts were also obtained for inclusion in the DNA constructs including, for example, promoter genes (e.g., GAL1 promoter), reporter genes (e.g., yellow fluorescent reporter protein), and terminator sequences (e.g., CYC1 terminator). These genetic parts included restriction sites for ease of insertion into plasmid vectors. Lipase was included in some constructs and was functional at any position in the construct. However, a position 5′ of the gene for expressing cellulose synthase was preferable when the lipase gene was included.

From 5′ to 3′, the construct includes (a) a gene that expresses cellulose synthase, (b) a CYC1 terminator, (c) a GAL1 promoter, (d) a gene that expresses galactomannan galactosyltransferase, (e) a CYC1 terminator, and (f) a yellow fluorescent reporter protein (SEQ ID NO. 20) (FIGS. 5A-5D).

From 5′ to 3′, the construct includes (a) a gene that expresses lipase, (b) a CYC1 terminator, (c) a GAL1 promoter, (d) a gene that expresses cellulose synthase, (e) a CYC1 terminator, (f) a GAL1 promoter, (g) a gene that expresses galactomannan galactosyltransferase, (h) a CYC1 terminator, and (i) a yellow fluorescent reporter protein (SEQ ID NO. 21) (FIGS. 6A-6B).

From 5′ to 3′, the construct includes (a) a gene that expresses lipase, (b) a T7 promoter, (c) a LAC operon, (d) a riboswitch, (e) a gene that expresses cellulose synthase, (f) a riboswitch, and (g) a gene that expresses galactomannan galactosyltransferase (SEQ ID NO. 22) (FIGS. 7A-7B).

Successful construction of the constructs was assessed as described previously for the UV-protective and anti-microbial constructs.

### Example 2: Selection of Microorganisms

The anti-microbial and UV-protective extract was produced using transfected yeasts (Saccharomyces cerevisiae, ATCC® 200892™) and/or bacteria (Escherichia coli, ONESHOT® Top10 competent cells from Life Technologies™, BL21 (DE3) E. coli from Novagen, Inc., or DH5α™ E. coli from Thermo Fisher Scientific).

### Example 3: Development of Competent Yeast Cells

Yeast cells were made competent by subjecting them to an electrochemical process adapted from Gietz and Schiestl (Nature Protocols, 2007, 2:35-37). Briefly, a single yeast colony was inoculated into 100 mL YPD (yeast extract peptone dextrose) growth media. Yeast was grown overnight on a shaker at 30° C. to OD₆₀₀=1.0. (Acceptable results were obtained with OD₆₀₀ values ranging from 0.6 to 1.8.) Cells were centrifuged at 2000 rpm in a tabletop centrifuge and resuspended in 10 mL TEL buffer (10 mM Tris-HCl, 1 mM EDTA, 0.1 M LiAc, pH=7.5) and shaken vigorously overnight at room temperature. Cells were again centrifuged and resuspended in 1 mL TEL buffer. Cells prepared in this manner could be stored in the refrigerator for up to one month.

In other experiments, competent yeast cells (strain INVSc1) were purchased from Invitrogen, Inc. and prepared and transformed using a kit purchased from Sigma-Aldrich, Inc.

### Example 4: Transformation of Yeast Cells to Produce Anti-Microbial and UV-Protective Device, Polyactive Carbohydrates, and Carbo Sugars

A clone with 100% accuracy in the desired DNA sequence was selected for further processing. The selected clone was used to obtain a high concentration of the plasmid construct at a mid-scale plasmid purification level. Cells containing the plasmid were selected on a synthetic complete (SC) dropout plate deficient in uracil. A well-isolated clone was chosen from the SC plate and preserved in YPD medium containing 15% glycerol for storage at −80° C.

Competent cells were stored in the freezer until needed. Cells were thawed on ice and 100 μL of competent cells in TEL buffer were placed in a sterile 1.5 mL microcentrifuge tube. To this was added 5 μL of a 10 mg/mL solution of salmon sperm DNA (carrier DNA). Transforming DNA was added in various amounts. From 1 to 5 μg was sufficient for plasmids from commercial sources, but more DNA was required when transforming yeast with artificial DNA constructs. 10 μL of the DNA device were added to the microcentrifuge tube containing the competent yeast cells and the contents of the tube were mixed. The DNA-yeast suspension was incubated for 30 min at room temperature.

A PLATE solution (consisting of 40% PEG-3350 in 1×TEL buffer) was prepared. 0.7 mL of PLATE solution was added to the DNA-yeast suspension and the contents were mixed thoroughly and incubated for 1 h at room temperature. The mixture was placed in an electromagnetic chamber for 30 minutes. Cells were then heated at 42° C. for 5-10 minutes and 250 μL aliquots were plated on yeast malt agar to which selective growth compounds had been added. Plates were incubated overnight at 30° C.

DNA expression and effectiveness of transformation were determined by fluorescence of the transformed cells expressed in fluorescence units (FSUs) using a 20/20 Luminometer (Promega) according to a protocol provided by the manufacturer. Plasmid DNA extraction, purification, PCR, and gel electrophoresis were also used to confirm transformation. Different transformed devices were obtained. Different types of fluorescent reporter proteins were used (e.g., yellow, red, green, and cyan) for all transformed cells and/or constructs. However, the yellow fluorescent protein was preferred. When no fluorescent reporter protein was assembled, no fluorescence was observed.

S. cerevisiae cells were subject to transformation with the modified pYES2 plasmid as described above. Transformed yeast cells were incubated for 30 min at 28-30° C. Colonies of transformed yeast cells were selected, their DNA isolated and subjected to PCR amplification. Two control treatments were also carried out: (1) a negative control involving competent yeast and nuclease free water instead of a plasmid and (2) a positive control involving competent yeast with unmodified pYES2 plasmid.

Alternatively, the pETDuet-1 plasmid-based device was transformed into DH5α and BL21 (DE3) E. coli using a standard heat shock protocol. Four clones were selected from a transformed plate and processed for full-length DNA sequencing. A clone with 100% DNA sequence accuracy was selected for further processing and was used to obtain a high concentration of plasmid construct at a mid-scale plasmid purification level.

### Example 5: Induction and Growth of Device

The following procedure was used to induce production of the extract from the UV-protective and anti-microbial devices described previously. A small sample of yeast device transformed with the construct in FIGS. 1A-1B (SEQ ID NO. 16) was grown in 3-5 mL of yeast malt overnight at 30° C.

1 mL of the device grown overnight was added to 1 L of yeast malt containing at least 2% raffinose sugar and incubated at 30° C. for 2-4 hours until device growth reaches 0.6-0.8 optical density as measured by UV-Vis spectrophotometry. Galactose sugar at 1% based on a 1 L culture is added to the previously described culture, then incubated for at least 48 hours at 30° C.

### Example 6: Isolation of Anti-Microbial and UV-Protective Extracts

After 48 hours of culture as described in Example 5, the culture of the UV-protective and anti-microbial device was treated with lyticase (240 μL/L) for 24 hours. The culture was then centrifuged at 9,000 rpm for 15 minutes to obtain a pellet. The supernatant is not discarded. The pellet was resuspended in distilled water at a level of approximately 1 g pellet per 100 mL of water. This mixture was subjected to sonication for 2 minutes, using a 30 seconds on/15 seconds off program at 60% of wavelength amplitude (QSONICA Sonicator, Newtown, CT). This procedure was repeated twice.

The supernatant from the centrifugation is again centrifuged to remove dead cells and/or debris. The centrifuged supernatant is then filtered through a 0.45 μm pore filter. The filtrate contains antifungal compounds and metabolites and can be used for antifungal treatment.

### Example 7: Antifungal Testing Using the Biological Devices

Antifungal capabilities of the extract produced in Example 6 were evaluated using Fusarium graminearum (ATCC 15624). The minimal fungal concentration in culture was 10⁴ cells/mL. Fungal cultures were mixed with the extract produced above (10% of either extract based on total solution volume).

Samples of the above mixture were obtained at 0, 30, and 60 minutes, as well as 24 hours, 48 hours, and 72 hours and fungal growth was determined. At each sampling time, 500 μL aliquots of the fungal culture were taken into PDA agar plates to confirm whether the fungal cells obtained were alive or dead. Antifungal effectiveness was determined by counting fungal colonies; photographic records were also collected. A control experiment with a fungal culture extract and no biological device was also performed.

The extract inhibited growth of the fungus Fusarium graminearum after 24 hours of incubation, as compared to control (i.e., fungal culture without treatment with extract), which showed full growth.

### Example 8: Production of Soft Biofoams

The following procedures were used to construct soft biofoams from extracts of the devices disclosed herein. These biofoams are 90% natural and 10% synthetic.

### A. Polyactive Carbohydrate Extract

1. Yeast transformed with the device depicted in FIGS. 3A and 3B (SEQ ID NO. 18) was fermented in yeast malt medium with 2% raffinose and 1 mg/ml of glucosamine and induced with galactose at 30° C. for 72 hours.
2. The culture medium was centrifuged at 9,000 rpm for 15 minutes to pelletize the culture.
3. The pellet was resuspended at 1 g/50 mL in sterile deionized water.
4. The resuspension was sonicated for two minutes and 30 seconds using a 30 seconds on/15 seconds off protocol.
5. The sonicated mixture was centrifuged at 9,000 rpm for 15 minutes.
6. The supernatant was filtered with a 0.45 μm filter.

### B. Anti-Microbial and UV-Protective Extract

1. Yeast transformed with the device depicted in FIGS. 1A and 1B (SEQ ID NO. 16) was fermented in yeast malt medium with 2% raffinose and induced with galactose at 30° C. for 72 hours.
2. The culture medium was centrifuged at 9,000 rpm for 15 minutes to pelletize the culture.
3. The pellet was resuspended at 1 g/50 mL in sterile deionized water.
4. The resuspension was sonicated 3 times for 2 minutes and 30 seconds each time.
5. The sonicated mixture was centrifuged at 9,000 rpm for 15 minutes.
6. The supernatant was filtered with a 0.45 μm filter.

### C. Construction of a Soft Biofoam Containing a Polyactive Carbohydrate Extract and an Anti-Microbial and UV-Protective Extract

At room temperature, 19 mL of polyactive carbohydrate extract as described above and 19 mL of anti-microbial/UV-protective extract (optical density 2.4) were mixed with 2 mL of polysorbate 80 and stirred for 3 minutes. 50 mL of castor oil were added and the mixture was stirred for an additional 10 minutes.

Separately, also at room temperature, 5 mL of each extract were mixed with 0.5 g of bentonite and vortexed for 5 minutes. This second mixture was added to the above mixture and stirred for an additional 10 minutes.

20 mL of isocyanate (MDI; Geos Quimica S.A.S.) were added to the above mixture over the course of 7 minutes at room temperature; this represented a ratio of approximately 5:1 polyol:isocyanate. Pressurized air (5-15 psi) was injected during the last 2 minutes of this process if required for continued mixing, although in some cases air was not required.

The mixture was allowed to dry overnight at room temperature, then transferred to a mold to complete the final formation of the biofoam. Molds of various shapes and sizes were used, depending on the intended use of the final biofoam product.

After formation of the biofoams, they were removed from the molds and the biofoam objects were transferred to an oven to complete drying at 30-40° C. for 30-60 minutes.

### D. Physical Characteristics of the Soft Biofoams Containing a Polyactive Carbohydrate and Anti-Microbial UV-Protective Extracts

The soft biofoams once dried had a soft texture with memory (i.e., would return to the original shape after deformation). For a 7 cm³ cube, the mass of the biofoam was approximately 63 g. These biofoams also exhibited antimicrobial properties.

### Example 9: Production of Hard Biofoams

### A. Procedure for Making Biofoams Containing a Polyactive Carbohydrate and Anti-Microbial UV-Protective Extracts

The following procedures were used to construct hard biofoams from extracts of the devices disclosed herein. These biofoams are 90% natural and 10% synthetic and the polyactive carbohydrate and anti-microbial/UV-protective extracts were prepared as described in Example 8. In some experiments, carbo sugar extracts were used in place of or in addition to the polyactive carbohydrate extracts.

At room temperature, 24.5 mL of each respective extract were mixed together with 1 mL of polysorbate 80 for 3 minutes. 40 mL of castor oil were added and the mixture was stirred for an additional 10 minutes.

Separately, and also at room temperature, 5 mL of each respective extract were mixed together with 0.5 g of bentonite and vortexed for 5 minutes. This mixture was added to the above solution and stirred for 10 minutes.

20 mL of isocyanate (MDI; Geos Quimica S.A.S.) were added to the above mixture over the course of 7 minutes at room temperature; this represented a ratio of approximately 5:1 polyol:isocyanate. Pressurized air (5-15 psi) was injected during the last 2 minutes of this process if required for continued mixing, although in some cases air was not required.

The mixture was allowed to dry overnight at room temperature, then transferred to a mold to complete the final formation of the biofoam. Molds of various shapes and sizes were used, depending on the intended use of the final biofoam product.

After formation of the biofoams, they were removed from the molds and the biofoam objects were transferred to an oven to complete drying at 30-40° C. for 30-60 minutes.

### B. Physical Characteristics of the Hard Biofoams Containing a Polyactive Carbohydrate and Anti-Microbial UV-Protective Extracts

The hard biofoams once dried had a hard texture with no memory (i.e., would not return to the original shape after deformation). For a 7 cm³ cube, the mass of the biofoam was approximately 55 g. These biofoams also exhibited antimicrobial properties.

### C. Chemical and Mechanical Properties of Hard Biofoams

The hard biofoams described herein were subjected to various physical and chemical tests. The hard biofoams could be placed into cup-shaped molds and formed into cups.

A piece of hard biofoam was immersed in water for six months and remained floating in the water at the end of that time period. This indicated that the hard biofoam was impermeable to water. A piece of hard biofoam was also immersed in 1% acetic acid for six months. It did not degrade, but remained floating in the solution. This indicated that the hard biofoam was resistant to mild acid.

A hammer was used to strike a block of hard biofoam. The hard biofoam did not break or splinter, indicating resistance to physical impact.

### Example 10: Preparation of Biodegradable Cups from Polyactive Carbohydrate Extracts and Anti-Microbial/UV-Protective Extracts

The following procedures were used to prepare and optimize the composition of biodegradable cups from extracts of the devices disclosed herein. These cups range from 79-97% natural and 3-21% synthetic and the polyactive carbohydrate and anti-microbial/UV-protective extracts were prepared as described in Example 8. In some experiments, device cultures were allowed to grow for up to 190 hours, but 72 hours was generally sufficient.

At room temperature, a total of 49 mL of extracts from the devices disclosed herein (usually equal volumes of the polyactive carbohydrate extract and the anti-microbial/UV-protective extract produced in Example 8) were added to the surfactant and stirred for 3 minutes. Castor oil was added and stirring was carried out for an additional 10 minutes. Separately, a total of 10 mL of extracts from the devices disclosed herein (again, usually equal volumes) were added to bentonite and the mixture was vortexed for 5 minutes. This second mixture was added to the first and stirring took place for an additional 10 minutes.

Following this, isocyanate (typically MDI from Geos Quimica S.A.S.) was added in a ratio of 4:1 polyol:isocyanate (typically 25 mL isocyanate) and stirred for 10 minutes at room temperature. This mixture was allowed to dry for 3 minutes at room temperature and excess water was removed from the mixture.

The mixture was then transferred to a 60 cc Teflon mold to complete the final formation of the cup. Molds of various sizes can be used depending on the cup size and shape desired.

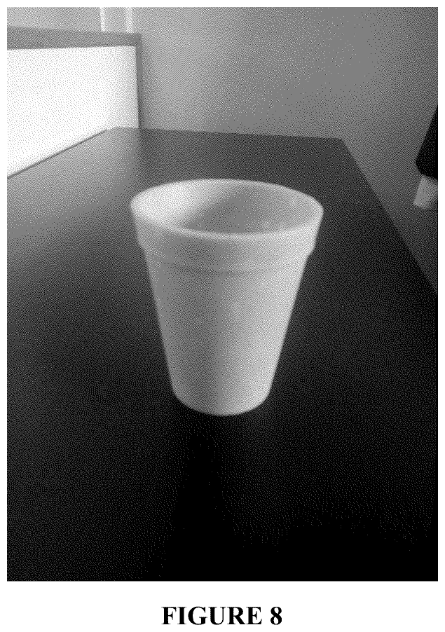

After 24 hours, the cup was removed from the Teflon mold and transferred to an oven to dry for 30-60 minutes at 30-40° C. (FIG. 8).

Texture, size, and weight of cups varied depending upon the composition. All cups constructed were impermeable to water when tested with water of different temperatures ranging from 10-65° C. A typical cup from a 60 cc mold weighed from 30-35 g and had a hard, smooth texture. Different cup compositions are provided in Table 11.

TABLE 11
Composition of Sample Biofoam Cups

| Component or Property | Cup A | Cup B | Cup C | Cup D |
|---|---|---|---|---|
| % Natural | 78.6 | 86.54 | 94.87 | 96.89 |
| % Synthetic | 21.4 | 13.45 | 5.12 | 3.13 |
| Polyactive carbohydrate | 29.5 | 60 | 70 | 94 |
| extract (mL) | | | | |
| Anti-microbial/UV- | 29.5 | 60 | 70 | 70 |
| protective extract (mL) | | | | |
| Castor oil (mL) | 40 | 50 | 20 | 10 |
| Beeswax cream | 0 | 20 | 20 | 10 |
| (5%, mL) | | | | |
| Polysorbate 80 (mL) | 1 | 2 | 4 | 2 |
| Isocyanate (mL) | 25 | 30 | 10 | 6 |
| Bentonite (g) | 0.5 | 1 | 1 | 1 |
| NaH (drops) | 0 | 3 | 3 | 3 |

### Example 11: Preparation of Biodegradable Cups from Polyactive Carbohydrate Extract, Carbosugar Extract, and Anti-Microbial/UV-Protective Extract

The following procedure was used to produce a cup. At room temperature, the polyactive carbohydrate extract (65 mL, concentration of 2.5 OD produce from yeast cells transformed with construct in Example 1) and the anti-microbial/UV-protective extract (35 mL, concentration of 2.5 OD produced in Example 8) were added to the surfactant (TWEEN 80, 2 mL) and stirred for 3 minutes. Castor oil (15 mL) was added and stirring was carried out for an additional 5 minutes. Separately, 5 mL each of the polyactive carbohydrate extracts and the anti-microbial/UV-protective extract were added to bentonite and the mixture was vortexed for 5 minutes. This second mixture was added to the mixture above and stirring took place for an additional 10 minutes.

In a separate mixture, polyisocyanate (Geos Quimica S.A.S—Isocyanate for rigid—MDI) (6 mL), beeswax cream (10 ml at 5 wt %) and carbo sugar extract produce from yeast cells transformed with construct in Example 1 (40 ml) were mixed for 9 minutes at room temperature (25°−28° C.). This mixture was admixed with the mixture above and stirred for 10 minutes at room temperature. This mixture was allowed to dry for 3 minutes at room temperature and excess water was removed from the mixture.

The mixture was then transferred to a 60 cc Teflon mold to complete the final formation of the cup. Molds of various sizes can be used depending on the cup size and shape desired.

After 24 hours, the cup was removed from the Teflon mold and transferred to an oven to dry for 30-60 minutes at 30-40° C.

### Example 12: Determination of Polyactive Carbohydrate Concentration in Culture Extracts

Concentrations of polyactive carbohydrate produced in Example 8 used to produce the cups and in subsequent experiments were determined as follows. Samples from device cultures were obtained and centrifuged at 9,000 rpm for 15 minutes at 15° C. and a pellet was obtained. The pellet was sonicated for two minutes and 30 seconds using a 30 seconds on/15 seconds off protocol. The resulting mixture was centrifuged at 9,000 rpm for 15 minutes and the supernatant was filtered using 0.45 μm filters to obtain an extract.

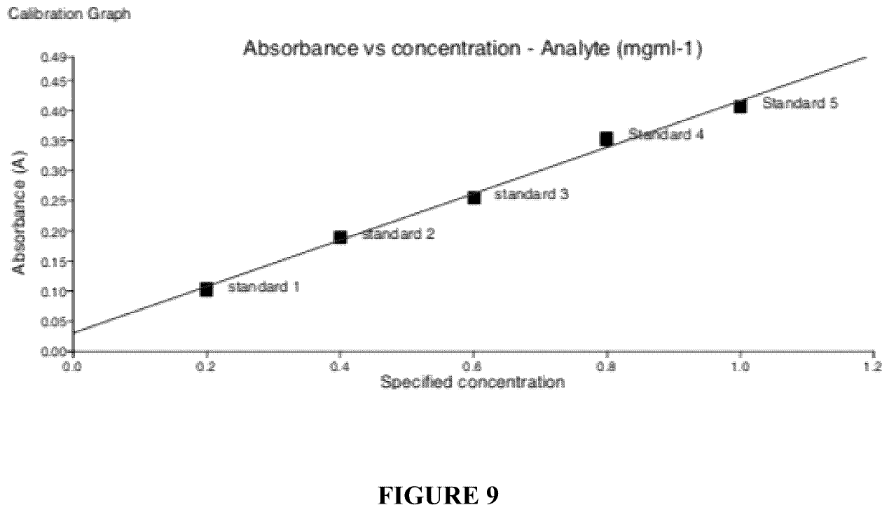

A standard curve was constructed using a glucosamine standard since the base molecule of the polyactive carbohydrate is glucosamine (see FIG. 9). Table 12 provides the numerical data used to construct the calibration curve:

TABLE 12
Glucosamine Calibration Curve

| | | Concentration | | |
|---|---|---|---|---|
| | Concentration | (based on | | |
| | (as prepared, | absorbance, | | Absorbance |
| Sample | mg/mL) | mg/mL) | Residual | (AU) |
| 1 | 0.2 | 0.1878 | 0.0122 | 0.1034 |
| 2 | 0.4 | 0.4146 | −0.0146 | 0.1907 |
| 3 | 0.6 | 0.5842 | 0.0158 | 0.2560 |
| 4 | 0.8 | 0.8369 | −0.0369 | 0.3533 |
| 5 | 1.0 | 0.9766 | 0.0234 | 0.4071 |

The culture extract prepared as described above had a concentration of 0.7195 mg/mL of polyactive carbohydrate. This was diluted to 0.3688 mg/mL for use in biofoam and cup production experiments.

### Example 13: Effect of Biofoam Waste on Plant Growth

The effect of waste from biofoam cup production on plants was assessed as follows. Corn or bean seeds were placed in soil without (control) or with granular waste from cup production. Growth of germinating seeds and leaf/plant color were evaluated over a 2-month period.

Average plant size and color for the treatment and control groups after two months can be seen in Table 13:

TABLE 13
Growth and Coloration of Plants Exposed to
Biofoam Production Waste after Two Months

| | Plant | Bean | Corn |
|---|---|---|---|
| Control | Height | 13.5 cm | 33 cm |
| | Color | Light Green | Light Green |
| Treatment | Height | 18 cm | 45 cm |
| | Color | Intense Green | Intense Green |

Intensity of color, as qualitatively assessed in Table 13, is reflective of an increase in chlorophyll content. Both bean and corn plants treated with granules of biofoam waste exhibited increased growth and chlorophyll production as compared to untreated controls.

### Example 14: Preparation of Adhesives from Polyactive Carbohydrate Extracts and Anti-Microbial/UV-Protective Extracts

The following procedures were used to prepare adhesives from extracts of the devices disclosed herein. These adhesives are 90% natural and 10% synthetic and the polyactive carbohydrate and anti-microbial/UV-protective extracts were prepared as described in Example 8.

At room temperature, 19.5 mL of each respective extract were mixed together with 1 mL of polysorbate 80 for 3 minutes. 40 mL of castor oil were added and the mixture was stirred for an additional 10 minutes.

Separately, and also at room temperature, 5 mL of each respective extract were mixed together with 0.5 g of bentonite and vortexed for 5 minutes. This mixture was added to the above solution and stirred for 9 minutes.

20 mL of isocyanate (MDI; Geos Quimica S.A.S.) were added to the above mixture over the course of 9 minutes at room temperature; this represented a ratio of approximately 4:1 polyol:isocyanate.

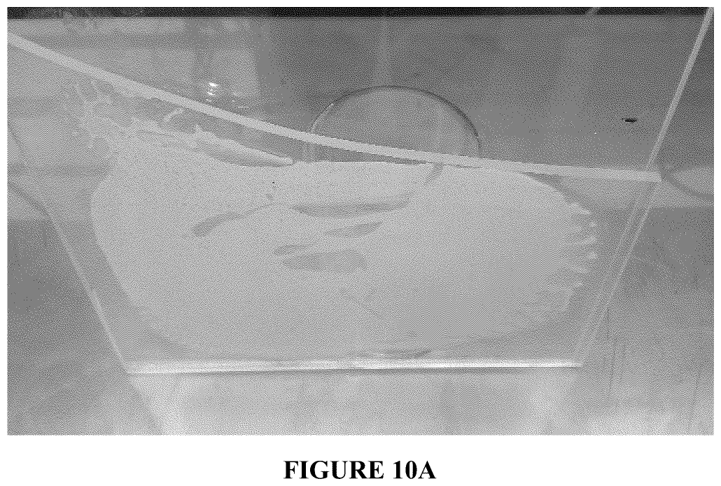

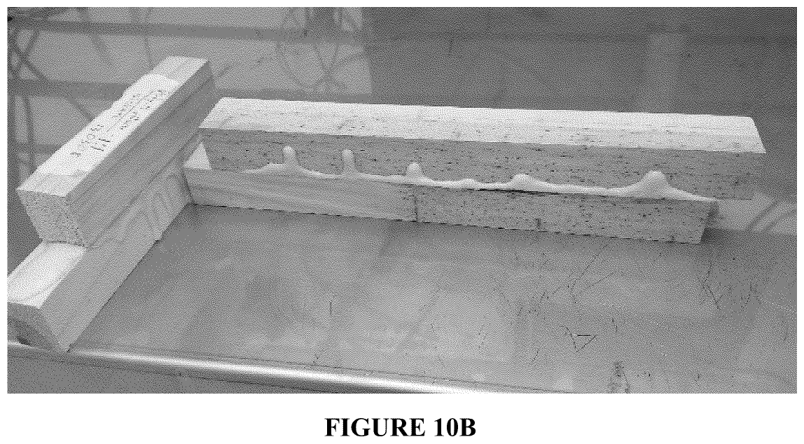

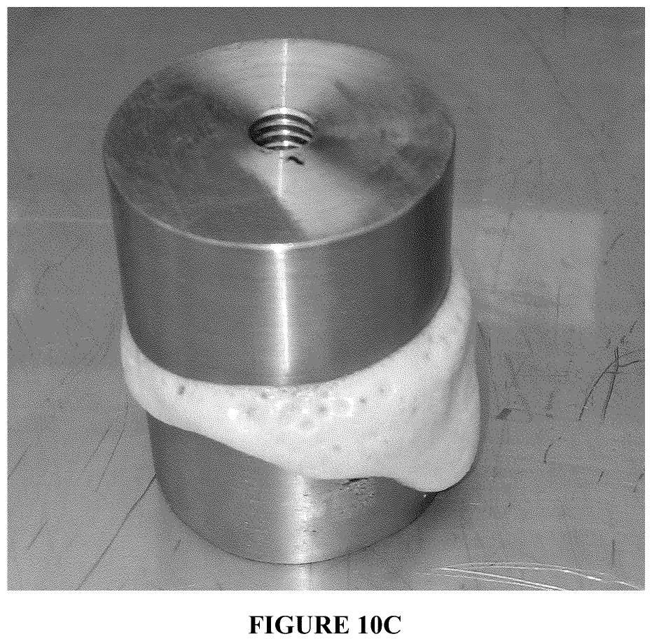

The mixture was then applied to the surfaces to be glued together. The adhesive has a drying time of two hours and was able to adhere plastic, wood, and metal (see FIGS. 10A-10C).

Because the adhesives are prepared from 90% natural materials, they are biodegradable.

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

1.-20. (canceled)

21. A method for increasing the UV resistance of a fruit or vegetable, the method comprising contacting the fruit or vegetable with an extract from a biological device, wherein the biological device comprises host cells transformed with a vector comprising a DNA construct comprising the following genetic components: (a) a gene that expresses zinc-related protein/oxidase, (b) a gene that expresses silicatein, (c) a gene that expresses silaffin, and (d) a gene that expresses alcohol dehydrogenase II.

22. The method of claim 21, wherein the DNA construct further comprises (e) a gene that expresses lipase.

23. The method of claim 21, wherein the vector is a plasmid selected from pWLNEO, PSV2CAT, pOG44, pXTI, pSG, pSVK3, pBSK, pBR322, pYES, pYES2, pBSKII, and pUC.

24. The method of claim 21, wherein the host cells comprise yeast or bacteria.

25. The method of claim 24, wherein the bacteria comprise Escherichia coli.

26. The method of claim 24, wherein the yeast comprise Saccharomyces cerevisiae.

27. The method of claim 21, wherein the gene that expresses zinc-related protein/oxidase has SEQ ID NO. 1 or at least 70% homology thereto.

28. The method of claim 21, wherein the gene that expresses silicatein has SEQ ID NO. 2 or at least 70% homology thereto.

29. The method of claim 21, wherein the gene that expresses silaffin has SEQ ID NO. 3 or at least 70% homology thereto.

30. The method of claim 21, wherein the gene that expresses alcohol dehydrogenase II has SEQ ID NO. 4 or at least 70% homology thereto.

31. The method of claim 22, where in the gene that expresses lipase has SEQ ID NO. 5 or at least 70% homology thereto.

32. The method of claim 21, wherein the DNA construct comprises from 5′ to 3′ the following genetic components in the following order: (1) the gene that expresses zinc-related protein/oxidase, (2) the gene that expresses silicatein, (3) the gene that expresses silaffin, and (4) the gene that expresses alcohol dehydrogenase II.

33. The method of claim 22, wherein the DNA construct comprises from 5′ to 3′ the following genetic components in the following order: (1) the gene that expresses lipase, (2) the gene that expresses zinc-related protein/oxidase, (3) the gene that expresses silicatein, (4) the gene that expresses silaffin, and (5) the gene that expresses alcohol dehydrogenase II.

34. The method of claim 21, wherein the extract is produced by exposing a culture of the biological device to UV radiation.

35. The method of claim 21, wherein the method is performed once.

36. The method of claim 21, wherein the method is performed weekly.

37. The method of claim 21, wherein an entire fruit or vegetable is contacted with the composition, or wherein an upper portion of a fruit or vegetable is contacted with the composition.

38. The method of claim 21, wherein performing the method increases at least one nutrient or flavor-enhancing component in a fruit or vegetable relative to an otherwise identical fruit or vegetable on which the method is not performed.

39. The method of claim 38, wherein the fruit comprises a watermelon or tomato and wherein the nutrient comprises lycopene.

40. The method of claim 21, wherein performing the method protects the fruit or vegetable against water loss.
